(19) United States
(12) Patent Application Publication
(10) Pub. No.: US 2024/0000959 A1
(43) Pub. Date: Jan. 4, 2024

(54) CANCER IMMUNOTHERAPIES TO PROMOTE HYPERACUTE REJECTION

(71) Applicant: CORNELL UNIVERSITY, Ithaca, NY (US)

(72) Inventors: Neil H. BANDER, Quogue, NY (US); Wilhem LECONET, Utrecht (NL); Ming GUO, Tenafly, NJ (US); He LIU, New York, NY (US); Ivo LORENZ, Brooklyn, NY (US); Abdul G. KHAN, Cedar Park, TX (US)

(21) Appl. No.: 18/039,369

(22) PCT Filed: Nov. 30, 2021
(86) PCT No.: PCT/US2021/061180

§ 371 Date: May 30, 2023

Related U.S. Application Data
(60) Provisional application No. 63/119,359, filed on Nov. 30, 2020.

(51) Int. Cl. A61K 47/66; C07K 16/28; C07K 16/30; A61P 35/00; C12N 9/10

(52) U.S. Cl. CPC A61K 47/67; C07K 16/2803; C07K 16/3069; A61P 35/00; C12N 9/1051; C12Y 204/01069; C12Y 204/01087; C07K 2317/55; C07K 2317/622; A61K 2039/505

(57) ABSTRACT

The present application relates to a bi-functional therapeutic for treating cancer that includes a targeting component which targets a tumor-associated antigen and an enzyme which, when delivered to a tumor by said targeting component, converts the tumor phenotype to that of an incompatible allograft or xenograft. The enzyme is coupled to the targeting component. Also disclosed is a method for treating cancer comprising administering the bi-functional therapeutic.

This application claims the priority benefit of U.S. Provisional Patent Application Ser. No. 63/119,359, filed Nov. 30, 2020, which is hereby incorporated by reference in its entirety.

### FIELD

The present disclosure relates to cancer immunotherapies to promote hyper-acute rejection.

### BACKGROUND

Combination therapy is a common, accepted treatment approach for virtually all types of cancers and has been the standard therapeutic approach for several decades. The basis for the adoption of combination therapy was the early chemotherapy experience where it was determined that the high mutational rate of cancers allowed rapid development of resistant strains of tumor cells when only a single agent was employed. The goal of combination therapies is to increase efficacy and minimize the development of tumor resistance or escape. This is generally achieved by employing 2 or more anti-cancer agents each of which has a different mechanism of action, making the development of resistant tumor cells more difficult and less likely. The additive or synergistic effects of combining two or more agents can be the difference between successful and unsuccessful treatment of the patient.

Many combination treatment regimens are well known in the oncology field. As an example, MOPP (an acronym for mechlorethamine, vincristine, procarbazine, prednisone) is a curative treatment regimen for Hodgkins' Disease. Several different combination regimens (which all include cisplatin, vinblastine, and bleomycin) are accepted in the treatment of testicular cancer, which is curable in up to 98% of diagnosed cases. In all, more than 300 different combination regimens have been used.

The main drawback to combination therapy is often that it also results in an increase in toxicity. For example, most forms of nonsurgical cancer therapy, such as external irradiation and chemotherapy, are limited in their efficacy because of toxic side effects to normal tissues and cells as well as the limited specificity of these treatment modalities for cancer cells. This limitation is also of importance when anti-cancer antibodies are used for targeting toxic agents, such as isotopes, drugs, and toxins, to cancer sites, because, as systemic agents, they also circulate to sensitive cellular compartments such as the bone marrow. In acute radiation injury, there is destruction of lymphoid and hematopoietic compartments as a major factor in the development of septicemia and subsequent death. Thus, methods of reducing the toxic effects of cancer therapy while maintaining or even increasing efficacy are in high demand.

In an alternative to combination therapy, recent advances in immunotherapy clearly establish that the immune system can be engaged to respond to cancer and that these responses can be quite effective and durable. The substantial experience with immune checkpoint inhibition suggests its greatest benefit lies in its application to cancers that harbor relatively high mutational burdens. But even in such cases only a minority of patients respond. Some cancers like prostate cancer lack immune cells in the tumor microenvironment. This absence of immune cells, sometimes referred to as a ‘cold’ microenvironment or an immunological ‘desert’ severely limits the ability to activate the immune system. Chimeric antigen receptor T (CAR-T) cells and bi-specific T cell engagers (BiTE) utilize antibody targeting of a tumor-associated antigen to direct the T-cell lytic machinery to lyse cancer cells. But thus far, CAR-T and BiTE anti-tumor activity has been limited to hematogenous cancers, not the far more common solid tumors. Clearly, there remains a need for additional methods to treat a variety of cancers.

The present disclosure is directed to overcoming these and other deficiencies in the art.

### SUMMARY

One aspect of the present disclosure relates to a bi-functional therapeutic for treating cancer that includes a targeting component which targets a tumor-associated antigen and an enzyme which, when delivered to a tumor by said targeting component, enzymatically converts the tumor phenotype to that of an incompatible allograft or xenograft. The enzyme is coupled to the targeting component.

Another aspect of the present disclosure relates to a method of treating cancer. This method involves selecting a subject having cancer; providing a bi-functional therapeutic according to the present disclosure; and administering, to the selected subject, the bi-functional therapeutic under conditions effective to treat the cancer.

Another aspect of the present disclosure relates to a bi-functional therapeutic for treating cancer that includes a targeting component which targets the prostate-specific membrane antigen (PSMA)/Folate hydrolase 1 (FOLH1) receptor and a glycosyltransferase which, when delivered to a tumor by said targeting component, enzymatically converts the tumor phenotype to that of an incompatible allograft or xenograft, said glycosyltransferase being coupled to said targeting component.

Another aspect of the present disclosure relates to a bi-functional therapeutic for treating cancer that includes a targeting component which targets a human epidermal growth factor receptor (HER) family member and a glycosyltransferase which, when delivered to a tumor by said targeting component, enzymatically converts the tumor phenotype to that of an incompatible allograft or xenograft, said glycosyltransferase being coupled to said targeting component.

Another aspect of the present disclosure relates to a bi-functional therapeutic for treating cancer that includes a targeting component which targets CD19 and a glycosyltransferase which, when delivered to a tumor by said targeting component, enzymatically converts the tumor phenotype to that of an incompatible allograft or xenograft, said glycosyltransferase being coupled to said targeting component.

A novel immuno-therapeutic approach is presented in which a tumor-targeted glycosyltransferase alters the glyco-phenotype of the tumor and/or it's blood vessels by adding a non-self histo-blood group antigen (HBGA) or alpha-gal glycotope. This effectively converts tumor to a HBGA-incompatible allograft or a xenograft. An exemplary embodiment of this multifunctional agent can target PSMA/FOLH1 to convert tumor neo-vasculature to a mismatched HBGA or xenograft thereby initiating hyper-acute rejection. A half-century of transplant experience documents that a HBGA-incompatible allograft or alpha-gal expressing xenograft stimulates a robust immune rejection process.

As described herein, to generate xeno- or alloantigen expression by tumor, xenogeneic or allogeneic glycosyltransferases, e.g., alpha gal Transferase (alpha galT) or allogeneic glycosyltransferase A and/or B enzyme, all normally resident in the Golgi, is delivered to the tumor cell surface—in effect a molecular-scale heterotopic allo/xenograft. Alternatively, the alpha galT, A and/or B enzymes can be targeted to antigens specific to tumor neo-vascular endothelial targets such as folate hydrolase 1 (FOLH1) (also known as prostate-specific membrane antigen (PSMA)), or vascular endothelial growth factor receptor-2 (VEGFR-2), or other targets known to those in the art. In addition to the targeting of the glycosyltransferase (alpha galT, glycosyltransferase A and/or B enzymes), the respective sugar-nucleotide donor (UDP-gal or UDP-NAcGal) is supplied. In the presence of the glycosyltransferse at the tumor, the sugar (gal or NAcGal) is added to the existing glycoproteins and glycolipids, including products secreted by the targeted cells, to generate the allo- or xeno-antigens thereby triggering a vigorous immune response. The converted allo/xeno proteins secreted into the microenvironment bind abundant natural antibodies triggering complement activation, an immune response, antibody-dependent cytotoxicity (ADCC) and serve to convert a “cold” microenvironment to a “hot” one.

Glycosyltransferase A and B enzymes differ by only 4 of their 353 amino acid residues (Hakomori, “Antigen Structure and Genetic Basis of Histo-Blood Groups A, B and O: Their Changes Associated With Human Cancer,” Biochimica et Biophysica Acta 1473:247-266 (1999); Seto et al., “Sequential Interchange of Four Amino Acids From Blood Group B to Blood Group A Glycosyltransferase Boosts Catalytic Activity and Progressively Modifies Substrate Recognition in Human Recombinant Enzymes,” J. Biol. Chem. 272:14133-14138 (1997), which are hereby incorporated by reference in their entirety) making them unlikely to be immunogenic. Studies of patient sera have confirmed that these enzymes are, as predicted, not immunogenic. Indeed, while their HBGA carbohydrate products are highly immunogenic, the transferase A and B enzymes have never been reported to be immunogenic. Tumor targeted delivery of a non-immunogenic transferase A or B enzyme thereby provides a means to alter the tumor or neo-vasculature immuno-phenotype into one that expresses a highly immunogenic non-self HBGA thereby assuming the phenotype of an incompatible allograft and prompting a robust rejection response by the host.

As described herein, for proof of concept, the approach was validated with the human-derived GTA or GTB. Alternatively, one could utilize the xenogeneic alpha-gal transferase (alpha 1,3 Galactosyltransferase; alpha-galT) enzyme that is mutated/non-functional in humans and responsible for causing the rejection of xenografted organs from other mammals. Use of the alpha-galT enzyme might require humanization or de-immunization of the alpha-galT, and there are methods known in the art to accomplish this including, but not limited to, using sequences of homologous regions of other glycosyltransferases that are not immunogenic to humans. Such humanization or de-immunization methods have been widely and successfully used to humanize or de-immunize foreign-derived antibodies prior to use as therapeutics in humans. However, studies of patient sera have shown that these enzymes are not immunogenic.

The present disclosure presents a novel immuno-therapeutic approach in which a tumor-targeted glycosyltransferase alters the histo-blood group antigen expression of the tumor and/or its blood supply. This effectively converts tumor to a HBGA-incompatible allograft. This multifunctional agent can be used to target PSMA/FOLH1 to convert tumor neo-vasculature to a mismatched HBGA thereby initiating hyper-acute rejection.

As described herein, a complementary, orthogonal immunotherapeutic approach was modeled on the robust immune response to a xeno- or allograft and the understanding of the rejection process that has developed over the past half-century. To achieve this, the most extreme form of host vs graft response: hyper-acute rejection (HAR), was chosen as a model.

HAR occurs as a result of ancestral mutations in either of 2 highly related genes: alpha 1,3 Galactosyltransferase (alpha 1,3 GalT) in the case of xenografts (Collins, et al., “Cardiac Xenografts Between Primate Species Provide Evidence for the Importance of the Alpha-Galactosyl Determinant in Hyperacute Rejection,” J. Immunol. 154:5500-5510 (1995), which is hereby incorporated by reference in its entirety) and the well-known histo-blood group antigen (HBGA) locus in the case of allografts (Milland et al., “ABO Blood Group and Related Antigens, Natural Antibodies and Transplantation,” Tissue Antigens 68:459-466 (2006), which is hereby incorporated by reference in its entirety). These two highly related genes are found on the same chromosome (9q34), bear 45% homology and are believed to have derived from the same ancestral gene (Yamamoto et al., “Molecular Genetic Basis of the Histo-Blood Group ABO System,” Nature 345:229-233 (1990); Yamamoto et al., “Sugar-Nucleotide Donor Specificity of Histo-Blood Group A and B Transferases is Based on Amino Acid Substitutions,” J. Biol. Chem. 265:19257-19262 (1990); Yamamoto et al., “Genomic Organization of Human Histo-Blood Group ABO Genes,” Glycobiology 5:51-58 (1995), which are hereby incorporated by reference in their entirety). These alleles code for glycosyltransferases that post-translationally add a terminal sugar moiety to the carbohydrate (CHO) chain present on nascent proteins and lipids destined for cell membrane expression or secretion. Due to mutation, the alpha GalT enzyme was inactivated in humans and old world monkeys, but not other mammals, about 28 million years ago (Macher et al., “The Gal Alpha1,3Gal Beta1,4GlcNAc-R (Alpha-Gal) Epitope: a Carbohydrate of Unique Evolution and Clinical Relevance,” Biochim. Biophys. 1780:75-88 (2008), which is hereby incorporated by reference in its entirety). As a result, xenografted organs and tissues derived from non-primate mammals express the alpha gal epitope that is foreign to humans. In the case of the HBGA locus, a small number of mutations have led to the alleles known classically as A, B and O. The B allele encodes Glycosyltransferase B (GTB) that, like its alpha 1,3 GalT homolog, adds a terminal Gal to the CHO chain, the sole difference being that transferase B adds the Gal only if a 1,2 fucose is present on the adjacent Gal. Transferase A differs functionally from Transferase B only in that it adds a terminal Gal that is N-acetylated (NAcGal). The O gene product is inactive due to a frameshift mutation (FIG. 1).

The alpha-Gal, HBGA A and HBGA B epitopes generated by these 3 active enzymes are expressed widely in nature including bacteria that inhabit the human gut (Springer et al., “Blood Group Isoantibody Stimulation in Man by Feeding Blood Group-Active Bacteria,” J. Clin. Invest. 48:1280-1291 (1969), which is hereby incorporated by reference in its entirety). As a result, humans lacking the aGalT and the A and/or B alleles are being continuously immunized by these bacterially derived epitopes. This leads to very high levels of natural antibodies (Abs) to these non-self epitopes that constitute greater than 1% of plasma immunoglobulin (Ig) (Galili et al., “One Percent of Human Circulating B Lymphocytes are Capable of Producing the Natural Anti-Gal Antibody,” Blood 82:2485-2493 (1993); Galili et al., “A Unique Natural Human IgG Antibody With Anti-Alpha-Galactosyl Specificity,” J. Exp. Med. 160:1519-1531 (1984), which are hereby incorporated by reference in their entirety). Given the diversity of the Ab repertoire estimated to be in the billions of different specificities, this represents an enormous proportion of endogenous Ig activity. These Abs are composed of IgMs, and IgGs that activate the complement cascade which, in turn, can initiate vascular thrombosis (Subramaniam et al., “Distinct Contributions of Complement Factors to Platelet Activation and Fibrin Formation in Venous Thrombus Development,” Blood 129(16):2291-2302 (2017); Foley et al., “Cross Talk Pathways Between Coagulation and Inflammation,” Circ. Res. 118:1392-1408 (2016); and Conway E M, “Reincarnation of Ancient Links Between Coagulation and Complement,” J. Thromb. Haemost. 13(Suppl. 1):S121-S32 (2015), which are hereby incorporated by reference in their entirety). Other immunoglobulin classes such as IgA and IgE can also be directed to these glycol-epitopes. In effect, evolutionary mutations in these two genes create an immunological state poised at a tipping point, primed and ready to respond rapidly, aggressively and destructively to the appearance of any of these non-self epitopes. The immunological effects of these mutations have precluded successful xeno-transplants in humans and explain why HBGA matching is the single most important match in solid organ transplantation since its critical importance was first recognized by Starzl, Experience In Renal Transplantation. (WB Saunders Company, Philadelphia, PA, chapter 6 (1964), which is hereby incorporated by reference in its entirety, in the early days of renal allografts in the 1960's. Since that time, the disastrous effects of a HBGA mismatch in solid organ transplants is seen only in those very rare instances when iatrogenic errors occur (Altman, Doctors Discuss Transplant Mistake. New York Times, Feb. 22, 2003, which is hereby incorporated by reference in its entirety). This background context led to the goal to induce expression of one of these non-self epitopes by the host's cancer cells and/or the vascular endothelial cells that supply the tumor.

### BRIEF DESCRIPTION OF THE DRAWINGS

FIG. 1 shows the strict acceptor substrate specificity of glycosyltransferases. The B (or A)-transferase will only add its respective sugar to glycosylation sites that express the H-antigen. Fortuitously, absence of this requisite H-antigen in many normal tissues prevents off-target conversion to HBGA A or B. Alternatively, in the event that one desires to intentionally target a normal or cancerous cell type that naturally lacks the H-antigen, this can be accomplished in a manner analogous to that described for adding A or B by also targeting the alpha1-2 fucosyltransferase and providing GDP-fucose as the fucose donor. Addition of the fucose/H-antigen can be done simultaneously with the targeted A or B transferase or the additions can be done in a step-wise manner (e.g., first the fucose, then the A or B addition).

FIGS. 2A-2B shows that chimeric Ab-GTB protein maintains immunoreactivity and enzymatic activity. FIG. 2A is a graph showing that the J591-GTB chimeric protein maintains comparable binding immunoreactivity to PSMA relative to the parental J591 antibody measured by ELISA. FIG. 2B is a bar graph showing that the chimeric protein also retains enzymatic activity demonstrated by its ability to catalyze the transfer of ¹⁴C-galactose from UDP-¹⁴C-galactose, the nucleotide donor, to 2′-fucosyl-lactose (2-FL). This incorporation occurs to a high level only when the J591-GTB fusion protein and its acceptor substrate, 2-FL, are present. Similar results were obtained with anti-4D5 (her2)-GTB.

FIG. 3 is a graph showing that GTB activity can be modulated by C-terminal extension. J591-GTB activity (% of control) is shown as a function of increasing length of C-terminal amino acid extension and measured by incorporation of ¹⁴C-gal from UDP-¹⁴C-gal to 2′-fucosyl-lactose (2-FL).

FIG. 4 are images showing that J591-GTB specifically converts antigen-positive tumor cells. Tissue sections from a CWR22Rv1 xenograft (heterogeneously PSMA+/HBGA O), were incubated with J591-GTB+UDP-gal and immunohistochemically stained for HBGA B expression (left panel). Negative control sections including secondary anti-murine Ig-peroxidase but lacking mouse anti-HBGA B (middle panel) or J591-GTB (right panel), respectively, did not stain.

FIG. 5 are images showing the effect of J591-GTB on LNCaP and PC3 cells. LNCaP cells (PSMA⁺/HBGA 0; left panel) converted by J591-GTB to HBGA B; PC3 cells (PSMA⁻/HBGA 0; right panel) do not undergo conversion by J591-GTB.

FIGS. 6A-6D are images showing that PC3 cells transfected with PSMA then treated with J591-GTB. FIG. 6A shows phase contrast images. FIG. 6B shows cells expressing PSMA. FIG. 6C shows HBGA B antigen expression. FIG. 6D is a merge of FIGS. 6B and 6C. Only those cells expressing PSMA were converted to HBGA B expression. PSMA-neg cells, primarily at left center and top center, remain HBGA B-neg.

FIG. 7 are images showing LNCaP cells spiked into a suspension of Type O RBCs and incubated with J591 (Top row); J591-GTB (middle row), or J591-GTB-54aa extension (bottom row). The left column shows phase contrast image. The middle column shows DAPI nuclear stain. The right column shows murine anti-HBGA B+goat anti-mouse IgM-alexa488. While the PSMA-pos LNCaP cells are converted to HBGA B-pos by J591-GTB, with or without the C-terminal extension, bound to their plasma membrane, the PSMA-neg RBCs are not converted.

FIGS. 8A-8D are images showing complement-mediated lysis in vitro. LNCaP cells were incubated with either native mAb J591 or mAb J591-GTB fusion protein. All wells also got UDP-gal. Subsequently, serum from a type A patient was added as a source of natural anti-B Ab and complement. The combination of J591-GTB plus type A serum (FIG. 8A) led to complete LNCaP lysis. The J591-GTB fusion protein did not induce lysis in the absence of type A serum (FIG. 8B). Without the fusion protein, no lysis was detected regardless of the presence (FIG. 8C) or absence of type A serum (FIG. 8D).

FIG. 9 are images showing complement-mediated cytotoxicity of several cell lines. Complement-mediated cytotoxicity of several cell lines as observed by trypan blue exclusion is shown. The upper panel was treated with J591-GTB+UDP-gal+human type O serum. Cells in the lower panel were treated with the same O serum but without J591-GTB+UDP-gal. The proportion of dead cells is reported under each photograph as determined by FACS (FIG. 10). For the FACS, type O serum, without J591-GTB+UDP-gal, served as a negative control, whereas 0.1% triton exposure provided a complete lysis control.

FIGS. 10A-10H are images showing the in vivo conversion of prostate and breast cancers to HBGA B. FIGS. 10A-10D show serial sections through LNCaP xenograft in SCID mouse 24 hours after administration of PBS+UDP-gal (FIG. 10A), b) J591+UDP-gal (FIG. 10B), and J591-GTB+UDP-gal (FIG. 10C). FIGS. 10A-10C are immunohistochemically stained for HBGA B (all 10×). FIG. 10D shows a serial section from same specimen stained for PSMA. See also FIGS. 12A-12E for higher power and additional xenograft lines. FIGS. 10E-10H show MD-MB361 breast cancer (HER2+) xenograft after treatment with PBS+UDP-gal (FIG. 10E), 4D5+UDP-gal (FIG. 10F), 4D5-GTB+UDP-gal (FIGS. 10G and 10H). Sections are immunohistochemically stained for HBGA B expression. Discrete plasma membrane staining is apparent. In FIGS. 10C and 10H, adjacent connective tissue does not get converted, demonstrating that the specificity of the immuno-phenotypic conversion is restricted to targeted tumor.

FIGS. 11A-11B are a bar graph (FIG. 11A) and histograms (FIG. 111B) showing lysis of B-converted cell lines by type O serum as determined by propidium iodide uptake measured by FACS and trypan blue exclusion (see FIG. 9). O serum in the absence of B-conversion does not cause lysis. After treating PSMA-pos cells with J591-GTB+UDP-gal, the type O serum completely lysed all of the PSMA-pos/B-converted cell lines; PC3, which is HBGA O-pos/PSMA-neg, did not convert to HBGA B and was not lysed.

FIGS. 12A-12E are images showing in vivo conversion of LNCaP, C4-2 and CWR22Rv1 xenografts by J591-GTB. FIGS. 12A-12B show LNCaP xenograft treated in vivo with: J591 [without GTB] (FIG. 12A) or J591-GTB (FIG. 12B), both with UDP-gal, immunohistochemically stained with mouse anti-HBGA B; high power. FIG. 12C shows C4-2 prostate cancer treated in vivo with J591-GTB plus UDP-gal, immunohistochemically stained with mouse anti-HBGA B; high power. FIGS. 12D-12E show CWR22Rv1 prostate cancer, heterogeneously and weakly PSMA-pos, treated in vivo with J591-GTB plus UDP-gal. Adjacent connective tissue is not converted to HBGA B.

FIGS. 13A-13B are a graph (FIG. 13A) and in vivo images of mice (FIG. 13B) showing in vivo conversion of HBGA and treatment. Mice were implanted I.P. with 10×10⁶ C4-2-luc cells suspended in Matrigel. Several days later, bioluminescence was measured and 10 mice with confirmed viable tumor were randomly assigned to one of 2 treatment arms. All tumor-bearing mice received a single dose of J591-GTB+UDP-gal+human type O serum; in half of the mice, the serum was heat-inactivated prior to injection. In those mice treated with active type O serum, the mean photon flux decreased progressively over the ensuing 13 days whereas those with inactivated serum experienced mean tumor progression. At the end of the experiment on day 13, the difference in bioluminescence between groups was significant (p<0.0032). A duplicate experiment yielded consistent results.

FIGS. 14A-14B are in vivo images and a graph showing the results of experiment #2 in which C4-2-luc cells were implanted IP followed later by a single treatment with J591-GTB+UDP-gal+human type O serum (upper rows) (FIG. 14A). FIG. 14A shows images of mice receiving active type O serum or type O serum which had been previously heat-inactivated. Mice receiving heat-inactivated serum demonstrated tumor progression (see plot of photon flux; FIG. 14B) whereas those getting active serum experienced tumor regression; experiment 1 results are shown in FIGS. 13A-13B.

FIG. 15 is a FACS histogram showing CD19, CD20, and CD38 expression in MM1-S cells. Flow cytometry analysis showed the MM1-S multiple myeloma cell line is CD38 positive, CD19 positive, and CD20 negative.

FIGS. 16A-16B are FACS histograms showing ABO expression of MM1-S cells. FIG. 16A shows the MM1-S multiple myeloma cells line is A/B negative. FIG. 16B shows MM1-S multiple myeloma cells line is O positive.

FIG. 17 is a FACS histogram showing that CD19⁺/O⁺ MM1-S myeloma cells can be converted to B⁺ by GTB+UDP-gal. The GTB can be targeted to myeloma cells using anti-CD19, anti-CD38, or anti-BCMA.

FIG. 18 are images demonstrating that the use of ACUPA, a small molecule ligand that binds to PSMA, conjugated to GTB (ACUPA-GTB), to direct conversion of LNCaP from HBGA O to HBGA B. This demonstrates that, in addition to antibody (or antibody derivatives), a small molecule ligand or peptide that binds the target antigen on the tumor cell or neo-vascular endothelium can also be used for purposes of targeting the enzyme. The left panel shows ACUPA-PEG-1500-GTB treated cells. The right panel shows cells treated with GTB only.

FIG. 19 are images showing the specificity of the conversion from HBGA O to HBGA B. SK-BR5 breast cancer cells (PSMA⁻/O⁺) were co-cultured with LNCaP prostate cancer cells (PSMA⁺/O⁺). The two cell types can be distinguished by morphology: SK-BR5 are round whereas LNCaP cells are elliptical/spindle. In addition, the LNCaP cells are marked with green fluorescent protein (GFP). Incubation with J591-GTB and UDP-gal converts only the PSMA⁺ LNCaP cells but not the neighboring cells that lack the PSMA target. Panels show DAPI (left panel), GFP (middle panel), and Anti-B (Cy5) (right panel) imaging.

FIG. 20 are images showing the specificity of the conversion from HBGA O to HBGA B. As shown, only PSMA⁺ cells are converted to B⁺ by J591-GTB/UDP-gal.

FIGS. 21A-21B are FACS histograms showing the specificity of the conversion from HBGA O to HBGA B. The specificity of conversion was quantified using FACS by comparing the concentration of J591 (anti-PSMA)-GTB required to convert LNCaP (PSMA⁺) to HBGA B (FIG. 21A) relative to SK-BR5 (PSMA-neg) cells (FIG. 21B). Both cell lines are O⁺. FACS histograms are shown. No B conversion of SK-BR5 occurs even at concentrations of J591-GTB up to 100 μg/mL. By comparison, concentrations as low as 0.012 μg/mL induce the conversion of the PSMA-positive LNCaP cells.

FIG. 22 is a table and graph showing the specificity of the conversion from HBGA O to HBGA B. A table (left panel) and histogram of MFI from FIGS. 21A-21B is shown (right panel). Specificity index exceeds 8,000:1.

FIG. 23 is a table and graph showing that both cell surface and secreted glycoproteins are glycosylated by the method of the present disclosure. A graph of cell counts (top panel) and table (bottom panel) are shown.

FIGS. 24A-24B are plots showing testing for anti-α1,3GalT antibodies in serum samples. FIG. 24B is an expanded view of FIG. 24A showing the lower optical densities.

FIG. 25 is an SDS-PAGE gel showing expression and purification of recombinant proteins.

FIGS. 26A-26B are graphs showing binding of scfv-CD19-αGal to CD19⁻ MM1.S cells (FIG. 26A) and CD19⁺ Raji cells) (FIG. 26B).

FIG. 27 is a graph showing a galactose transfer assay on a mixture of CD19⁺ and CD19⁻ cells.

FIG. 28 are histograms showing a galactose transfer assay on CD19⁺ cells.

FIG. 29 are scatter plots showing binding and αGal transfer testing of scfv-αGT to human B-cells.

FIG. 30 is a dot plot showing a serum mediated lysis assay on CD19⁺ cells.

FIGS. 31A-31B are graphs showing a lysis assay on αGal transferred B-cells. FIG. 31A is a graph showing % lysis. FIG. 31B is a graph showing IgG levels (MFI) and IgM levels.

FIGS. 32A-32C show an in vitro checkerboard assay of scfv-CD19-αGT and UDP-Gal. FIG. 32A measures binding, FIG. 32B measures alpha gal expression, and FIG. 32C measures lysis by human PBMCs.

FIG. 33 is a bar graph showing the % remaining B-cells at baseline and at 1 hour, 4 hours, 1 day, 7 days, 14 days, 30 days, and 60 days following the administration of anti-CD19 scFv-alpha Gal Transferase fusion protein and UDP-gal. B-cell counts were determined by examining CD20⁺/CD3⁻ fluorescence. CD20 was used to avoid confounding the B-cell count by presence of anti-CD19 scFv.

### DETAILED DESCRIPTION

The present disclosure teaches a bi-functional therapeutic for treating cancer that includes a targeting component which targets a tumor-associated antigen and an enzyme which, when delivered to a tumor by said targeting component, enzymatically converts the tumor phenotype to that of an incompatible allograft or xenograft. The enzyme is coupled to the targeting component.

The targeting component can be antibody derived (intact, monovalent single chain, Fab′2, Fab, scFv or other) or a peptide. The targeting and enzyme moieties can be linked via generation of a fusion gene/protein or via biochemical conjugation.

The present disclosure also pertains to a method of treating cancer. The method involves selecting a subject having cancer and providing a bi-functional therapeutic according to the present disclosure. The bi-functional therapeutic is administered, to the selected subject, under conditions effective to treat the cancer.

As used herein, the term “treat” refers to the application or administration of the bi-functional therapeutic of the present disclosure to a subject, e.g., a patient. The treatment can be to cure, heal, alleviate, relieve, alter, remedy, ameliorate, palliate, improve or affect the cancer, the symptoms of the cancer or the predisposition toward the cancer.

As used herein, the term “subject” is intended to include human and non-human animals. Non-human animals include all vertebrates, e.g., mammals and non-mammals, such as non-human primates, sheep, dog, cow, chickens, amphibians, reptiles, etc.

As used herein, the term “cancer” includes all types of cancerous growths or oncogenic processes, metastatic tissues or malignantly transformed cells, tissues, or organs, irrespective of histopathologic type or stage of invasiveness.

As used herein, an “incompatible allograft” refers to a tissue or tumor that induces hyper-acute, acute and/or chronic immune rejection. Hyper-acute rejection appears in minutes to a few hours following organ transplantation, or, as described herein, after conversion of a tumor or tissue upon delivery of a bifunctional therapeutic. This rapid rejection is characterized by vessel thrombosis leading to graft/tumor necrosis. Hyperacute rejection is caused by the presence of anti-donor antibodies existing in the recipient before transplantation/conversion.

As used herein, the “targeting component” is a component that is able to bind to or otherwise associate with a tumor-associated antigen. Such tumor associated antigens include, but are not limited to the following as well as their peptide fragments: FOLH1/PSMA, VEGFR, CD19, CD20, CD25, CD30, CD33, CD38, CD52, B cell Maturation Antigen (BCMA), CD79, Somatostatin receptor, 5T4, gp100, Carcinoembryonic antigen (CEA), mammoglobin A, melan A/MART-1, MAGE, NY-ESO-1, PSA, tyrosinase, HER-2/neu, HER-3, EGFR, hTERT, mesothelin, Nectin-4, TROP-2, Tissue Factor, MUC-1, CA-125, and peptide fragments thereof, protein MZ2-E, polymorphic epithelial mucin, folate-binding protein, cancer testis proteins MAGE-1 or MAGE-3 or NY-ESO-1, Human chorionic gonadotropin (HCG), Alpha fetoprotein (AFP), Pancreatic oncofetal antigen, CA-15-3, 19-9, 549, 195, Squamous cell carcinoma antigen (SCCA), Ovarian cancer antigen (OCA), Pancreas cancer associated antigen (PaA), mutant K-ras proteins, mutant p53, nonmutant p53, truncated epidermal growth factor receptor (EGFR), chimeric protein p210BCR-ABL, telomerase, survivin, WT1 protein, LMP2 protein, HPV E6 E7 protein, Idiotype protein, and PAP protein. The preceding list exemplifies tumor-associated antigens; additional tumor-associated antigens are known to those in the art.

The antigen may be an antigen or epitope present, for example, on a tumor cell located within the lungs, breast, esophagus, intestine, stomach, rectum, renal-urinary system, prostate, bladder, brain, thyroid, liver, pancreas, spleen, skin, connective tissue, heart, blood system, or vascular system. The target antigen may be an antigen or epitope present on a cell membrane, secreted protein, or on a non-membrane bound protein. Examples of secreted proteins include, but are not limited to hormones, enzymes, toxins and antimicrobial peptides.

The targeting component may become localized or converge at a particular targeted site, for instance, a tumor, a disease site, a tissue, an organ, a type of cell, an infectious bacteria or virus, etc.

For example, contemplated targeting components include a peptide, polypeptide, protein, glycoprotein, aptamer, carbohydrate, or lipid. A targeting component may be a naturally occurring or synthetic ligand for a cell surface receptor, e.g., a growth factor, hormone, LDL, transferrin, etc. A targeting component can be an antibody, which term is intended to include antibody fragments and derivatives, characteristic portions of antibodies, single chain targeting moieties which can be identified, for example, using procedures such as phage display. Targeting components may also be a targeting peptide, targeting peptidomimetic, or a small molecule, whether naturally-occurring or artificially created (e.g., via chemical synthesis).

In one embodiment, the targeting component is selected from the group consisting of an antibody or antigen-binding fragment thereof, a protein, a peptide, and aptamer, and a small molecule.

Antibodies against tumor-associated antigens are known. For example, antibodies and antibody fragments which specifically bind markers produced by or associated with tumors have been disclosed, inter alia, in U.S. Pat. No. 3,927,193 to Hansen, and U.S. Pat. Nos. 4,331,647, 4,348,376, 4,361,544, 4,468,457, 4,444,744, 4,818,709 and 4,624,846 to Goldenberg, which are hereby incorporated by reference in their entirety. In particular, antibodies against a tumor-associated antigen, e.g., a gastrointestinal, lung, breast, prostate, ovarian, testicular, brain or lymphatic or hematogenous tumor, a sarcoma or a melanoma, are advantageously used. Antibodies to tumor-associated antigens are well known to those in the art.

The antibodies of the present disclosure may exist in a variety of forms including, for example, polyclonal antibodies, monoclonal antibodies, intracellular antibodies (“intrabodies”), antibody fragments (e.g. Fv, Fab and F(ab)2), half-antibodies, hybrid derivatives, as well as single chain antibodies (scFv), chimeric antibodies and de-immunized or humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Bird et al, “Single-Chain Antigen-Binding Proteins,” Science 242:423-426 (1988), each of which is hereby incorporated by reference in its entirety).

Antibodies of the present disclosure may also be generated using recombinant DNA technology, such as, for example, an antibody or fragment thereof expressed by a bacteriophage. Alternatively, the synthetic antibody is generated by the synthesis of a DNA molecule encoding and expressing the antibody of the present disclosure or the synthesis of an amino acid sequence specifying the antibody, where the DNA or amino acid sequence has been obtained using synthetic DNA or amino acid sequence technology which is available and well known in the art.

Methods for monoclonal antibody production may be carried out using the techniques described herein or are well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest either in vivo or in vitro.

Alternatively monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified using recombinant DNA technology to generate alternative antibodies or derivatives. For example, the constant domains of the light and heavy chains of a mouse monoclonal antibody can be substituted by those regions derived from a human antibody to generate a chimeric antibody. Alternatively, the constant domains of the light and/or heavy chains of a monoclonal antibody can be substituted by a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

The monoclonal antibody of the present disclosure can be a humanized antibody. Humanized antibodies are antibodies that contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

In addition to whole antibodies, the present disclosure encompasses antigen binding portions of such antibodies. Such binding portions include the monovalent Fab fragments, Fv fragments (e.g., single-chain antibody, scFv), and single variable V_{H} and V_{L} domains, and F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES:PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antibody mimics are also suitable for use in accordance with the present disclosure. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

In certain embodiments, the targeting component targets the prostate-specific membrane antigen (PSMA) receptor.

As used herein, “PSMA” or “prostate-specific membrane antigen” protein refers to mammalian PSMA, preferably human PSMA protein. PSMA is sometimes referred to as folate hydrolase 1 (FOLH1) as PSMA is encoded by the FOLH1 gene. The long transcript of PSMA encodes a protein product of about 100-120 kDa molecular weight characterized as a type II transmembrane receptor having sequence homology with the transferrin receptor and having NAALADase activity (Carter et al., “Prostate-Specific Membrane Antigen is a Hydrolase With Substrate and Pharmacologic Characteristics of a Neuropeptidase,” Proc. Natl. Acad. Sci. USA 93:749-753 (1996); Israeli et al., “Molecular Cloning of a Complementary DNA Encoding a Prostate-Specific Membrane Antigen,” Cancer Research 53:227-230 (1993), which are hereby incorporated by reference in their entirety).

Monoclonal anti-PSMA antibodies can be used as the targeting component in the bi-functional therapeutic of the present disclosure. Preferably, the monoclonal antibodies bind to the extracellular domain of PSMA (i.e., an epitope of PSMA located outside of a cell such as at about amino acids 44-750 of human PSMA, of which the amino acid residues correspond to the human PSMA sequence disclosed in U.S. Pat. No. 5,538,866, which is hereby incorporated by reference in its entirety)). Examples of murine monoclonal antibodies to human PSMA include, but are not limited to, E99, J415, J533 and J591, which are produced by hybridoma cell lines having an ATCC Accession Number HB-12101, HB-12109, HB-12127, and HB-12126, respectively, all of which are disclosed in U.S. Pat. Nos. 6,107,090 and 6,136,311, which are hereby incorporated by reference in their entirety. Most preferably, the murine monoclonal antibody is J591, produced by HB-12126, or de-immunized J591 antibody described in U.S. Pat. Nos. 7,045,605 and 7,514,078 to Bander et al., which are hereby incorporated by reference in their entirety.

In some embodiments the targeting component targets an HER receptor family member. An exemplary targeting component of an HER receptor family member is monoclonal antibody 4D5.

In certain embodiments, the targeting component is a peptide that binds to the tumor-associated antigen. Exemplary peptides include, without limitation, glutamate-urea-lysine derivatives such as 2-(3-99S)-5-amino-1-carboxypentyl)ureido) Pentanedioic acid (ACUPA) that binds FOLH1/PSMA, somatostatin derivatives that bind SSTR2, and Arg-Gly-Asp (RGD) peptide that binds alpha-v/beta-3 integrin.

The peptides used in conjunction with the present disclosure can be obtained by known isolation and purification protocols from natural sources, can be synthesized by standard solid or solution phase peptide synthesis methods according to the known peptide sequence of the peptide, or can be obtained from commercially available preparations or peptide libraries. Included herein are peptides that exhibit the biological binding properties of the native peptide and retain the specific binding characteristics of the native peptide. Derivatives and analogs of the peptide, as used herein, include modifications in the composition, identity, and derivitization of the individual amino acids of the peptide provided that the peptide retains the specific binding properties of the native peptide. Examples of such modifications would include modification of any of the amino acids to include the D-stereoisomer, substitution in the aromatic side chain of an aromatic amino acid, derivitization of the amino or carboxyl groups in the side chains of an amino acid containing such a group in a side chain, substitutions in the amino or carboxy terminus of the peptide, linkage of the peptide to a second peptide or biologically active moiety, and cyclization of the peptide (G. Van Binst and D. Tourwe, “Backbone Modifications in Somatostatin Analogues: Relation Between Conformation and Activity,” Peptide Research 5:8-13 (1992), which is hereby incorporated by reference in its entirety).

As used herein, “small molecules” are typically organic, peptide or non-peptide molecules, having a molecular weight less than 10,000 Da, preferably less than 5,000 Da, more preferably less than 1,000 Da, and most preferably less than 500 Da. This class of modulators includes chemically synthesized molecules, for instance, compounds from combinatorial chemical libraries.

In certain embodiments, the targeting component is an aptamer. Aptamers are small single-stranded DNA or RNA oligonucleotides that specifically bind to their target molecules (e.g., a tumor-associated antigen) with high affinity and specificity. Aptamers are created using an in vitro selection process termed systematic evolution of ligands by exponential enrichment (SELEX), which is described in Ellington et al., “In Vitro Selection of RNA Molecules That Bind Specific Ligands,” Nature 346:818-822 (1990) and Jayasena, “Aptamers: An Emerging Class of Molecules That Rival Antibodies in Diagnostics,” Clin. Chem. 45:1628-1650 (1999), which are hereby incorporated by reference in their entirety. Several aptamers capable of targeting tumor-associated antigens including, without limitation, MUC1, HER2, HER3, EpCAM, NF-kB, PSMA, CD44, PD-1, CD137, CD134, PDGF, VEGF, and NCL have been developed (Jayasena, “Aptamers: An Emerging Class of Molecules That Rival Antibodies in Diagnostics,” Clin. Chem. 45:1628-1650 (1999), which is hereby incorporated by reference in its entirety).

As used herein, the term “enzyme” encompasses any enzyme, protein or peptide which, when delivered to a tumor or tissue by a targeting component, catalyzes the conversion of the tumor or tissue to an incompatible allograft.

In one embodiment, the enzyme is an enzyme involved in post-translational modification and is selected from the group consisting of a transferase and a glycosyltransferase.

A transferase is any one of a class of enzymes that enact the transfer of specific functional groups (e.g. a methyl or glycosyl group) from one molecule (called the donor) to another (called the acceptor).

An exemplary group of transferases includes, without limitation, glycosyltransferases. Glycosyltransferases catalyze the addition of activated sugars (donor NDP-sugars), in a step-wise fashion, to a protein, glycoprotein, lipid or glycolipid or to the non-reducing end of a growing oligosaccharide (Lairson et al., “Glycosyltransferases: Structures, Functions, and Mechanisms,” Annu. Rev. Biochem. 77:521-55 (2008), which is hereby incorporated by reference in its entirety). Glycosyltransferases are well known in the art.

Mammals utilize 9 sugar nucleotide donors for glycosyltransferases: UDP-glucose, UDP-galactose, UDP-GlcNAc, UDP-GalNAc, UDP-xylose, UDP-glucuronic acid, GDP-mannose, GDP-fucose, and CMP-sialic acid.

For enzymatic saccharide syntheses that involve glycosyltransferase reactions, glycosyltransferase can be cloned, or isolated from any source. Many cloned glycosyltransferases are known, as are their polynucleotide sequences (see, e.g., “The WWW Guide To Cloned Glycosyltransferases,” Taniguchi et al., 2002, Handbook of Glycosyltransferases and Related Genes, Springer, Tokyo, which is hereby incorporated by reference in its entirety). Glycosyltransferase amino acid sequences and nucleotide sequences encoding glycosyltransferases from which the amino acid sequences can be deduced are also well known in the art.

Glycosyltransferases that can be employed in the methods of the present disclosure include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligoglycosyltransferases. Suitable glycosyltransferases include those obtained from eukaryotes, as well as from prokaryotes.

Glycosyltransferases are critical for the genesis of the ABO blood group antigen system. As described supra, the ABO blood system is the primary antigen system important in blood transfusion and solid organ transplantation. This histo-blood group antigen (HBGA) system is controlled by the activity of GTA and/or GTB glycosyltransferases that attach sugar residues (N-acetylgalactosamine or galactose) to a common substrate (the H antigen). The enzyme has several phenotypic variants which either alter the carbohydrate attached (N-acetylgalactosamine (A) vs galactose (B)) or cause loss of function of the enzyme so the H antigen is not modified (O). A variant of A, A2, has a reduced level of N-acetylgalactosamine activity and NAc-gal addition. These variants are discriminated currently by serology and by lectin binding (defining A1 vs A2). Serology can either detect the modification of the H antigen or can detect the presence of naturally-occurring antibodies directed to A and/or B (e.g., a person with the B pattern of glycosylation will have antibodies directed to A).

In humans the glycosyltransferase locus, referred to herein as the ABO locus or the ABO glycosyltransferase locus, is located on chromosome 9 and contains seven exons that span more than 18 kb of genomic DNA. Exon 7 is the largest and contains most of the coding sequence. The ABO locus has three main allelic forms: A, B, and O. The A “allele” (also referred to as A1 or A2) encodes a glycosyltransferase that enzymatically adds N-acetylgalactosamine to the D-galactose end of the H antigen, producing the so-called A antigen. The B allele encodes a glycosyltransferase that enzymatically adds D-galactose to the D-galactose end of the H antigen, thus creating the so-called B antigen. The O allele encodes a nonfunctional form of glycosyltransferase, resulting in an unmodified H antigen, creating the so-called O antigen phenotype.

On the genomic level, the ABO glycosyltransferase gene has many alleles (˜300). These naturally occurring allelic variants are described in Yip, “Sequence Variation at the Human ABO Locus,” Ann. Hum. Genet. 66:1-27 (2002); Hakomori “Antigen Structure and Genetic Basis of Histo-Blood Group A, B, and O: Their Changes Associated with Human Cancer,” Biochimica et Biophysica Acta 1473:247-266 (1999); Seto et al., “Sequential Interchange of Four Amino Acids from Blood Group B to Blood Group A Glycosyltransferase Boosts Catalytic Activity and Progressively Modifies Substrate Recognition in Human Recombinant Enzymes,” J. Biol. Chem. 272:14133-14138 (1997), which are hereby incorporated by reference in their entirety, and their use in the bi-functional therapeutic of the present disclosure are contemplated. The sequence encoding the catalytic site of the enzyme lies in exon 7 of the gene; key amino acid residues 176, 235, 266, and 268 control the specificity of this active site. Furthermore, a common nucleotide deletion in exon 6 creates a stop codon that abolishes synthesis of full-length glycosyltransferase, leading to the O or null phenotype.

Thus, in some embodiments, the glycosyltransferase is selected from the group consisting of glycosyltransferase A (alpha 1-3-N-acetylgalactosaminlytransferase), glycosyltransferase B (alpha 1-3-galactosyltransferase), alpha-gal-transferase, and glycosyltransferase A (Gly268Ala). Allelic variants, as described supra, are also contemplated.

In some embodiments, a glycosyltransferase used in the method of the present disclosure is a fucosyltransferase. Fucosyltransferases are known to those of skill in the art. Exemplary fucosyltransferases include enzymes which transfer L-fucose from GDP-fucose to a hydroxy position of an acceptor sugar. Fucosyltransferases that transfer non-nucleotide sugars to an acceptor are also of use in the present disclosure.

In some embodiments, the glycosyltransferase is a humanized or de-immunized glycosyltransferase. Methods of humanizing and/or de-immunizing proteins are known in the art.

Accordingly, one embodiment of the present disclosure relates to the alteration of the blood group antigen expression on a tumor and/or the blood supply of the tumor by a tumor-targeted glycosyltransferase. As described supra, this effectively converts the tumor phenotype to that of an incompatible allograft or xenograft thereby initiating hyper-acute rejection.

The bi-functional therapeutic described herein may be formed such that the targeting component is a protein or peptide linked to the enzyme via a peptide bond.

In certain embodiments, the protein or peptide targeting component linked to the enzyme via a peptide bond may be referred to as a chimeric or fusion protein. As used herein, the term “chimeric protein” or “fusion protein” encompasses a polypeptide having a single continuous polypeptide chain, i.e., a series of contiguous amino acids linked by peptide bonds or a series of polypeptide chains covalently or non-covalently linked to one another (i.e., a polypeptide complex) that includes at least a portion of a full-length sequence of first polypeptide sequence and at least a portion of a full-length sequence of a second polypeptide sequence, where the first and second polypeptides are different polypeptides. A chimeric polypeptide also encompasses polypeptides that include two or more non-contiguous portions derived from the same polypeptide. A chimeric polypeptide or protein also encompasses polypeptides having at least one substitution, wherein the chimeric polypeptide includes a first polypeptide sequence in which a portion of the first polypeptide sequence has been substituted by a portion of a second polypeptide sequence. The series of polypeptide chains can be covalently linked using a suitable biochemical linker or a disulfide bond.

Coupling of the targeting component and the enzyme can also be prepared using chemical linkage (Brennan et al., “Preparation of Bispecific Antibodies by Chemical Recombination of Monoclonal Immunoglobulin G1 Fragments,” Science 229:81-3 (1985), which is hereby incorporated by reference in its entirety) or chemical coupling (Shalaby et al., “Development of Humanized Bispecific Antibodies Reactive With Cytotoxic Lymphocytes and Tumor Cells Overexpressing the HER2 Protooncogene,” J. Exp. Med. 175:217-225 (1992), which is hereby incorporated by reference in its entirety).

In other embodiments, the targeting component and the enzyme may be linked via non-covalent bonds including, without limitation, hydrogen bonds, ionic bonds, Van der Waals forces, and hydrophobic interactions.

Thus, fusion or linkage between a targeting component (e.g. antibody) and an enzyme may be achieved by conventional covalent or ionic bonds, protein fusions via genetic engineering, or heterobifunctional crosslinkers, e.g., carbodiimide, glutaraldehyde, and the like. Conventional inert linker sequences (e.g. peptide linkers) which simply provide for a desired amount of space between the targeting component and the enzyme may also be used. The design of such linkers is well known to those of skill in the art and is described for example in U.S. Pat. Nos. 8,580,922; 5,525,491; and 6,165,476, which are hereby incorporated by reference in their entirety. A variety of coupling or cross-linking agents can be used for covalent conjugation of proteins. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al., “Production of Target-Specific Effector Cells Using Hetero-Cross-Linked Aggregates Containing Anti-Target Cell and Anti-Fc Gamma Receptor Antibodies,” J. Exp. Med. 160(6):1686-701 (1984); Liu et al., “Heteroantibody Duplexes Target Cells for Lysis by Cytotoxic T Lymphocytes,” Proc. Natl. Acad. Sci. USA 82(24):8648-52 (1985), which are hereby incorporated by reference in their entirety). Other methods include those described in Paulus, Behring Ins Mitt No 78, 1 18-132 (1985); Brennan et al., “Preparation of Bispecific Antibodies by Chemical Recombination of Monoclonal Immunoglobulin G1 Fragments,” Science 229:81-83 (1985); Glennie et al., “Preparation and Performance of Bispecific F(ab′ gamma)2 Antibody Containing Thioether-Linked Fab′ Gamma Fragments,” J. Immunol. 139:2367-2375 (1987), which are hereby incorporated by reference in their entirety).

A number of other linkers can be used to couple the targeting component to the enzyme. For example, a disulfide linkage can be used, as described in Saito et al., Adv. Drug Delivery Reviews 55:199-215 (2003), which is hereby incorporated by reference in its entirety. Linkers that are sensitive to the lower pH found in endosomes or in the tumor environment can also be used, including hydrazones, ketals and/or aconitic acids. A hybrid linker can also be used, e.g., a linker with two or more potential cleavage sites, e.g., a disulfide and a hydrazone. Peptidase-sensitive linkers can also be used, e.g., tumor-specific peptidases, for example, linkers sensitive to cleavage by PSA. PEG linkers can also be used (Wiiest et al., Oncogene 21:4257-4265 (2002), which is hereby incorporated by reference in its entirety). Exemplary linkers include hydrazone and disulfide hybrid linkers (see Hamann et al., Bioconjugate Chem. 13:47-58 (2002); Hamann et al., Bioconjug Chem. 13(1):40-6 (2002), which are hereby incorporated by reference in their entirety); SPP (Immunogen); and a variety of linkers available from Pierce Biotechnology, Inc. In some embodiments, the linker is SSP (a disulfide linker, available from Immunogen), and the ratio of linker to antibody can be varied from, e.g., 7:1 to 4:1. Various spacer and linker sequences are known in the art and are described in Chen et al., “Fusion Protein Linkers: Property, Design and Functionality,” Adv. Drug Deliv. Rev. 65(10):1357-69 (2013), which is hereby incorporated by reference in its entirety.

The term ‘peptide linker’ or spacer refers to a short peptide fragment that connects or couples the targeting component and the enzyme moieties of the polypeptide of the bi-functional therapeutic. The linker is preferably made up of amino acids linked together by peptide bonds. For example, the peptide linker can comprise small amino acid residues or hydrophilic amino acid residues (e.g. glycine, serine, threonine, proline, aspartic acid, asparagine, etc). For example, the peptide linkers are peptides with an amino acid sequence with a length of at least 5 amino acids, or with a length of about 5 to about 100 amino acids, or with a length of about 10 to 50 amino acids, or a length of about 10 to 15 amino acids.

In one example, the linker is made up of a majority of amino acids that are sterically unhindered such as glycine and alanine. Thus in a further example, the linkers are polyglycines, polyalanines or polyserines.

One skilled in the art would appreciate that many commonly used peptide linkers may be used in embodiments of the present disclosure. In certain embodiments, the short peptide linkers may comprise repeat units to increase the linker length. For example, a double, triple or quadruple repeated linker. In one example, the linker comprises a formula (Gly-Gly-Gly-Gly-Ser)n (SEQ ID NO:1) or comprising the formula (Ser-Gly-Gly-Gly-Gly)n Ser (SEQ ID NO:2) wherein n is a number from 3 to 6. In some embodiments, the linker is a (G₄S)₃ linker (SEQ ID NO: 67).

Non-peptide linkers or spacers are also possible. For example, alkyl linkers such as —NH—(CH₂)s-C(O)—, wherein s=2-20 could be used. These alkyl linkers may be further substituted by any non-sterically hindering group such as lower alkyl (e.g. C1-C6), lower acyl, halogen (e.g. Cl, Br), CN, NH₂, phenyl. An exemplary non-peptide linker is a PEG linker or spacer having a molecular weight of 100 to 5000 kD, preferably 1000 to 2000 kD, and more preferably 1500 kD.

A bifunctional therapeutic according to the present disclosure may include an N-terminus coupled to a C-terminus. N-terminus and C-terminus are used herein to refer to the N-terminal region or portion and the C-terminal region or portion, respectively, of the bifunctional therapeutic protein of the present disclosure. In some embodiments of the present disclosure, the C-terminal portion and the N-terminal portion of the bifunctional therapeutic of the present disclosure are contiguously joined. In alternative embodiments, the C-terminal portion and the N-terminal portion of the bifunctional therapeutic of the present disclosure are coupled by an intervening spacer. In one embodiment, the spacer may be a polypeptide sequence of 1, 2, 3, 4, 5, 6, 7, 8, 9, 10, or more amino acid residues. In some embodiments, the C-terminal portion and/or the N-terminal portion of the bifunctional therapeutic of the present disclosure may include additional portion(s) coupled to the C-terminal residue and/or the N-terminal residue of the chimeric protein of the present disclosure, respectively. In some embodiments, the additional portion(s) may be a polypeptide sequence of 1, 2, 3, 4, 5, 6, 7, 8, 9, 10, or more amino acid residues. In some embodiments, the N-terminal portion and/or the C-terminal portion having such additional portion(s) will maintain the activity of the corresponding naturally occurring N-terminal portion of a targeting component and/or C-terminal portion of an enzyme, respectively. In some embodiments, the N-terminal portion and/or the C-terminal portion having such additional portion(s) will have enhanced and/or prolonged activity compared to the corresponding naturally occurring N-terminal portion of a targeting component and/or C-terminal portion of an enzyme, respectively. In other embodiments, the C-terminal portion and/or the N-terminal portion of the bifunctional therapeutic of the present disclosure do not include any additional portion(s) coupled to the C-terminal residue and/or the N-terminal residue of the chimeric protein of the present disclosure, respectively.

In one embodiment, the N-terminal region comprises the targeting component. In certain embodiments, the targeting component is an antibody or antigen-binding portion thereof including, without limitation, monomeric single chain antibodies, Fab fragments, Fab′2, scFv, and other antibody fragment derivatives such as minibodies, diabodies, and triabodies. The antibodies or antigen-binding fragments may maintain or delete the FcRn-binding domain.

In one embodiment, the N-terminal region comprises human J591 heavy chain and has an amino acid sequence of SEQ ID NO:3 (GenBank Accession No. CCA78124.1, which is hereby incorporated by reference in its entirety), or a portion thereof, as follows:

EVQLQQSGPE LVKPGTSVRI SCKTSGYTFT EYTIHWVKQS
HGKSLEWIGN INPNNGGTTY NQKFEDKATL TVDKSSSTAY
MELRSLTSED SAVYYCAAGW NFDYWGQGTT LTVSS

In another embodiment, the N-terminal region comprises human J591 light chain and has an amino acid sequence of SEQ ID NO:4 (GenBank Accession No. CCA78125.1, which is hereby incorporated by reference in its entirety), or a portion thereof, as follows:

DIVMTQSHKF MSTSVGDRVS IICKASQDVG TAVDWYQQKP
GQSPKLLIYW ASTRHTGVPD RFTGSGSGTD FTLAITNVQS
EDLADYFCQQ YNSYPLTFGA GTKLEIKR

In another embodiment, the N-terminal region comprises human 4D5 heavy chain and has an amino acid sequence of SEQ ID NO:5, or a portion thereof, as follows:

EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKENW
YVDGVEVHNA KTKPREEQYN STYRVVSVLT VLHQDWLNGK
EYKCKVSNKA LPAPIEKTIS KAKGQPREPQ VYTLPPSREE
MTKNQVSLTC LVKGFYPSDI AVEWESNGQP ENNYKTTPPV
LDSDGSFFLY SKLTVDKSRW QQGNVESCSV MHEALHNHYT
QKSLSLSPGK

In another embodiment, the N-terminal region comprises human 4D5 light chain and has an amino acid sequence of SEQ ID NO:6, or a portion thereof, as follows:

DIQMTQSPSS LSASVGDRVT ITCRASQDVN TAVAWYQQKP
GKAPKLLIYS ASFLYSGVPS RESGSRSGTD FTLTISSLQP
EDFATYYCQQ HYTTPPTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSEN RGEC

In accordance with the above, in some embodiments the C-terminal region comprises the enzyme.

In one embodiment, the C-terminal region comprises the catalytic domain of glycosyltransferase B (GTB) and has an amino acid sequence of SEQ ID NO:7 (GenBank Accession No. AM423112.1, which is hereby incorporated by reference in its entirety), or a portion thereof, as follows:

MAEVLRTLAG KPKCHALRPM ILFLIMLVLV LFGYGVLSPR
SLMPGSLERG FCMAVREPDH LQRVSLPRMV YPQPKVLTPC
RKDVLVVTPW LAPIVWEGTF NIDILNEQFR LQNTTIGLTV
FAIKKYVAFL KLFLETAEKH FMVGHRVHYY VFTDQPAAVP
RVTLGTGRQL SVLEVGAYKR WQDVSMRRME MISDFCERRF
LSEVDYLVCV DVDMEFRDHV GVEILTPLFG TLHPSFYGSS
REAFTYERRP QSQAYIPKDE GDFYYMGAFF GGSVQEVQRL
TRACHQAMMV DQANGIEAVW HDESHLNKYL LRHKPTKVLS
PEYLWDQQLL GWPAVLRKLR FTAVPKNHQA VRNP

In another embodiment, the C-terminal region comprises the “cis A,B” sequence, which generates a hybrid sequence of GTB and GTA and has an amino acid sequence of SEQ ID NO:8 (GenBank Accession No. ABL75287.1, which is hereby incorporated by reference in its entirety), or a portion thereof, as follows:

YVAFLKLFLE TAEKHFMVGH RVHYYVFTDQ PAAVPRVTLG
TGRQLSVLEV GAYKRWQDVS MRRMEMISDF CERRFLSEVD
YLVCVDVDME FRDHVGVEIL TPLFGTLHPS FYGSSREAFT
YERRPQSQAY IPKDEGDFYY MGGFFGGSVQ EVQRLTRACH
QAMMVDQANG IEAVWHDESH LNKYLLRHKP TKVLSPEYLW
DQQLLGWPAV LRKLRFTAVP KNHQAVRNP

In another embodiment, the C-terminal region comprises the catalytic domain of glycosyltransferase A (GTA) and has an amino acid sequence of SEQ ID NO:9 (GenBank Accession No. AFB74122.1, which is hereby incorporated by reference in its entirety), or a portion thereof, as follows:

MAEVLRTLAG KPKCHALRPM ILFLIMLVLV LFGYGVLSPR
SLMPGSLERG FCMAVREPDH LQRVSLPRMV YPQPKVLTPC
RKDVLVVTPW LAPIVWEGTF NIDILNEQFR LQNTTIGLTV
FAIKKYVAFL KLFLETAEKH LMVGHRVHYY VFTDQPAAVP
RVTLGTGRQL SVLEVRAYKR WQDVSMRRME MISDFCERRF
LSEVDYLVCV DVDMEFRDHV GVEILTPLFG TLHPGFYGSS
REAFTYERRP QSQAYIPKDE GDFYYLGGFF GGSVQEVQRL
TRACHQAMMV DQANGIEAVW HDESHLNKYL LRHKPTKVLS
PEYLWDQQLL GWPAVLRKLR FTAVPKNHQA VRNP

In certain embodiments, the tumor having the tumor-associated antigen expresses the H-antigen. As used herein, “the H-antigen” refers to an oligosaccharide chain having a terminal disaccharide fucose-galactose, where the fucose has an alpha-(1-2)-linkage. The H-antigen is produced by a fucosyltransferase and is the building block for the production of the A or B antigens within the ABO blood group system.

Accordingly, the present disclosure also pertains to a method of treating cancer. The method involves selecting a subject having cancer and providing a bi-functional therapeutic according to the present disclosure. The bi-functional therapeutic is administered to the selected subject, under conditions effective to treat the cancer.

Virtually any tumor expressing an H-antigen can be treated with the bifunctional therapeutic described herein, including, but not limited to prostate tumors, adrenocortical carcinoma tumors, anal tumors, appendix tumors, astrocytoma (childhood cerebellar or cerebral), basal-cell carcinoma, bile duct tumors, bladder tumors, bone tumors, osteosarcoma/malignant fibrous histiocytomas, brain stem gliomas, ependymomas, medulloblastomas, breast tumors, bronchial adenomas/carcinoids, Burkitt's lymphomas, carcinoid tumors, cervical tumors, childhood tumors, chondrosarcomas, colon tumors, cutaneous T-cell lymphomas, desmoplastic small round cell tumors, endometrial tumors, esophageal tumors, Ewing's sarcomas, retinoblastomas, gallbladder tumors, gastric (stomach) tumors, gastrointestinal stromal tumors, germ cell tumors, gestational trophoblastic tumors, head and neck tumors, heart tumors, hepatocellular (liver) tumors, Hodgkin lymphomas, hypopharyngeal tumors, islet cell carcinomas (endocrine pancreas), Kaposi sarcomas, kidney tumors, laryngeal tumors, lip and oral cavity tumors, non-small cell lung tumors, small cell lung tumors, lymphomas, melanomas, Merkel cell tumors, mesotheliomas, multiple endocrine neoplasia, multiple myelomas, nasopharyngeal tumors, neuroblastomas, oligodendrogliomas, oral tumors, oropharyngeal tumors, ovarian tumors, pancreatic tumors, pleuropulmonary, primary central nervous system lymphomas, retinoblastomas, rhabdomyosarcomas, salivary gland tumors, soft tissue sarcomas, uterine sarcomas, skin tumors (non-melanoma), small intestine tumors, squamous cell carcinomas, stomach tumors, testicular tumors, throat tumors, thymoma and thymic carcinomas, thyroid tumors, trophoblastic tumors, and urethral tumors.

Some cancers including, but not limited to, hematopoietic or lymphoid cancers, mesodermally derived cancers, sarcomas, neuroectodermal cancers, etc may not express the H antigen. This can be easily determined by flow cytometry or immunohistochemistry of a tumor sample using Ulex lectin binding to reveal the presence or absence of H. When H is absent, treatment using the current application can be accomplished in two ways: one may employ a targeted fucosyltransferase in order to add the H antigen prior to or simultaneous with a targeted glycosyltransferase as previously described. Alternatively, one may target the alpha galT enzyme which can add a terminal galactose and does not require the presence of the 1,2 fucose (H antigen).

In one embodiment, the targeting component of the bi-functional therapeutic targets the PSMA receptor on tumor vascular endothelium. PSMA expression has been reported in the tumor neo-vasculature of a variety of tumors but is absent in normal tissue vasculature. Exemplary tissue types that have PSMA-positive vascular endothelium include, without limitation, renal, lung, colon, gastric, breast, brain, pancreatic, hepatic, bladder, esophageal, adrenal, head and neck, melanoma, and brain tumors. Other embodiments include targeting PSMA expressed on the surface of prostate cancer cells, targeting HER2 on breast and other HER2-positive cancers, targeting CD19 on B-cell lineage cancers, and targeting CEA on colorectal cancers. Other applicable targets are described supra.

Some aspects of the present disclosure relate to a bi-functional therapeutic that includes a targeting component comprising the amino acid sequence of one, two, three, four, five, or six CDRs as provided in Tables 1 and 2 herein. In some embodiments, the targeting component comprises a modified amino acid sequence, where the modified amino acid sequence has at least 80% sequence identity to any one, two, three, four, five, or six of the CDR sequences provided in Tables 1 and 2.

TABLE 1
Heavy Chain CDR Sequences of Suitable Targeting Component Antibodies

| | HCDR1 | | HCDR2 | | HCDR3 | |
|---|---|---|---|---|---|---|
| mAb/Fab | | SEQ ID | | SEQ ID | | SEQ ID |
| clone name | Sequence | NO: | Sequence | NO: | Sequence | NO: |
| J591* | GYTFTEYTIH | 10 | NINPNNGGTTYNQKFED | 13 | GWNFDY | 16 |
| 4D5** | GFNIKDTYIH | 11 | RIYPTNGYTRYADSVKG | 14 | WGGDGFYAMDYW | 17 |
| obexelimab*** | SYVMH | 12 | YINPYNDGTKYNEKFQG | 15 | GTYYYGTRVFDY | 18 |
| (XmAb5871) | | | | | | |

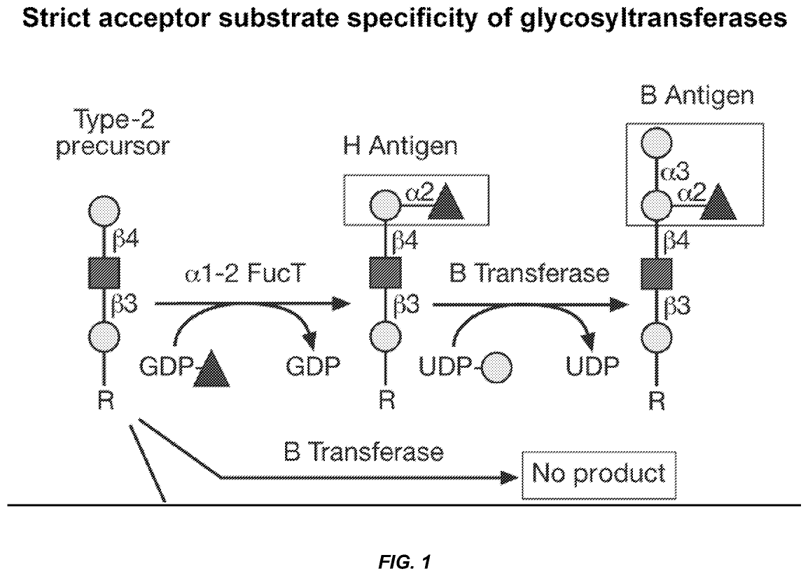

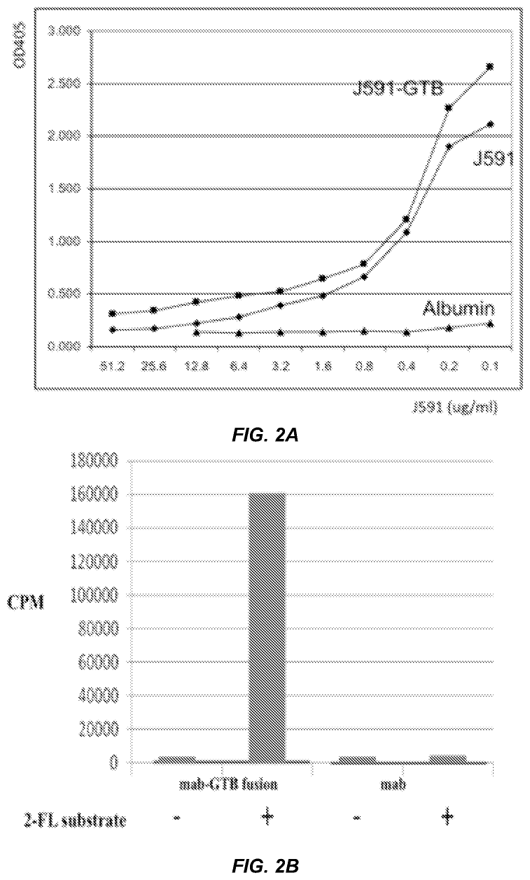

*See U.S. Pat. Appl. Publ. No. 2006/0088539, FIGS. 2A-2B, which is hereby incorporated by reference in its entirety;
**see U.S. Pat. No. 5,821,337, FIGS. 1A-1B, which is hereby incorporated by reference in its entirety;
***see EP2059536; PCT/US2007/075932, which is hereby incorporated by reference in its entirety.

TABLE 2
Light Chain CDR Sequences of Suitable Targeting Component Antibodies

| | LCDR1 | | LCDR2 | | LCDR3 | |
|---|---|---|---|---|---|---|
| mAb/Fab | | SEQ ID | Sequence | SEQ ID | | SEQ ID |
| clone name | Sequence | NO: | | NO: | Sequence | NO: |
| J591* | KASQDVGTAVD | 19 | WASTRHT | 22 | QQYNSYPLT | 25 |
| 4D5** | RASQDVNTAVAW | 20 | SASFLYS | 23 | QQHYTTPP | 26 |
| Obexelimab*** | RSSKSLQNVNGNTYLY | 21 | RMSNLNS | 24 | MQHLEYPIT | 27 |
| (XmAb5871) | | | | | | |

*See U.S. Pat. Appl. Publ. No. 2006/0088539, FIGS. 2A-2B, which is hereby incorporated by reference in its entirety;
***see U.S. Pat. No. 5,821,337, FIGS. 1A-1B, which is hereby incorporated by reference in its entirety;
***see EP2059536; PCT/US2007/075932, which is hereby incorporated by reference in its entirety.

In some embodiments, the heavy chain and/or the light chain variable regions of the antibody-based molecule described herein further comprises human or humanized immunoglobulin heavy chain and/or light chain framework regions, respectively.

In some embodiments of the present disclosure, the targeting component comprises one or two of the sequences provided in Table 3 herein. In some embodiments, the targeting component comprises a modified amino acid sequence, where the modified amino acid sequence has at least 80% sequence identity to any one or two of the sequences provided in Table 3.

TABLE 3
Antibody Variable Heavy (V_{H}) and Variable Light (V_{L}) Antibody Sequences

| mAb/Fab | | | SEQ ID |
|---|---|---|---|
| clone name | Region | Sequence^{†} | NO: |
| J591* | V_{H} | EVQLVQSGPEVKKPGATVKISCKTSGYTFTEYTIHWVKQAPGKG | 28 |
| | | LEWIGNINPNNGGTTYNQKFEDKATLTVDKSTDTAYMELSSLRS | |
| | | EDTAVYYCAAGWNFDYWGQGTLLTVSS | |
| J591* | V_{L} | DIQMTQSPSSLSTSVGDRVTLTCKASQDVGTAVDWYQQKPGP | 29 |
| | | SPKLLIYWASTRHTGIPSRFSGSGSGTDFTLTISSLQPEDFADYY | |
| | | CQQYNSYPLTFGPGTKVDIK | |
| Trastuzumab | V_{H} | EVQLVESGGGLVQPGGSLRLSCAASGFNIKDTYIHWVRQAPGK | 30 |
| (4D5)** | | GLEWVARIYPTNGYTRYADSVKGRFTISADTSKNTAYLQMNSLR | |
| | | AEDTAVYYCSRWGGDGFYAMDYWGQGTLVTVSS | |
| Trastuzumab | V_{L} | DIQMTQSPSSLSASVGDRVTITCRASQDVNTAVAWYQQKPGKA | 31 |
| (4D5)** | | PKLLIYSASFLYSGVPSRFSGSRSGTDFTLTISSLQPEDFATYYC | |
| | | QQHYTTPPTFGQGTKVEIKRT | |
| Obexelimab*** | V_{H} | EVQLVESGGGLVKPGGSLKLSCAASGYTFTSYVMHWVRQAPG | 32 |
| (XmAb5871) | | KGLEWIGYINPYNDGTKYNEKFQGRVTISSDKSISTAYMELSSLR | |
| | | SEDTAMYYCARGTYYYGTRVFDYWGQGTLVTVSS | |
| Obexelimab*** | V_{L} | DIVMTQSPATLSLSPGERATLSCRSSKSLQNVNGNTYLYWFQQ | 33 |
| (XmAb5871) | | KPGQSPQLLIYRMSNLNSGVPDRFSGSGSGTEFTLTISSLEPED | |
| | | FAVYYCMQHLEYPITFGAGTKLEIK | |

^{†}Complementarity-determining regions are shown in bold typeface.
*See U.S. Pat. Appl. Publ. No. 2006/0088539, FIGS. 2A-2B, which is hereby incorporated by reference in its entirety;
**see U.S. Pat. No. 5,821,337, FIGS. 1A-1B, which is hereby incorporated by reference in its entirety;
***see EP2059536; PCT/US2007/075932, which is hereby incorporated by reference in its entirety.

Suitable amino acid modifications to the heavy chain CDR sequences and/or the light chain CDR sequences of the targeting domain disclosed herein include, for example, conservative substitutions or functionally equivalent amino acid residue substitutions that result in variant CDR sequences having similar or enhanced binding characteristics to those of the CDR sequences disclosed herein as described above. Encompassed by the present disclosure are CDRs of Table 1 and 2 containing 1, 2, 3, 4, 5, or more amino acid substitutions (depending on the length of the CDR) that maintain or enhance binding of the antibody to its target (e.g., PSMA, CD14, HER2). The resulting modified CDRs are at least 70%, at least 75%, at least 80%, at least 85%, at least 90%, at least 95% similar in sequence to the CDRs of Tables 1 and 2. Suitable amino acid modifications to the heavy chain CDR sequences of Table 1 and/or the light chain CDR sequences of Tables 1 and 2 include, for example, conservative substitutions or functionally equivalent amino acid residue substitutions that result in variant CDR sequences having similar or enhanced binding characteristics to those of the CDR sequences of Table 1 and Table 2. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981, which is hereby incorporated by reference in its entirety). Non-conservative substitutions can also be made to the heavy chain CDR sequences of Table 1 and the light chain CDR sequences of Table 2. Non-conservative substitutions involve substituting one or more amino acid residues of the CDR with one or more amino acid residues from a different class of amino acids to improve or enhance the binding properties of CDR. The amino acid sequences of the heavy chain variable region CDRs of Table 1 and/or the light chain variable region CDRs of Table 2 may further comprise one or more internal neutral amino acid insertions or deletions that maintain or enhance target (e.g., PSMA, CD19, HER2) binding.

In some embodiments, the V_{H} chain of the targeting domain comprises any one of the V_{H} amino acid sequences provided in Table 3 above, or an amino acid sequence that is at least 60%, at least 65%, at least 70%, at least 75%, at least 80%, at least 85%, at least 90%, at least 95% identical to any one of the VH amino acid sequences listed in Table 3. For example, the targeting domain described herein may comprise: (i) a heavy chain variable region comprising an amino acid sequence that is at least 80% identical to SEQ ID NO: 30; (ii) a heavy chain variable region comprising an amino acid sequence that is at least 80% identical to SEQ ID NO: 32; or (iii) a heavy chain variable region comprising an amino acid sequence that is at least 80% identical to SEQ ID NO: 34.

In some embodiments, the V_{L} chain of the targeting domain comprises any one of the V_{L} amino acid sequences provided in Table 3 above, or an amino acid sequence that is at least 60%, at least 65%, at least 70%, at least 75%, at least 80%, at least 85%, at least 90%, at least 95% identical to any one of the V_{L} amino acid sequences listed in Table 3. For example, the targeting domain described herein may comprise: (i) a light chain variable region comprising an amino acid sequence that is at least 80% identical to SEQ ID NO: 29; (ii) a light chain variable region comprising an amino acid sequence that is at least 80% identical to SEQ ID NO: 31; or (iii) a light chain variable region comprising an amino acid sequence that is at least 80% identical to SEQ ID NO: 33.

In some embodiments, the targeting domain disclosed herein comprises: (i) a heavy chain variable region comprising an amino acid sequence that is at least 80% identical to SEQ ID NO: 30 and a light chain variable region comprising an amino acid sequence that is at least 80% identical to SEQ ID NO: 29; (ii) a heavy chain variable region comprising an amino acid sequence that is at least 80% identical to SEQ ID NO: 32 and a light chain variable region comprising an amino acid sequence that is at least 80% identical to SEQ ID NO: 31; or (iii) a heavy chain variable region comprising an amino acid sequence that is at least 80% identical to SEQ ID NO: 33 and a light chain variable region comprising an amino acid sequence that is at least 80% identical to SEQ ID NO: 32.

The targeting domains of the present disclosure may be described or specified in terms of their binding affinities. Thus, in some embodiments, the targeting domains of the present disclosure include those with a dissociation constant or K_{D} less than 1 μM, 500 nM, 250 nM, 200 nM, 100 nM, 50 nM, 40 nM, 30 nM, 25 nM, 20 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM.

Some aspects of the present disclosure relate to a bi-functional therapeutic for treating cancer that includes a targeting component which targets the prostate-specific membrane antigen (PSMA)/Folate hydrolase 1 (FOLH1) receptor and a glycosyltransferase which, when delivered to a tumor by said targeting component, enzymatically converts the tumor phenotype to that of an incompatible allograft or xenograft, said glycosyltransferase being coupled to said targeting component. This aspect of the present disclosure is useful in treating a subject with prostate cancer.

In some embodiments, the targeting component comprises a heavy chain variable region, where said heavy chain variable region includes: a complementarity-determining region 1 (CDR-H1) comprising an amino acid sequence of SEQ ID NO: 10, or a modified amino acid sequence of SEQ ID NO: 10, said modified sequence having at least 80% sequence identity to SEQ ID NO: 10; a complementarity-determining region 2 (CDR-H2) comprising an amino acid sequence of SEQ ID NO: 13, or a modified amino acid sequence of SEQ ID NO: 13, said modified sequence having at least 80% sequence identity to SEQ ID NO: 13; and a complementarity-determining region 3 (CDR-H3) comprising an amino acid sequence of SEQ ID NO: 16, or a modified amino acid sequence of SEQ ID NO: 16, said modified sequence having at least 80% sequence identity to SEQ ID NO: 16. The sequences of the heavy chain CDR sequences are provided in Table 1 above.

In some embodiments, the targeting component comprises a heavy chain variable region including an amino acid sequence that is at least 80% identical to SEQ ID NO: 28 (Table 3 above).

The targeting component may further comprise a light chain variable region, where said light chain variable region includes: a complementarity-determining region 1 (CDR-L1) having an amino acid sequence of SEQ ID NO: 19, or a modified amino acid sequence of SEQ ID NO: 19, said modified sequence having at least 80% sequence identity to SEQ ID NO: 19; a complementarity-determining region 2 (CDR-L2) having an amino acid sequence of SEQ ID NO: 22, or a modified amino acid sequence of SEQ ID NO: 22, said modified sequence having at least 80% sequence identity to SEQ ID NO: 22; and a complementarity-determining region 3 (CDR-L3) having an amino acid sequence of SEQ ID NO: 25, or a modified amino acid sequence of SEQ ID NO: 25, said modified sequence having at least 80% sequence identity to SEQ ID NO: 25. The sequences of the light chain CDR sequences are provided in Table 2 above.

In some embodiments, the light chain variable region includes an amino acid sequence that is at least 80% identical to SEQ ID NO: 29 (Table 3 above).

In some embodiments, the targeting component comprises a heavy chain variable region including the CDR-H1 of SEQ ID NO: 10, the CDR-H2 of SEQ ID NO: 13, and the CDR-H3 of SEQ ID NO: 16, and a light chain variable region including the CDR-L1 of SEQ ID NO: 19, the CDR-L2 of SEQ ID NO: 22, and the CDR-L3 of SEQ ID NO: 25.

In some embodiments, the targeting component comprises a heavy chain variable region including an amino acid sequence that is at least 80% identical to SEQ ID NO: 28 and a light chain variable region including an amino acid sequence that is at least 80% identical to SEQ ID NO: 29 (Table 3).

In some embodiments, the targeting component further includes a signaling peptide, optionally where the signaling peptide has the sequence of amino acids 1-19 of SEQ ID NO: 34.

In some embodiments, the glycosyltransferase is selected from the group consisting of glycosyltransferase A (Alpha 1-3-N-Acetylgalactosaminyltransferase) and glycosyltransferase B (alpha 1-3-galactosyltransferase).

In some embodiments, the glycosyltransferase is glycosyltransferase A (“GTA”) and has an amino acid sequence of SEQ ID NO: 64, or a portion thereof, as follows:

EPDHLQRVSLPRMVYPQPKVLTPCRKDVLVVTPWLAPIVWEGTFNIDILN
EQFRLQNTTIGLTVFAIKKYVAFLKLFLETAEKHFMVGHRVHYYVFTDQP
AAVPRVTLGTGRQLSVLEVRAYKRWQDVSMRRMEMISDFCERRFLSEVDY
LVCVDVDMEFRDHVGVEILTPLFGTLHPGFYGSSREAFTYERRPQSQAYI
PKDEGDFYYLGGFFGGSVQEVQRLTRACHQAMMVDQANGIEAVWHDESHL
NKYLLRHKPTKVLSPEYLWDQQLLGWPAVLRKLRFTAVPKNHQAVRNP.

In some embodiments, the glycosyltransferase is glycosyltransferase B (“GTB”) and has an amino acid sequence of SEQ ID NO: 65, or a portion thereof, as follows:

EPDHLQRVSLPRMVYPQPKVLTPCRKDVLVVTPWLAPIVWEGTFNIDILN
EQFRLQNTTIGLTVFAIKKYVAFLKLFLETAEKHFMVGHRVHYYVFTDQP
AAVPRVTLGTGRQLSVLEVGAYKRWQDVSMRRMEMISDFCERRFLSEVDY
LVCVDVDMEFRDHVGVEILTPLFGTLHPSFYGSSREAFTYERRPQSQAYI
PKDEGDFYYMGAFFGGSVQEVQRLTRACHQAMMVDQANGIEAVWHDESHL
NKYLLRHKPTKVLSPEYLWDQQLLGWPAVLRKLRFTAVPKNHQAVRN.

Suitable additional glycosylases are described infra. In some embodiments, the glycosyltransferase is Marmoset α-1,3 galactosyltransferase (aa90-376) and has an amino acid sequence of SEQ ID NO: 66, or a portion thereof, as follows:

ELRLWDWFNPKKRPEVMTVTQWKAPVVWEGTYNKAILENYYAKQKITVGL
TVFAIGRYIEHYLEEFVTSANRYFMVGHKVIFYVMVDDVSKAPFIELGPL
RSFKVFEVKPEKRWQDISMMRMKTIGEHILAHIQHEVDFLFCMDVDQVFQ
DHFGVETLGQSVAQLQAWWYKADPDDFTYERRKESAAYIPFGQGDFYYHA
AIFGGTPIQVLNITQECFKGILLDKKNDIEAEWHDESHLNKYFLLNKPSK
ILSPEYCWDYHIGLPSDIKTVKLSWQTKEYNLVRKNVGGGS.

In some embodiments, the bi-functional therapeutic includes: (i) a first protein comprising the amino acid sequence of SEQ ID NO: 34 or SEQ ID NO: 35 and a second protein comprising the amino acid sequence of SEQ ID NO: 36; (ii) a first protein comprising the amino acid sequence of SEQ ID NO: 37 or SEQ ID NO: 38 and a second protein comprising the amino acid sequence of SEQ ID NO: 39; (iii) a first protein comprising the amino acid sequence of SEQ ID NO: 40 or SEQ ID NO: 41 and a second protein comprising the amino acid sequence of SEQ ID NO: 42; (iv) a first protein comprising the amino acid sequence of SEQ ID NO: 43 or SEQ ID NO: 44 and a second protein comprising the amino acid sequence of SEQ ID NO: 45; (v) the amino acid sequence of SEQ ID NO: 46; (vi) the amino acid sequence of SEQ ID NO: 47; (vii) the amino acid sequence of SEQ ID NO: 48; or (viii) the amino acid sequence of SEQ ID NO: 49 (Table 4).

TABLE 4
J591 Bi-Functional Therapeutic Protein Sequences

| | | |
|---|---|---|
| Protein | | SEQ ID |
| Sequence | Sequence | NO: |

huJ591-GTB and huJ591-GTA Protein Sequences

| | | |
|---|---|---|
| Signal | MGWSCIILFLVATATGVHSEVQLVQSGPEVKKPGATVKISCKTSGYTFTEYTIH | 34 |
| peptide- | WVKQAPGKGLEWIGNINPNNGGTTYNQKFEDKATLTVDKSTDTAYMELSSLR | |
| huJ591 H | SEDTAVYYCAAGWNFDYWGQGTLLTVSSASTKGPSVFPLAPSSKSTSGGTAA | |
| chain-GTB | LGCLVKDYFPEPVTVSWNSGALTSGVHTFPAVLQSSGLYSLSSWTVPSSSLG | |
| | TQTYICNVNHKPSNTKVDKKVEPKSCDKTHTCPPCPAPELLGGPSVFLFPPKP | |
| | KDTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGKggggsggggsg | |
| | gggsEPDHLQRVSLPRMVYPQPKVLTPCRKDVLVVTPWLAPIVWEGTFNIDIL | |
| | NEQFRLQNTTIGLTVFAIKKYVAFLKLFLETAEKHFMVGHRVHYYVFTDQPAA | |
| | VPRVTLGTGRQLSVLEVGAYKRWQDVSMRRMEMISDFCERRFLSEVDYLVC | |
| | VDVDMEFRDHVGVEILTPLFGTLHPSFYGSSREAFTYERRPQSQAYIPKDEG | |
| | DFYYMGAFFGGSVQEVQRLTRACHQAMMVDQANGIEAVWHDESHLNKYLL | |
| | RHKPTKVLSPEYLWDQQLLGWPAVLRKLRFTAVPKNHQAVRNP | |
| | (Signal peptide sequence shown in italic; huJ591 H chain sequence shown in | |
| | double underline; linker sequence shown in lowercase; GTB sequence shown in | |
| | bold.) | |
| Signal | MGWSCIILFLVATATGVHSEVQLVQSGPEVKKPGATVKISCKTSGYTFTEYTIH | 35 |
| peptide- | WVKQAPGKGLEWIGNINPNNGGTTYNQKFEDKATLTVDKSTDTAYMELSSLR | |
| huJ591 H | SEDTAVYYCAAGWNFDYWGQGTLLTVSSASTKGPSVFPLAPSSKSTSGGTAA | |
| chain-GTA | LGCLVKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLFPPKP | |
| | KDTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGKggggsggggsg | |
| | gggsEPDHLQRVSLPRMVYPQPKVLTPCRKDVLVVTPWLAPIVWEGTFNIDIL | |
| | NEQFRLQNTTIGLTVFAIKKYVAFLKLFLETAEKHFMVGHRVHYYVFTDQPAA | |
| | VPRVTLGTGRQLSVLEVRAYKRWQDVSMRRMEMISDFCERRFLSEVDYLVC | |
| | VDVDMEFRDHVGVEILTPLFGTLHPGFYGSSREAFTYERRPQSQAYIPKDEG | |
| | DFYYLGGFFGGSVQEVQRLTRACHQAMMVDQANGIEAVWHDESHLNKYLL | |
| | RHKPTKVLSPEYLWDQQLLGWPAVLRKLRFTAVPKNHQAVRNP | |
| | (Signal peptide sequence shown in italic; huJ591 H chain sequence shown in | |
| | double underline; linker sequence shown in lowercase; GTA sequence shown in | |
| | bold.) | |
| Signal | MGWSCIILFLVATATGVHSDIQMTQSPSSLSTSVGDRVTLTCKASQDVGTAVD | 36 |
| peptide- | WYQQKPGPSPKLLIYWASTRHTGIPSRFSGSGSGTDFTLTISSLQPEDFADYY | |
| huJ591-LC- | CQQYNSYPLTFGPGTKVDIKRTVAAPSVFIFPPSDEQLKSGTASVVCLLNNFYP | |
| his tag | REAKVQWKVDNALQSGNSQESVTEQDSKDSTYSLSSTLTLSKADYEKHKVYA | |
| | CEVTHQGLSSPVTKSFNRGEC [symbol] | |
| | (Signal peptide sequence shown in italic; huJ591 L chain sequence shown in | |
| | double underline; his tag sequence shown in bold italic.) | |

huJ591Fab-GTB and huJ591Fab-GTA Protein Sequences

| | | |
|---|---|---|
| Signal | MGWSCIILFLVATATGVHSEVQLVQSGPEVKKPGATVKISCKTSGYTFTEYTIH | 37 |
| peptide- | WVKQAPGKGLEWIGNINPNNGGTTYNQKFEDKATLTVDKSTDTAYMELSSLR | |
| huJ591Fab- | SEDTAVYYCAAGWNFDYWGQGTLLTVSSASTKGPSVFPLAPSSKSTSGGTAA | |
| GTB-Myc- | LGCLVKDYFPEPVTVSWNSGALTSGVHTFPAVLQSSGLYSLSSWTVPSSSLG | |
| his tag | TQTYICNVNHKPSNTKVDKKVEPKSCDKTHTEPDHLQRVSLPRMVYPQPKVL | |
| | TPCRKDVLVVTPWLAPIVWEGTFNIDILNEQFRLQNTTIGLTVFAIKKYVAFLK | |
| | LFLETAEKHFMVGHRVHYYVFTDQPAAVPRVTLGTGRQLSVLEVGAYKRWQ | |
| | DVSMRRMEMISDFCERRFLSEVDYLVCVDVDMEFRDHVGVEILTPLFGTLHP | |
| | SFYGSSREAFTYERRPQSQAYIPKDEGDFYYMGAFFGGSVQEVQRLTRACH | |
| | QAMMVDQANGIEAVWHDESHLNKYLLRHKPTKVLSPEYLWDQQLLGWPAV | |
| | LRKLRFTAVPKNHQAVRNP [symbol] [symbol] | |
| | (Signal peptide sequence shown in italic; huJ591 Fab sequence shown in | |
| | double underline; GTB sequence shown in bold; Myc sequence shown in italic | |
| | double underline; his tag sequence shown in bold italic.) | |
| Signal | MGWSCIILFLVATATGVHSEVQLVQSGPEVKKPGATVKISCKTSGYTFTEYTIH | 38 |
| peptide- | WVKQAPGKGLEWIGNINPNNGGTTYNQKFEDKATLTVDKSTDTAYMELSSLR | |
| huJ591Fab- | SEDTAVYYCAAGWNFDYWGQGTLLTVSSASTKGPSVFPLAPSSKSTSGGTAA | |
| GTA-Myc- | LGCLVKDYFPEPVTVSWNSGALTSGVHTFPAVLQSSGLYSLSSVVTVPSSSLG | |
| his tag | TQTYICNVNHKPSNTKVDKKVEPKSCDKTHTEPDHLQRVSLPRMVYPQPKVL | |
| | TPCRKDVLVVTPWLAPIVWEGTFNIDILNEQFRLQNTTIGLTVFAIKKYVAFLK | |
| | LFLETAEKHFMVGHRVHYYVFTDQPAAVPRVTLGTGRQLSVLEVRAYKRWQ | |
| | DVSMRRMEMISDFCERRFLSEVDYLVCVDVDMEFRDHVGVEILTPLFGTLHP | |
| | GFYGSSREAFTYERRPQSQAYIPKDEGDFYYLGGFFGGSVQEVQRLTRACH | |
| | QAMMVDQANGIEAVWHDESHLNKYLLRHKPTKVLSPEYLWDQQLLGWPAV | |
| | LRKLRFTAVPKNHQAVRNP [symbol] [symbol] | |
| | (Signal peptide sequence shown in italic; huJ591 Fab sequence shown in | |
| | double underline; GTA sequence shown in bold; Myc sequence shown in italic | |
| | double underline; his tag sequence shown in bold italic.) | |
| Signal | MGWSCIILFLVATATGVHSDIQMTQSPSSLSTSVGEDRVTLTCKASQDVGTAVD | 39 |
| peptide- | WYQQKPGPSPKLLIYWASTRHTGIPSRESGSGSGTDFTLTISSLQPEDFADYY | |
| huJ591-LC | CQQYNSYPLTFGPGTKVDIKRTVAAPSVFIFPPSDEQLKSGTASWVCLLNNFYP | |
| | REAKVQWKVDNALQSGNSQESVTEQDSKDSTYSLSSTLTLSKADYEKHKVYA | |
| | CEVTHQGLSSPVTKSNRGEC | |
| | (Signal peptide sequence shown in italic; huJ591 LC sequence shown in double | |
| | underline.) | |

huJ591-HC67-GTB and huJ591-HC67-GTA Protein Sequences

| | | |
|---|---|---|
| huJ591- | EVQLVQSGPEVKKPGATVKISCKTSGYTFTEYTIHWVKQAPGKGLEWIGNINP | 40 |
| HC67-GTB | NNGGTTYNQKFEDKATLTVDKSTDTAYMELSSLRSEDTAVYYCAAGWNFDY | |
| | WGQGTLLTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTKV | |
| | DKKVEPKSCDKTHT [symbol] PP [symbol] PAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYT [symbol] PPSRDELTKNQVSL [symbol] CL | |
| | VKGFYPSDIAVEWESNGQPENNYKTTVPVLDSDGSFRLASYLTVDKSRWQQ | |
| | GNVFSCSVMHEALHNHYTQKSLSLSPGKggggsggggggggsEPDHLQRVSLPR | |
| | MVYPQPKVLTPCRKDVLVVTPWLAPIVWEGTFNIDILNEQFRLQNTTIGLTVF | |
| | AIKKYVAFLKLFLETAEKHFMVGHRVHYYVFTDQPAAVPRVTLGTGRQLSVL | |
| | EVGAYKRWQDVSMRRMEMISDFCERRFLSEVDYLVCVDVDMEFRDHVGVEI | |
| | LTPLFGTLHPSFYGSSREAFTYERRPQSQAYIPKDEGDFYYMGAFFGGSVQE | |
| | VQRLTRACHQAMMVDQANGIEAVWHDESHLNKYLLRHKPTKVLSPEYLWD | |
| | QQLLGWPAVLRKLRFTAVPKNHQAVRNP | |
| | (huJ591-HC67 sequence shown in double underline; linker sequence shown in | |
| | lowercase; GTB sequence shown in bold.) HC67 variant amino acids (n = 8) | |
| | shown in bold double underline. | |
| huJ591- | EVQLVQSGPEVKKPGATVKISCKTSGYTFTEYTIHWVKQAPGKGLEWIGNINP | 41 |
| HC67-GTA | NNGGTTYNQKFEDKATLTVDKSTDTAYMELSSLRSEDTAVYYCAAGWNFDY | |
| | WGQGTLLTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTKV | |
| | DKKVEPKSCDKTHT [symbol] PP [symbol] PAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTKPPSRDELTKNQVSLSCL | |
| | VKGFYPSDIAVEWESNGQPENNYKTTVPVLDSDGSFRLASYLTVDKSRWQQ | |
| | GNVFSCSVMHEALHNHYTQKSLSLSPGKggggggggsggggsEPDHLQRVSLPR | |
| | MVYPQPKVLTPCRKDVLVVTPWLAPIVWEGTFNIDILNEQFRLQNTTIGLTVF | |
| | AIKKYVAFLKLFLETAEKHFMVGHRVHYYVFTDQPAAVPRVTLGTGRQLSVL | |
| | EVRAYKRWQDVSMRRMEMISDFCERRFLSEVDYLVCVDVDMEFRDHVGVEI | |
| | LTPLFGTLHPGFYGSSREAFTYERRPQSQAYIPKDEGDFYYLGGFFGGSVQE | |
| | VQRLTRACHQAMMVDQANGIEAVWHDESHLNKYLLRHKPTKVLSPEYLWD | |
| | QQLLGWPAVLRKLRFTAVPKNHQAVRNP | |
| | (huJ591-HC67 sequence shown in double underline; linker sequence shown in | |
| | lowercase; GTA sequence shown in bold.) HC67 variant amino acids (n = 8) | |
| | shown in bold double underline. | |
| huJ591-LC- | DIQMTQSPSSLSTSVGDRVTLTCKASQDVGTAVDWYQQKPGPSPKLLIYWAS | 42 |
| his tag | TRHTGIPSRFSGSGSGTDFTLTISSLQPEDFADYYCQQYNSYPLTFGPGTKVDI | |
| | KRTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSFNR | |
| | GEC [symbol] | |
| | (huJ591 LC sequence shown in double underline; his tag sequence shown in | |
| | bold italic.) | |

huJ591-HC67-GTB-54aa and huJ591-HC67-GTA-54aa Protein Sequences

| | | |
|---|---|---|
| huJ591 | MNFGLRLIFLVLTLKGVQCEVQLVQSGPEVKKPGATVKISCKTSGYTFTEYTIH | 43 |
| HC67 - | WVKQAPGKGLEWIGNINPNNGGTTYNQKFEDKATLTVDKSTDTAYMELSSLR | |
| GTB-54aa | SEDTAVYYCAAGWNFDYWGQGTLLTVSSASTKGPSVFPLAPSSKSTSGGTAA | |
| | LGCLVKDYFPEPVTVSWNSGALTSGVHTFPAVLQSSGLYSLSSVVTVPSSSLG | |
| | TQTYICNVNHKPSNTKVDKKVEPKSCDKTHTVPPVPAPELLGGPSVFLFPPKP | |
| | KDTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYTKP | |
| | PSRDELTKNQVSLSCLVKGFYPSDIAVEWESNGQPENNYKTTVPVLDSDGSE | |
| | [symbol] L [symbol] S [symbol] LTVDKSRWQQGNVFSCSVMHEALHNHYTQKSLSLSPGKggggsggggs | |
| | ggggsEPDHLQRVSLPRMVYPQPKVLTPCRKDVLVVTPWLAPIVWEGTFNIDIL | |
| | NEQFRLQNTTIGLTVFAIKKYVAFLKLFLETAEKHFMVGHRVHYYVFTDQPAA | |
| | VPRVTLGTGRQLSVLEVGAYKRWQDVSMRRMEMISDFCERRFLSEVDYLVC | |
| | VDVDMEFRDHVGVEILTPLFGTLHPSFYGSSREAFTYERRPQSQAYIPKDEG | |
| | DFYYMGAFFGGSVQEVQRLTRACHQAMMVDQANGIEAVWHDESHLNKYLL | |
| | RHKPTKVLSPEYLWDQQLLGWPAVLRKLRFTAVPKNHQAVRNP [symbol] | |
| | [symbol] | |
| | (Signal peptide sequence shown in italic; huJ591 H chain sequence shown in | |
| | double underline; linker sequence shown in lowercase; GTB sequence shown in | |
| | bold; 54aa sequence shown in italic double underline.) HC67 variant amino | |
| | acids (n = 8) shown in bold double underline. | |
| huJ591- | MNFGLRLIFLVLTLKGVQCEVQLVQSGPEVKKPGATVKISCKTSGYTFTEYTIH | 44 |
| HC67-GTA- | WVKQAPGKGLEWIGNINPNNGGTTYNQKFEDKATLTVDKSTDTAYMELSSLR | |
| 54aa | SEDTAVYYCAAGWNFDYWGQGTLLTVSSASTKGPSVFPLAPSSKSTSGGTAA | |
| | LGCLVKDYFPEPVTVSWNSGALTSGVHTFPAVLQSSGLYSLSSWVTVPSSSLG | |
| | TQTYICNVNHKPSNTKVDKKVEPKSCDKTHT [symbol] PP [symbol] PAPELLGGPSVFLFPPKP | |
| | KDTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYT [symbol] P | |
| | PSRDELTKNQVSLSCLVKGFYPSDIAVEWESNGQPENNYKTTVPVLDSDGSF | |
| | [symbol] L [symbol] S [symbol] LTVDKSRWQQGNVFSCSVMHEALHNHYTQKSLSLSPGKggggggggs | |
| | ggggsEPDHLQRVSLPRMVYPQPKVLTPCRKDVLVVTPWLAPIVWEGTFNIDIL | |
| | NEQFRLQNTTIGLTVFAIKKYVAFLKLFLETAEKHFMVGHRVHYYVFTDQPAA | |
| | VPRVTLGTGRQLSVLEVRAYKRWQDVSMRRMEMISDFCERRFLSEVDYLVC | |
| | VDVDMEFRDHVGVEILTPLFGTLHPGFYGSSREAFTYERRPQSQAYIPKDEG | |
| | DFYYLGGFFGGSVQEVQRLTRACHQAMMVDQANGIEAVWHDESHLNKYLL | |
| | RHKPTKVLSPEYLWDQQLLGWPAVLRKLRFTAVPKNHQAVRNP [symbol] | |
| | [symbol] | |
| | (Signal peptide sequence shown in italic; huJ591 H chain sequence shown in | |
| | double underline; linker sequence shown in lowercase; GTA sequence shown in | |
| | bold; 54aa sequence shown in italic double underline.) HC67 variant amino | |
| | acids (n = 8) shown in bold double underline. | |
| huJ591-LC- | DIQMTQSPSSLSTSVGDRVTLTCKASQDVGTAVDWYQQKPGPSPKLLIYWAS | 45 |
| his tag | TRHTGIPSRFSGSGSGTDFTLTISSLQPEDFADYYCQQYNSYPLTFGPGTKVDI | |
| | KRTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSFNR | |
| | GEC [symbol] | |
| | (huJ591 LC sequence shown in double underline; his tag sequence shown in | |
| | bold italic.) | |

huJ591scFv-Fc67-GTB and huJ591scFv-Fc67-GTA Protein Sequences

| | | |
|---|---|---|
| huJ591scFv- | DIQMTQSPSSLSTSVGDRVTLTCKASQDVGTAVDWYQQKPGPSPKLLIYWAS | 46 |
| Fc67- | TRHTGIPSRESGSGSGTDFTLTISSLQPEDFADYYCQQYNSYPLTFGPGTKVDI | |
| GTB-his | KEVQLVQSGPEVKKPGATVKISCKTSGYTFTEYTIHWVKQAPGKGLEWIGNIN | |
| tag | PNNGGTTYNQKFEDKATLTVDKSTDTAYMELSSLRSEDTAVYYCAAGWNFDY | |
| | WGQGTLLTVSSEPKSCDKTHT [symbol] PP [symbol] PAPELLGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALPAPIEKTISKAKGQPREPQVYT [symbol] PPSRDELTKN | |
| | QVSLSCLVKGFYPSDIAVEWESNGQPENNYKTTVPVLDSDGSFRLAS [symbol] LTVD | |
| | KSRWQQGNVFSCSVMHEALHNHYTQKSLSLSPGKggggsggggsggggsEPDHL | |
| | QRVSLPRMVYPQPKVLTPCRKDVLVVTPWLAPIVWEGTFNIDILNEQFRLQN | |
| | TTIGLTVFAIKKYVAFLKLFLETAEKHFMVGHRVHYYVFTDQPAAVPRVTLGT | |
| | GRQLSVLEVGAYKRWQDVSMRRMEMISDFCERRFLSEVDYLVCVDVDMEF | |
| | RDHVGVEILTPLFGTLHPSFYGSSREAFTYERRPQSQAYIPKDEGDFYYMGA | |
| | FFGGSVQEVQRLTRACHQAMMVDQANGIEAVWHDESHLNKYLLRHKPTKV | |
| | LSPEYLWDQQLLGWPAVLRKLRFTAVPKNHQAVRNP [symbol] | |
| | (huJ591scFv-Fc67 sequence shown in double underline; linker sequence | |
| | shown in lowercase; GTB sequence shown in bold; his tag sequence shown in | |
| | bold italic.) HC67 variant amino acids (n = 8) shown in bold double underline. | |
| h591scFv- | DIQMTQSPSSLSTSVGDRVTLTCKASQDVGTAVDWYQQKPGPSPKLLIYWAS | 47 |
| Fc67-GTA- | TRHTGIPSRFSGSGSGTDFTLTISSLQPEDFADYYCQQYNSYPLTFGPGTKVDI | |
| his tag | KEVQLVQSGPEVKKPGATVKISCKTSGYTFTEYTIHWVKQAPGKGLEWIGNIN | |
| | PNNGGTTYNQKFEDKATLTVDKSTDTAYMELSSLRSEDTAVYYCAAGWNFDY | |
| | WGQGTLLTVSSEPKSCDKTHT [symbol] PP [symbol] PAPELLGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALPAPIEKTISKAKGQPREPQVYT [symbol] PPSRDELTKN | |
| | QVSL [symbol] CLVKGFYPSDIAVEWESNGQPENNYKTT [symbol] PVLDSDGSF [symbol] L [symbol] S [symbol] LTVD | |
| | KSRWQQGNVFSCSVMHEALHNHYTQKSLSLSPGKggggsggggsggggsEPDHL | |
| | QRVSLPRMVYPQPKVLTPCRKDVLVVTPWLAPIVWEGTFNIDILNEQFRLQN | |
| | TTIGLTVFAIKKYVAFLKLFLETAEKHFMVGHRVHYYVFTDQPAAVPRVTLGT | |
| | GRQLSVLEVRAYKRWQDVSMRRMEMISDFCERRFLSEVDYLVCVDVDMEFR | |
| | DHVGVEILTPLFGTLHPGFYGSSREAFTYERRPQSQAYIPKDEGDFYYLGGFF | |
| | GGSVQEVQRLTRACHQAMMVDQANGIEAVWHDESHLNKYLLRHKPTKVLS | |
| | PEYLWDQQLLGWPAVLRKLRFTAVPKNHQAVRNP [symbol] | |
| | (huJ591scFv-Fc67 sequence shown in double underline; linker sequence | |
| | shown in lowercase; GTA sequence shown in bold; his tag sequence shown in | |
| | bold italic.) HC67 variant amino acids (n = 8) shown in bold double underline. | |

huJ591scFv-GTB and huJ591scFv-GTA Protein Sequences

| | | |
|---|---|---|
| huJ591scFv- | METDTLLLWVLLLWVPGSTGEVQLVQSGAEVKKPGASVKISCKTSGYTFTEYT | 48 |
| GTB- | IHWVKQASGKGLEWIGNINPNNGGTTYNQKFEDRATLTVDKSTSTAYMELSSL | |
| Myc-His tag | RSEDTAVYYCAAGWNFDYWGQGTTVTVSSGSTSGGGSGGGSGGGGSSDIV | |
| | MTQSPSSLSASVGDRVTITCKASQDVGTAVDWYQQKPGKAPKLLIYWASTRH | |
| | TGVPDRFTGSGSGTDFTLTISSLQPEDFADYFCQQYNSYPLTFGGGTKLEIKgg | |
| | ggsggggsggggsEPDHLQRVSLPRMVYPQPKVLTPCRKDVLVVTPWLAPIVWE | |
| | GTFNIDILNEQFRLQNTTIGLTVFAIKKYVAFLKLFLETAEKHFMVGHRVHYYV | |
| | FTDQPAAVPRVTLGTGRQLSVLEVGAYKRWQDVSMRRMEMISDFCERRFLS | |
| | EVDYLVCVDVDMEFRDHVGVEILTPLFGTLHPSFYGSSREAFTYERRPQSQA | |
| | YIPKDEGDFYYMGAFFGGSVQEVQRLTRACHQAMMVDQANGIEAVWHDES | |
| | HLNKYLLRHKPTKVLSPEYLWDQQLLGWPAVLRKLRFTAVPKNHQAVRNP [symbol] | |
| | [symbol] [symbol] | |
| | (Signal peptide sequence shown in italic; huJ591scFv sequence shown in | |
| | double underline; linker sequence shown in lowercase; GTB sequence shown in | |
| | bold; Myc sequence shown in italic double underline; his tag sequence shown in | |
| | bold italic.) | |
| huJ591scFv- | METDTLLLWVLLLWVPGSTGEVQLVQSGAEVKKPGASVKISCKTSGYTFTEYT | 49 |
| GTA- | IHWVKQASGKGLEWIGNINPNNGGTTYNQKFEDRATLTVDKSTSTAYMELSSL | |
| Myc-His tag | RSEDTAVYYCAAGWNFDYWGQGTTVTVSSGSTSGGGSGGGSGGGGSSDIV | |
| | MTQSPSSLSASVGDRVTITCKASQDVGTAVDWYQQKPGKAPKLLIYWASTRH | |
| | TGVPDRFTGSGSGTDFTLTISSLQPEDFADYFCQQYNSYPLTFGGGTKLEIKgg | |
| | ggsggggsggggsEPDHLQRVSLPRMVYPQPKVLTPCRKDVLVVTPWLAPIVWE | |
| | GTFNIDILNEQFRLQNTTIGLTVFAIKKYVAFLKLFLETAEKHFMVGHRVHYYV | |
| | FTDQPAAVPRVTLGTGRQLSVLEVRAYKRWQDVSMRRMEMISDFCERRFLS | |
| | EVDYLVCVDVDMEFRDHVGVEILTPLFGTLHPGFYGSSREAFTYERRPQSQA | |
| | YIPKDEGDFYYLGGFFGGSVQEVQRLTRACHQAMMVDQANGIEAVWHDES | |
| | HLNKYLLRHKPTKVLSPEYLWDQQLLGWPAVLRKLRFTAVPKNHQAVRNP [symbol] | |
| | [symbol] [symbol] | |
| | (Signal peptide sequence shown in italic; huJ591scFv sequence shown in | |
| | double underline; linker sequence shown in lowercase; GTA sequence shown in | |
| | bold; Myc sequence shown in italic double underline; his tag sequence shown in | |
| | bold italic.) | |

In some embodiments, the bi-functional therapeutic includes a first protein comprising the amino acid sequence of positions 20-770 of SEQ ID NO: 34 or SEQ ID NO: 35 and a second protein comprising the amino acid sequence of positions 20-233 of SEQ ID NO: 36.

In some embodiments, the bi-functional therapeutic includes a first protein comprising the amino acid sequence of positions 20-540 of SEQ ID NO: 37 or SEQ ID NO: 38 and a second protein comprising the amino acid sequence of positions 20-233 of SEQ ID NO: 39, optionally where the bi-functional therapeutic comprises a first portion comprising the amino acid sequence of positions 20-558 of SEQ ID NO: 37 or SEQ ID NO: 38.

In some embodiments, the bi-functional therapeutic includes a first protein comprising the amino acid sequence of SEQ ID NO: 40 or SEQ ID NO: 41 and a second protein comprising the amino acid sequence of positions 1-214 of SEQ ID NO: 42.

In some embodiments, the bi-functional therapeutic includes a first protein comprising the amino acid sequence of positions 20-831 of SEQ ID NO: 43 or SEQ ID NO: 44 and a second protein comprising the amino acid sequence of positions 1-214 of SEQ ID NO: 45.

In some embodiments, the bi-functional therapeutic includes the amino acid sequence of positions 1-767 of SEQ ID NO: 46 or SEQ ID NO: 47.

In some embodiments, the bi-functional therapeutic includes the amino acid sequence of positions 20-591 of SEQ ID NO: 48 or SEQ ID NO: 49, optionally where the bi-functional therapeutic comprises the sequence of positions 20-597 of SEQ ID NO: 37 or SEQ ID NO: 38.

Another aspect of the present disclosure relates to a bi-functional therapeutic for treating cancer that includes a targeting component which targets a human epidermal growth factor receptor (HER) family member and a glycosyltransferase which, when delivered to a tumor by said targeting component, enzymatically converts the tumor phenotype to that of an incompatible allograft or xenograft, said glycosyltransferase being coupled to said targeting component. This aspect of the present disclosure is useful in treating a subject with breast cancer or any HER2 expressing cancer.

In some embodiments, the targeting component comprises a heavy chain variable region, where said heavy chain variable region includes: a complementarity-determining region 1 (CDR-H1) comprising an amino acid sequence of SEQ ID NO: 11, or a modified amino acid sequence of SEQ ID NO: 11, said modified sequence having at least 80% sequence identity to SEQ ID NO: 11; a complementarity-determining region 2 (CDR-H2) comprising an amino acid sequence of SEQ ID NO: 14, or a modified amino acid sequence of SEQ ID NO: 14, said modified sequence having at least 80% sequence identity to SEQ ID NO: 14; and a complementarity-determining region 3 (CDR-H3) comprising an amino acid sequence of SEQ ID NO: 17, or a modified amino acid sequence of SEQ ID NO: 17, said modified sequence having at least 80% sequence identity to SEQ ID NO: 17. The sequences of the heavy chain CDR sequences are provided in Table 1 above.

In some embodiments, the targeting component comprises a heavy chain variable region including an amino acid sequence that is at least 80% identical to SEQ ID NO: 30 (Table 3 above).

The targeting component may further comprise a light chain variable region, where said light chain variable region includes: a complementarity-determining region 1 (CDR-L1) having an amino acid sequence of SEQ ID NO: 20 or a modified amino acid sequence of SEQ ID NO: 20, said modified sequence having at least 80% sequence identity to SEQ ID NO: 20; a complementarity-determining region 2 (CDR-L2) having an amino acid sequence of SEQ ID NO: 23, or a modified amino acid sequence of SEQ ID NO: 23, said modified sequence having at least 80% sequence identity to SEQ ID NO: 23; and a complementarity-determining region 3 (CDR-L3) having an amino acid sequence of SEQ ID NO: 26, or a modified amino acid sequence of SEQ ID NO: 26, said modified sequence having at least 80% sequence identity to SEQ ID NO: 26. The sequences of the light chain CDR sequences are provided in Table 2 above.

In some embodiments, the light chain variable region includes an amino acid sequence that is at least 80% identical to SEQ ID NO: 31 (Table 3 above).

In some embodiments, the targeting component comprises a heavy chain variable region including the CDR-H1 of SEQ ID NO: 11, the CDR-H2 of SEQ ID NO: 14, and the CDR-H3 of SEQ ID NO: 17, and a light chain variable region including the CDR-L1 of SEQ ID NO: 20, the CDR-L2 of SEQ ID NO: 23, and the CDR-L3 of SEQ ID NO: 26.

In some embodiments, the targeting component comprises a heavy chain variable region including an amino acid sequence that is at least 80% identical to SEQ ID NO: 30 and a light chain variable region including an amino acid sequence that is at least 80% identical to SEQ ID NO: 31 (Table 3).

In some embodiments, the targeting component further includes a signaling peptide, optionally where the signaling peptide has the sequence of amino acids 1-19 of SEQ ID NO: 50.

Suitable glycosyltransferases are described in detail infra.

In some embodiments, the glycosyltransferase is selected from the group consisting of glycosyltransferase A (Alpha 1-3-N-Acetylgalactosaminyltransferase) and glycosyltransferase B (alpha 1-3-gal actosyltransferase).

In some embodiments, the bi-functional therapeutic includes: (i) a first protein comprising the amino acid sequence of SEQ ID NO: 50 or SEQ ID NO: 51 and a second protein comprising the amino acid sequence of SEQ ID NO: 52, (ii) a first protein comprising the amino acid sequence of SEQ ID NO: 53 or SEQ ID NO: 54 and a second protein comprising the amino acid sequence of SEQ ID NO: 55; (iii) a first protein comprising the amino acid sequence of SEQ ID NO: 56 or SEQ ID NO: 57 and a second protein comprising the amino acid sequence of SEQ ID NO: 58; (iv) the amino acid sequence of SEQ ID NO: 59; (v) the amino acid sequence of SEQ ID NO: 60; (vi) the amino acid sequence of SEQ ID NO: 61; or (vii) the amino acid sequence of SEQ ID NO: 62 (Table 5).

TABLE 5
4D5 Bi-Functional Therapeutic Protein Sequences

| | | |
|---|---|---|
| Protein | | SEQ ID |
| Sequence | Sequence | NO: |

4D5-GTB and 4D5-GTA Protein Sequences

| | | |
|---|---|---|
| Signal | MGWSCIILFLVATATGVHSEVQLVESGGGLVQPGGSLRLSCAASGFNIKDTY | 50 |
| peptide-4D5 | IHWVRQAPGKGLEWVARIYPTNGYTRYADSVKGRFTISADTSKNTAYLQMN | |
| H chain-GTB | SLRAEDTAVYYCSRWGGDGFYAMDYWGQGTLVTVSSASTKGPSVFPLAPS | |
| | SKSTSGGTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCPPCPAPEL | |
| | LGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYTQ | |
| | KSLSLSPGggggsggggsggggsEPDHLQRVSLPRMVYPQPKVLTPCRKDVLVV | |
| | TPWLAPIVWEGTFNIDILNEQFRLQNTTIGLTVFAIKKYVAFLKLFLETAEKH | |
| | FMVGHRVHYYVFTDQPAAVPRVTLGTGRQLSVLEVGAYKRWQDVSMRRM | |
| | EMISDFCERRFLSEVDYLVCVDVDMEFRDHVGVEILTPLFGTLHPSFYGSSR | |
| | EAFTYERRPQSQAYIPKDEGDFYYMGAFFGGSVQEVQRLTRACHQAMMV | |
| | DQANGIEAVWHDESHLNKYLLRHKPTKVLSPEYLWDQQLLGWPAVLRKL | |
| | RFTAVPKNHQAVRNP | |
| | (Signal peptide sequence shown in italic; linker sequence shown in lowercase; | |
| | 4D5 H chain sequence shown in double underline; GTB sequence shown in | |
| | bold.) | |
| Signal | MGWSCIILFLVATATGVHSEVQLVESGGGLVQPGGSLRLSCAASGFNIKDTY | 51 |
| peptide-4D5 | IHWVRQAPGKGLEWVARIYPTNGYTRYADSVKGRFTISADTSKNTAYLQMN | |
| H chain -GTA | SLRAEDTAVYYCSRWGGDGFYAMDYWGQGTLVTVSSASTKGPSVFPLAPS | |
| | SKSTSGGTAALGCLVKDYFPEPVTVSWNSGALTSGVHTFPAVLQSSGLYSL | |
| | SSWVTVPSSSLGTQTYICNVNHKPSNTKVDKKVEPKSCDKTHTCPPCPAPEL | |
| | LGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYTQ | |
| | KSLSLSPGggggsggggggggsEPDHLQRVSLPRMVYPQPKVLTPCRKDVLVV | |
| | TPWLAPIVWEGTFNIDILNEQFRLQNTTIGLTVFAIKKYVAFLKLFLETAEKH | |
| | FMVGHRVHYYVFTDQPAAVPRVTLGTGRQLSVLEVRAYKRWQDVSMRRM | |
| | EMISDFCERRFLSEVDYLVCVDVDMEFRDHVGVEILTPLFGTLHPGFYGSS | |
| | REAFTYERRPQSQAYIPKDEGDFYYLGGFFGGSVQEVQRLTRACHQAMM | |
| | VDQANGIEAVWHDESHLNKYLLRHKPTKVLSPEYLWDQQLLGWPAVLRK | |
| | LRFTAVPKNHQAVRNP | |
| | (Signal peptide sequence shown in italic; linker sequence shown in lowercase; | |
| | 4D5 H chain sequence shown in double underline; GTA sequence shown in | |
| | bold.) | |
| Signal | MGWSCIILFLVATATGVHSDIQMTQSPSSLSASVGDRVTITCRASQDVNTAV | 52 |
| peptide- 4D5- | AWYQQKPGKAPKLLIYSASFLYSGVPSRFSGSRSGTDFTLTISSLQPEDFAT | |
| LC -his tag | YYCQQHYTTPPTFGQGTKVEIKRTVAAPSVFIFPPSDEQLKSGTASVVCLLN | |
| | NFYPREAKVQWKVDNALQSGNSQESVTEQDSKDSTYSLSSTLTLSKADYEK | |
| | HKVYACEVTHQGLSSPVTKSFNRGEC [symbol] | |
| | (Signal peptide sequence shown in italic; 4D5 L chain sequence shown in | |
| | double underline; his tag sequence shown in bold italic.) | |

4D5Fab-GTB and 4D5Fab-GTA Protein Sequences

| | | |
|---|---|---|
| Signal | MGWSCIILFLVATATGVHSEVQLVESGGGLVQPGGSLRLSCAASGFNIKDTY | 53 |
| peptide- | IHWVRQAPGKGLEWVARIYPTNGYTRYADSVKGRFTISADTSKNTAYLQMN | |
| 4D5Fab- | SLRAEDTAVYYCSRWGGDGFYAMDYWGQGTLVTVSSASTKGPSVFPLAPS | |
| GTB-Myc-his | SKSTSGGTAALGCLVKDYFPEPVTVSWNSGALTSGVHTFPAVLQSSGLYSL | |
| tag | SSVVTVPSSSLGTQTYICNVNHKPSNTKVDKKVEPKSCDKTHTEPDHLQRV | |
| | SLPRMVYPQPKVLTPCRKDVLVVTPWLAPIVWEGTFNIDILNEQFRLQNTTI | |
| | GLTVFAIKKYVAFLKLFLETAEKHFMVGHRVHYYVFTDQPAAVPRVTLGTG | |
| | RQLSVLEVGAYKRWQDVSMRRMEMISDFCERRFLSEVDYLVCVDVDMEF | |
| | RDHVGVEILTPLFGTLHPSFYGSSREAFTYERRPQSQAYIPKDEGDFYYMG | |
| | AFFGGSVQEVQRLTRACHQAMMVDQANGIEAVWHDESHLNKYLLRHKPT | |
| | KVLSPEYLWDQQLLGWPAVLRKLRFTAVPKNHQAVRNP [symbol] | |
| | [symbol] [symbol] | |
| | (Signal peptide sequence shown in italic; 4D5 Fab sequence shown in double | |
| | underline; GTB sequence shown in bold; Myc sequence shown in italic double | |
| | underline; his tag sequence shown in bold italic.) | |
| Signal | MGWSCIILFLVATATGVHSEVQLVESGGGLVQPGGSLRLSCAASGFNIKDTY | 54 |
| peptide- | IHWVRQAPGKGLEWVARIYPTNGYTRYADSVKGRFTISADTSKNTAYLQMN | |
| 4D5Fab- | SLRAEDTAVYYCSRWGGDGFYAMDYWGQGTLVTVSSASTKGPSVFPLAPS | |
| GTA-Myc-his | SKSTSGGTAALGCLVKDYFPEPVTVSWNSGALTSGVHTFPAVLQSSGLYSL | |
| tag | SSVVTVPSSSLGTQTYICNVNHKPSNTKVDKKVEPKSCDKTHTEPDHLQRV | |
| | SLPRMVYPQPKVLTPCRKDVLVVTPWLAPIVWEGTFNIDILNEQFRLQNTTI | |
| | GLTVFAIKKYVAFLKLFLETAEKHFMVGHRVHYYVFTDQPAAVPRVTLGTG | |
| | RQLSVLEVRAYKRWQDVSMRRMEMISDFCERRFLSEVDYLVCVDVDMEFR | |
| | DHVGVEILTPLFGTLHPGFYGSSREAFTYERRPQSQAYIPKDEGDFYYLGG | |
| | FFGGSVQEVQRLTRACHQAMMVDQANGIEAVWHDESHLNKYLLRHKPTK | |
| | VLSPEYLWDQQLLGWPAVLRKLRFTAVPKNHQAVRNP [symbol] | |
| | [symbol] [symbol] | |
| | (Signal peptide sequence shown in italic; 4D5 Fab sequence shown in double | |
| | underline; GTA sequence shown in bold; Myc sequence shown in italic double | |
| | underline; his tag sequence shown in bold italic.) | |
| Signal | MGWSCIILFLVATATGVHSDIQMTQSPSSLSASVGDRVTITCRASQDVNTAV | 55 |
| peptide- 4D5- | AWYQQKPGKAPKLLIYSASFLYSGVPSRFSGSRSGTDFTLTISSLQPEDFAT | |
| LC | YYCQQHYTTPPTFGQGTKVEIKRTVAAPSVFIFPPSDEQLKSGTASVVCLLN | |
| | NFYPREAKVQWKVDNALQSGNSQESVTEQDSKDSTYSLSSTLTLSKADYEK | |
| | HKVYACEVTHQGLSSPVTKSFNRGEC | |
| | (Signal peptide sequence shown in italic; 4D5 LC sequence shown in double | |
| | underline.) | |

4D5HC67-GTB and 4D5HC67-GTA Protein Sequences

| | | |
|---|---|---|
| 4D5HC67- | EVQLVESGGGLVQPGGSLRLSCAASGFNIKDTYIHWVRQAPGKGLEWVARI | 56 |
| GTB | YPTNGYTRYADSVKGRFTISADTSKNTAYLQMNSLRAEDTAVYYCSRWGGD | |
| | GFYAMDYWGQGTLVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNV | |
| | NHKPSNTKVDKKVEPKSCDKTHTggggsggggggggsEPDHLQRVSLPRMVY | |
| | PQPKVLTPCRKDVLVVTPWLAPIVWEGTFNIDILNEQFRLQNTTIGLTVFAIK | |
| | KYVAFLKLFLETAEKHFMVGHRVHYYVFTDQPAAVPRVTLGTGRQLSVLE | |
| | VGAYKRWQDVSMRRMEMISDFCERRFLSEVDYLVCVDVDMEFRDHVGVEI | |
| | LTPLFGTLHPSFYGSSREAFTYERRPQSQAYIPKDEGDFYYMGAFFGGSVQ | |
| | EVQRLTRACHQAMMVDQANGIEAVWHDESHLNKYLLRHKPTKVLSPEYL | |
| | WDQQLLGWPAVLRKLRFTAVPKNHQAVRNP | |
| | (4D5H67 sequence shown in double underline; linker sequence shown in | |
| | lowercase; GTB sequence shown in bold.) | |
| 4D5HC67- | EVQLVESGGGLVQPGGSLRLSCAASGFNIKDTYIHWVRQAPGKGLEWVARI | 57 |
| GTA | YPTNGYTRYADSVKGRFTISADTSKNTAYLQMNSLRAEDTAVYYCSRWGGD | |
| | GFYAMDYWGQGTLVTVSSASTKGPSVFPLAPSSKSTSGGTAALGCLVKDYE | |
| | PEPVTVSWNSGALTSGVHTFPAVLQSSGLYSLSSWVTVPSSSLGTQTYICNV | |
| | NHKPSNTKVDKKVEPKSCDKTHTggggsggggsggggsEPDHLQRVSLPRMVY | |
| | PQPKVLTPCRKDVLVVTPWLAPIVWEGTFNIDILNEQFRLQNTTIGLTVFAIK | |
| | KYVAFLKLFLETAEKHFMVGHRVHYYVFTDQPAAVPRVTLGTGRQLSVLE | |
| | VRAYKRWQDVSMRRMEMISDFCERRFLSEVDYLVCVDVDMEFRDHVGVEI | |
| | LTPLFGTLHPGFYGSSREAFTYERRPQSQAYIPKDEGDFYYLGGFFGGSVQ | |
| | EVQRLTRACHQAMMVDQANGIEAVWHDESHLNKYLLRHKPTKVLSPEYL | |
| | WDQQLLGWPAVLRKLRFTAVPKNHQAVRNP | |
| | (4D5H67 sequence shown in double underline; linker sequence shown in | |
| | lowercase; GTA sequence shown in bold.) | |
| 4D5-LC-his | DIQMTQSPSSLSASVGDRVTITCRASQDVNTAVAWYQQKPGKAPKLLIYSAS | 58 |
| tag | FLYSGVPSRFSGSRSGTDFTLTISSLQPEDFATYYCQQHYTTPPTFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTK | |
| | SFENRGEC [symbol] | |
| | (4D5 LC sequence shown in double underline; his tag sequence shown in | |
| | bold italic.) | |

4D5scFv-Fc67-GTB and 4D5scFv-Fc67-GTA Protein Sequences

| | | |
|---|---|---|
| 4D5scFv- | EVQLVESGGGLVQPGGSLRLSCAASGFNIKDTYIHWVRQAPGKGLEWVARI | 59 |
| Fc67-GTB- | YPTNGYTRYADSVKGRFTISADTSKNTAYLQMNSLRAEDTAVYYCSRWGGD | |
| his tag | GFYAMDYWGQGTLVTVSSGGGGSGGGGSGGGGSDIQMTQSPSSLSASVG | |
| | DRVTITCRASQDVNTAVAWYQQKPGKAPKLLIYSASFLYSGVPSRFSGSRS | |
| | GTDFTLTISSLQPEDFATYYCQQHYTTPPTFGQGTKVEIKggggsggggsggggs | |
| | EPDHLQRVSLPRMVYPQPKVLTPCRKDVLVVTPWLAPIVWEGTFNIDILNE | |
| | QFRLQNTTIGLTVFAIKKYVAFLKLFLETAEKHFMVGHRVHYYVFTDQPAAV | |
| | PRVTLGTGRQLSVLEVGAYKRWQDVSMRRMEMISDFCERRFLSEVDYLVC | |
| | VDVDMEFRDHVGVEILTPLFGTLHPSFYGSSREAFTYERRPQSQAYIPKDE | |
| | GDFYYMGAFFGGSVQEVQRLTRACHQAMMVDQANGIEAVWHDESHLNKY | |
| | LLRHKPTKVLSPEYLWDQQLLGWPAVLRKLRFTAVPKNHQAVRNP [symbol] | |
| | [symbol] | |
| | (4D5scFv-Fc67 sequence shown in double underline; linker sequence shown | |
| | in lowercase; GTB sequence shown in bold; his tag sequence shown in bold | |
| | italic.) | |
| 4D5scFv- | EVQLVESGGGLVQPGGSLRLSCAASGFNIKDTYIHWVRQAPGKGLEWVARI | 60 |
| Fc67-GTA- | YPTNGYTRYADSVKGRFTISADTSKNTAYLQMNSLRAEDTAVYYCSRWGGD | |
| his tag | GFYAMDYWGQGTLVTVSSGGGGSGGGGSGGGGSDIQMTQSPSSLSASVG | |
| | DRVTITCRASQDVNTAVAWYQQKPGKAPKLLIYSASFLYSGVPSRESGSRS | |
| | GTDFTLTISSLQPEDFATYYCQQHYTTPPTFGQGTKVEIKggggsggggsggggs | |
| | EPDHLQRVSLPRMVYPQPKVLTPCRKDVLVVTPWLAPIVWEGTFNIDILNE | |
| | QFRLQNTTIGLTVFAIKKYVAFLKLFLETAEKHFMVGHRVHYYVFTDQPAAV | |
| | PRVTLGTGRQLSVLEVRAYKRWQDVSMRRMEMISDFCERRFLSEVDYLVC | |
| | VDVDMEFRDHVGVEILTPLFGTLHPGFYGSSREAFTYERRPQSQAYIPKDE | |
| | GDFYYLGGFFGGSVQEVQRLTRACHQAMMVDQANGIEAVWHDESHLNKY | |
| | LLRHKPTKVLSPEYLWDQQLLGWPAVLRKLRFTAVPKNHQAVRNP [symbol] | |
| | [symbol] | |
| | (4D5scFv-Fc67 sequence shown in double underline; linker sequence shown | |
| | in lowercase; GTA sequence shown in bold; his tag sequence shown in bold | |
| | italic.) | |

4D5scFv-GTB and 4D5scFv-GTA Protein Sequences

| | | |
|---|---|---|
| 4D5scFv- | METDTLLLWVLLLWVPGSTGEVQLVESGGGLVQPGGSLRLSCAASGFNIKD | 61 |
| GTB-Myc-His | TYIHWVRQAPGKGLEWVARIYPTNGYTRYADSVKGRFTISADTSKNTAYLQ | |
| tag | MNSLRAEDTAVYYCSRWGGDGFYAMDYWGQGTLVTVSSGGGGSGGGGS | |
| | GGGGSDIQMTQSPSSLSASVGDRVTITCRASQDVNTAVAWYQQKPGKAPK | |
| | LLIYSASFLYSGVPSRFSGSRSGTDFTLTISSLQPEDFATYYCQQHYTTPPTF | |
| | GQGTKVEIKggggsggggsggggsEPDHLQRVSLPRMVYPQPKVLTPCRKDVLV | |
| | VTPWLAPIVWEGTFNIDILNEQFRLQNTTIGLTVFAIKKYVAFLKLFLETAEK | |
| | HFMVGHRVHYYVFTDQPAAVPRVTLGTGRQLSVLEVGAYKRWQDVSMRR | |
| | MEMISDFCERRFLSEVDYLVCVDVDMEFRDHVGVEILTPLFGTLHPSFYGS | |
| | SREAFTYERRPQSQAYIPKDEGDFYYMGAFFGGSVQEVQRLTRACHQAM | |
| | MVDQANGIEAVWHDESHLNKYLLRHKPTKVLSPEYLWDQQLLGWPAVLR | |
| | KLRFTAVPKNHQAVRNP [symbol] [symbol] | |
| | (Signal peptide sequence shown in italic; 4D5scFv sequence shown in double | |
| | underline; linker sequence shown in lowercase; GTB sequence shown in bold; | |
| | Myc sequence shown in italic double underline; his tag sequence shown in | |
| | bold italic.) | |
| 4D5scFv- | METDTLLLWVLLLWVPGSTGEVQLVESGGGLVQPGGSLRLSCAASGFNIKD | 62 |
| GTA-Myc-His | TYIHWVRQAPGKGLEWVARIYPTNGYTRYADSVKGRFTISADTSKNTAYLQ | |
| tag | MNSLRAEDTAVYYCSRWGGDGFYAMDYWGQGTLVTVSSGGGGSGGGGS | |
| | GGGGSDIQMTQSPSSLSASVGDRVTITCRASQDVNTAVAWYQQKPGKAPK | |
| | LLIYSASFLYSGVPSRFSGSRSGTDFTLTISSLQPEDFATYYCQQHYTTPPTF | |
| | GQGTKVEIKggggsggggsggggsEPDHLQRVSLPRMVYPQPKVLTPCRKDVLV | |
| | VTPWLAPIVWEGTFNIDILNEQFRLQNTTIGLTVFAIKKYVAFLKLFLETAEK | |
| | HFMVGHRVHYYVFTDQPAAVPRVTLGTGRQLSVLEVRAYKRWQDVSMRR | |
| | MEMISDFCERRFLSEVDYLVCVDVDMEFRDHVGVEILTPLFGTLHPGFYGS | |
| | SREAFTYERRPQSQAYIPKDEGDFYYLGGFFGGSVQEVQRLTRACHQAM | |
| | MVDQANGIEAVWHDESHLNKYLLRHKPTKVLSPEYLWDQQLLGWPAVLR | |
| | KLRFTAVPKNHQAVRNP [symbol] [symbol] | |
| | (Signal peptide sequence shown in italic; 4D5scFv sequence shown in double | |
| | underline; linker sequence shown in lowercase; GTA sequence shown in bold; | |
| | Myc sequence shown in italic double underline; his tag sequence shown in | |
| | bold italic.) | |

In some embodiments, the bi-functional therapeutic includes a first protein a first protein comprising the amino acid sequence of positions 20-774 of SEQ ID NO: 50 or SEQ ID NO: 51 and a second protein comprising the amino acid sequence of positions 20-233 of SEQ ID NO: 52.

In some embodiments, the bi-functional therapeutic includes a first protein comprising the amino acid sequence of positions 20-563 of SEQ ID NO: 53 or SEQ ID NO: 54 and a second protein comprising the amino acid sequence of positions 20-233 of SEQ ID NO: 55, optionally where the bi-functional therapeutic comprises a first portion comprising the amino acid sequence of positions 20-569 of SEQ ID NO: 53 or SEQ ID NO: 54.

In some embodiments, the bi-functional therapeutic includes a first protein comprising the amino acid sequence of SEQ ID NO: 56 or SEQ ID NO: 57 and a second protein comprising the amino acid sequence of positions 1-214 of SEQ ID NO: 58.

In some embodiments, the bi-functional therapeutic includes the amino acid sequence of positions 1-555 of SEQ ID NO: 59 or SEQ ID NO: 60.

In some embodiments, the bi-functional therapeutic includes the amino acid sequence of positions 20-593 of SEQ ID NO: 61 or SEQ ID NO: 62, optionally where the bi-functional therapeutic comprises the sequence of positions 20-599 of SEQ ID NO: 61 or SEQ ID NO: 62.

Another aspect of the present disclosure relates to a bi-functional therapeutic for treating cancer that includes a targeting component which targets CD19 and a glycosyltransferase which, when delivered to a tumor by said targeting component, enzymatically converts the tumor phenotype to that of an incompatible allograft or xenograft, said glycosyltransferase being coupled to said targeting component. This aspect of the present disclosure is useful in treating a subject with a need for elimination of B-cells or B-cell activity. In some embodiments, this aspect of the present disclosure is useful to treat a lymphoma (e.g., a B cell lymphoma), a B-cell leukemia, and/or autoimmune diseases.

In some embodiments, the targeting component comprises a heavy chain variable region, where said heavy chain variable region includes: a complementarity-determining region 1 (CDR-H1) comprising an amino acid sequence of SEQ ID NO: 12, or a modified amino acid sequence of SEQ ID NO: 12, said modified sequence having at least 80% sequence identity to SEQ ID NO: 12; a complementarity-determining region 2 (CDR-H2) comprising an amino acid sequence of SEQ ID NO: 15, or a modified amino acid sequence of SEQ ID NO: 15, said modified sequence having at least 80% sequence identity to SEQ ID NO: 15; and a complementarity-determining region 3 (CDR-H3) comprising an amino acid sequence of SEQ ID NO: 18, or a modified amino acid sequence of SEQ ID NO: 18, said modified sequence having at least 80% sequence identity to SEQ ID NO: 18. The sequences of the heavy chain CDR sequences are provided in Table 1 above.

In some embodiments, the targeting component comprises a heavy chain variable region including an amino acid sequence that is at least 80% identical to SEQ ID NO: 32 (Table 3 above).

The targeting component may further comprise a light chain variable region, where said light chain variable region includes: a complementarity-determining region 1 (CDR-L1) having an amino acid sequence of SEQ ID NO: 21, or a modified amino acid sequence of SEQ ID NO: 21, said modified sequence having at least 80% sequence identity to SEQ ID NO: 21; a complementarity-determining region 2 (CDR-L2) having an amino acid sequence of SEQ ID NO: 24, or a modified amino acid sequence of SEQ ID NO: 24, said modified sequence having at least 80% sequence identity to SEQ ID NO: 24; and a complementarity-determining region 3 (CDR-L3) having an amino acid sequence of SEQ ID NO: 27, or a modified amino acid sequence of SEQ ID NO: 27, said modified sequence having at least 80% sequence identity to SEQ ID NO: 27. The sequences of the light chain CDR sequences are provided in Table 2 above.

In some embodiments, the light chain variable region includes an amino acid sequence that is at least 80% identical to SEQ ID NO: 32 (Table 3 above).

In some embodiments, the targeting component comprises a heavy chain variable region including the CDR-H1 of SEQ ID NO: 12, the CDR-H2 of SEQ ID NO: 15, and the CDR-H3 of SEQ ID NO: 18, and a light chain variable region including the CDR-L1 of SEQ ID NO: 21, the CDR-L2 of SEQ ID NO: 24, and the CDR-L3 of SEQ ID NO: 27.

In some embodiments, the targeting component comprises a heavy chain variable region including an amino acid sequence that is at least 80% identical to SEQ ID NO: 32 and a light chain variable region including an amino acid sequence that is at least 80% identical to SEQ ID NO: 33 (Table 3).

In some embodiments, the targeting component further includes a signaling peptide, optionally where the signaling peptide has the sequence of amino acids 1-19 of SEQ ID NO: 63.

Suitable glycosyltransferases are described in detail infra.

In some embodiments, the glycosyltransferase is selected from the group consisting of glycosyltransferase A (Alpha 1-3-N-Acetylgalactosaminyltransferase) and glycosyltransferase B (alpha 1-3-galactosyltransferase).

In some embodiments, the glycosyltransferase is Marmoset α-1,3 galactosyltransferase (aa90-376) having the sequence of SEQ ID NO: 66.

In some embodiments, the bi-functional therapeutic includes the amino acid sequence of SEQ ID NO: 63 (Table 6).

TABLE 6
Obexelimab Bi-Functional Therapeutic Protein Sequences

| | | SEQ ID |
|---|---|---|
| Protein Sequence | Sequence | NO: |
| human IL2 signal | MGWSCIILFLVATATGVHSEVQLVESGGGLVKPGGSLKLSCAASG | 63 |
| peptide-Obexelimab- | YTFTSYVMHWVRQAPGKGLEWIGYINPYNDGTKYNEKFQGRVTIS | |
| scFv-Marmoset α-1,3 | SDKSISTAYMELSSLRSEDTAMYYCARGTYYYGTRVFDYWGQGT | |
| galactosyltransferase | LVTVSSggggsggggsggggsggggs [symbol] | |
| (aa90-376)-his tag | [symbol] | |
| | [symbol] g | |
| | gggsggggsggggsELRLWDWFNPKKRPEVMTVTQWKAPVVWEGT | |
| | YNKAILENYYAKQKITVGLTVFAIGRYIEHYLEEFVTSANRYFMVG | |
| | HKVIFYVMVDDVSKAPFIELGPLRSFKVFEVKPEKRWQDISMMRM | |
| | KTIGEHILAHIQHEVDFLFCMDVDQVFQDHFGVETLGQSVAQLQA | |
| | WWYKADPDDFTYERRKESAAYIPFGQGDFYYHAAIFGGTPIQVL | |
| | NITQECFKGILLDKKNDIEAEWHDESHLNKYFLLNKPSKILSPEYC | |
| | WDYHIGLPSDIKTVKLSWQTKEYNLVRKNVGGGS [symbol] | |
| | (human IL2 signal peptide sequence shown in italic; obexelimab | |
| | sequence shown in double underline; linker sequence shown in | |
| | lowercase; scFv shown in italic bold double underline; linker | |
| | sequence shown in lowercase; Marmoset α-1,3 | |
| | galactosyltransferase (aa90-376) shown in bold; his tag sequence | |
| | shown in bold italic.) | |

In some embodiments, the bi-functional therapeutic includes the amino acid sequence of positions 20-584 of SEQ ID NO: 63, the amino acid sequence of positions 20-578 of SEQ ID NO: 63, the amino acid sequence of positions 20-287 of SEQ ID NO: 63, the amino acid sequence of positions 20-272 of SEQ ID NO: 63, the amino acid sequence of positions 20-160 of SEQ ID NO: 63, or the amino acid sequence of positions 20-140 of SEQ ID NO: 63.

It will be appreciated that the exact dosage of the bi-functional therapeutic of the present disclosure is chosen by the individual physician in view of the patient to be treated. In general, dosage and administration are adjusted to provide an effective amount of the agent to the patient being treated. As used herein, the “effective amount” of a bi-functional therapeutic refers to the amount necessary to elicit the desired biological response. As will be appreciated by those of ordinary skill in this art, the effective amount of bi-functional therapeutic of the present disclosure may vary depending on such factors as the desired biological endpoint, the drug to be delivered, the target tissue, the route of administration, etc. For example, the effective amount of bi-functional therapeutic might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

An “effective amount” may also be a “a prophylactically effective amount,” which refers to an amount of the bi-functional therapeutic as described herein, which is effective, upon single- or multiple-dose administration to the subject, in preventing or delaying the occurrence of the onset or recurrence of a disorder, e.g., a cancer, or treating a symptom thereof.

In general, doses can range from about 25% to about 100% of the maximum tolerated dose (MTD) of the bi-functional therapeutic when given as a single agent. Based upon the composition, the dose can be delivered once, continuously, such as by continuous pump, or at periodic intervals. Dosage may be adjusted appropriately to achieve desired drug levels, locally, or systemically. In the event that the response in a subject is insufficient at such doses, even higher doses (or effective higher doses by a different, more localized delivery route) may be employed to the extent that patient tolerance permits. Continuous IV dosing over, for example, 24 hours or multiple doses per day also are contemplated to achieve appropriate systemic levels of compounds. By way of example, the dosage schedule can be varied, such that the bi-functional therapeutic is administered once, twice, three or more times per week for any number of weeks or the bi-functional therapeutic is administered more than once (e.g., two, three, four, five, six, seven, eight, nine, ten, eleven, twelve, thirteen, fourteen, fifteen, sixteen, seventeen, eighteen, nineteen, twenty, twenty-two or twenty-four times) with administration occurring once a week, once every two, three, four, five, six, seven, eight, nine or ten weeks. For example, a bi-functional therapeutic can be administered at least two, three or four times at a dosage level recited above with administration occurring one every four to eight weeks. If the subject does not demonstrate an adverse reaction to the bi-functional therapeutic and/or one or more symptom of the cancer improves or remains the same, an additional dose or doses can be given. In some embodiments, as the period between dosing increases, the amount of bi-functional therapeutic can be increased.

The biodistribution and pharmacokinetics of the bi-functional therapeutic may be different for different targeting components. By way of example, a large bi-functional therapeutic comprised of a full length, intact antibody will have a longer plasma and whole body half-life and tend to remain in the circulation. Such bi-functional therapeutics will also be more likely to be excreted via the liver and less likely to penetrate into normal tissues. Conversely, a small bi-functional therapeutic comprised of a targeting peptide or small molecule ligand, for example, will tend to have a shorter half-life, be excreted via the kidney/urinary tract and penetrate normal tissues and tumors more readily.

In practicing the methods of the present disclosure, the administering step is carried out to treat cancer in a subject. In one embodiment, a subject having cancer is selected prior to the administering step. Such administration can be carried out systemically or via direct or local administration to the tumor site. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intra-arterialy, intra-lesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of the bi-functional therapeutic will vary depending on the type of the bi-functional therapeutic (e.g., having an antibody targeting component or a peptide targeting component) and the disease to be treated.

The bi-functional therapeutic of the present disclosure may be orally administered, for example, with an inert diluent, or with an assimilable edible carrier, or it may be enclosed in hard or soft shell capsules, or it may be compressed into tablets, or they may be incorporated directly with the food of the diet. The bi-functional therapeutic of the present disclosure may also be administered in a time release manner incorporated within such devices as time-release capsules or nanotubes. Such devices afford flexibility relative to time and dosage. For oral therapeutic administration, the agents of the present disclosure may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of the bi-functional therapeutic of the present disclosure in such therapeutically useful compositions is such that a suitable dosage will be obtained.

When the bi-functional therapeutic of the present disclosure is administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the bi-functional therapeutic of the present disclosure systemically, it may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the bi-functional therapeutic of the present disclosure can also be achieved using infusion pump devices. Such devices allow continuous infusion of desired compounds avoiding multiple injections and multiple manipulations.

In addition to the formulations described previously, the bi-functional therapeutic may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

Another aspect of the present disclosure relates to a pharmaceutical composition comprising the bi-functional therapeutic of the present disclosure and a pharmaceutically acceptable carrier.

Bi-functional therapeutics are described above.

Pharmaceutical compositions containing the bi-functional therapeutic for use in the methods of the present disclosure can include a pharmaceutically acceptable carrier as described infra, one or more active agents, and a suitable delivery vehicle. Suitable delivery vehicles include, but are not limited to, viruses, bacteria, biodegradable microspheres, microparticles, nanoparticles, liposomes, collagen minipellets, and cochleates.

In one embodiment of the present disclosure, the pharmaceutical composition or formulation is encapsulated in a lipid formulation to form a nucleic acid-lipid particle as described in Semple et al., “Rational Design of Cationic Lipids for siRNA Delivery,” Nature Biotech. 28:172-176 (2010), WO2011/034798 to Bumcrot et al., WO2009/111658 to Bumcrot et al., and WO2010/105209 to Bumcrot et al., which are hereby incorporated by reference in their entirety.

In another embodiment of the present disclosure, the delivery vehicle is a nanoparticle. A variety of nanoparticle delivery vehicles are known in the art and are suitable for delivery of the bi-functional therapeutic of the present disclosure (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety), and liposome-entrapped siRNA nanoparticles (Kenny et al., “Novel Multifunctional Nanoparticle Mediates siRNA Tumor Delivery, Visualization and Therapeutic Tumor Reduction In Vivo,” J. Control Release 149(2): 111-116 (2011), which is hereby incorporated by reference in its entirety). Other nanoparticle delivery vehicles suitable for use in the present disclosure include microcapsule nanotube devices disclosed in U.S. Patent Publication No. 2010/0215724 to Prakash et al., which is hereby incorporated by reference in its entirety.

In another embodiment of the present disclosure, the pharmaceutical composition is contained in a liposome delivery vehicle. The term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Several advantages of liposomes include: their biocompatibility and biodegradability, incorporation of a wide range of water and lipid soluble drugs; and they afford protection to encapsulated drugs from metabolism and degradation. Important considerations in the preparation of liposome formulations are the lipid surface charge, vesicle size, and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Methods for preparing liposomes for use in the present disclosure include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda, and U.S. Pat. No. 5,059,421 to Loughrey et al., which are hereby incorporated by reference in their entirety.

In another embodiment of the present disclosure, the delivery vehicle is a viral vector. Viral vectors are particularly suitable for the delivery of nucleic acid molecules, but can also be used to deliver molecules encoding the bi-functional therapeutic. Suitable gene therapy vectors include, without limitation, adenoviral vectors, adeno-associated viral vectors, retroviral vectors, lentiviral vectors, and herpes viral vectors.

Adenoviral viral vector delivery vehicles can be readily prepared and utilized as described in Berkner, “Development of Adenovirus Vectors for the Expression of Heterologous Genes,” Biotechniques 6:616-627 (1988), Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety. Adeno-associated viral delivery vehicles can be constructed and used to deliver the bi-functional therapeutic of the present disclosure to cells as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable in Vivo Expression of the Cystic Fibrosis Transmembrane Conductance Regulator With an Adeno-Associated Virus Vector,” Proc. Nat'l. Acad. Sci. 90:10613-10617 (1993) and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver a nucleic acid molecule to a target cell. One such type of retroviral vector is disclosed in U.S. Pat. No. 5,849,586 to Kriegler et al., which is hereby incorporated by reference. Other nucleic acid delivery vehicles suitable for use in the present disclosure include those disclosed in U.S. Patent Publication No. 20070219118 to Lu et al., which is hereby incorporated by reference in its entirety.

Regardless of the type of infective transformation system employed, it should be targeted for delivery of the nucleic acid to the desired cell type. For example, for delivery into a cluster of cells (e.g., cancer cells) a high titer of the infective transformation system can be injected directly within the site of those cells so as to enhance the likelihood of cell infection. The infected cells will then express the nucleic acid molecule targeting the tumor-associated antigen. The expression system can further contain a promoter to control or regulate the strength and specificity of expression of the nucleic acid molecule in the target tissue or cell.

As described supra, effective doses of the compositions of the present disclosure, for the treatment of a metastatic disease vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

The pharmaceutical compositions of the present disclosure may include a “therapeutically effective amount” or a “prophylactically effective amount” of a bi-functional therapeutic of the present disclosure. A “therapeutically effective amount” refers to an amount effective, at dosages and for periods of time necessary, to achieve the desired therapeutic result. A therapeutically effective amount of the bi-functional therapeutic may vary according to factors such as the disease state, age, sex, and weight of the individual, and the ability of the bi-functional therapeutic to elicit a desired response in the individual. A therapeutically effective amount is also one in which any toxic or detrimental effects of the bi-functional therapeutic is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In certain embodiments, the administering step further comprises administering the nucleotide sugar uridine diphosphate galactose (UDP-gal), uridine diphosphate-N-acetylgalactosamine (UDP-NAcGal), and/or guanosine diphosphate-fucose (GDP-fucose).

The UDP-gal, UDP-NAcGal, and/or GDP-fucose may be administered by any suitable route, including but not limited to intravenous, subcutaneous, intramuscular, intraperitoneal, oral, rectal, or any other route known in the art. In addition, the UDP-gal, UDP-NAcGal, and/or GDP-fucose may be administered concurrent with or subsequent to the bi-functional targeted enzyme. In the latter case, i.e., subsequent administration, the interval between the targeted enzyme and the nucleotide sugar may range from 1 minute to 1 week. In a preferred embodiment, the interval ranges from 1 minute to 48 hours.

The bi-functional therapeutic described herein may be used in combination with other therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is referred to herein as “simultaneous” or “concurrent delivery.” In other embodiments, the delivery of one treatment ends before the delivery of the other treatment begins. In some embodiments, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Exemplary therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020, which is hereby incorporated by reference in its entirety), CC-1065 (see U.S. Pat. Nos. 5,475,092, 5,585,499, 5,846,545, which are hereby incorporated by reference in their entirety) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechloretharnine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In other embodiments, the bi-functional therapeutic is administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

In other embodiments, the bi-functional therapeutic is administered in combination with an immunomodulatory agent, e.g., IL-1, IL-24, IL-6, or IL-12, or interferon alpha or gamma.

A further aspect of the present disclosure provides a nucleic acid (for example a polynucleotide) molecule encoding the bi-functional therapeutic of the present disclosure. The polynucleotide may be, for example, DNA, cDNA, PNA, RNA or combinations thereof, either single- and/or double-stranded, or native or stabilized forms of polynucleotides, such as, for example, polynucleotides with a phosphorothioate backbone and it may or may not contain introns so long as it codes for the bi-functional therapeutic. Of course, only peptides that contain naturally occurring amino acid residues joined by naturally occurring peptide bonds are encodable by a polynucleotide. A still further aspect of the present disclosure provides a recombinant expression vector capable of expressing a bi-functional therapeutic according to the present disclosure. A variety of methods have been developed to link polynucleotides, especially DNA, to vectors for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. Synthetic linkers containing a variety of restriction endonuclease sites are commercially available from a number of sources including International Biotechnologies Inc. New Haven, CN, USA. A desirable method of modifying the DNA encoding the bi-functional therapeutic of the present disclosure employs the polymerase chain reaction as disclosed by Higuchi et al., “A General Method of In Vitro Preparation and Specific Mutagenesis of DNA Fragments: Study of Protein and DNA Interactions,” Nucleic Acids Res. 16(15):7351-67 (1988), which is hereby incorporated by reference in its entirety. This method may be used for introducing the DNA into a suitable vector, for example by engineering in suitable restriction sites, or it may be used to modify the DNA in other useful ways as is known in the art.

The nucleic acids of the present disclosure may be chosen for having codons, which are preferred, or non-preferred, for a particular expression system. By way of example, the nucleic acid can be one in which at least one codon, preferably at least 10% or 20% of the codons, has been altered such that the sequence is optimized for expression in E. coli., yeast, human, insect, NS0, or CHO cells.

Typically, the polynucleotide that encodes the bi-functional therapeutic is placed under the control of a promoter that is functional in the desired host cell. A wide variety of promoters are well known, and can be used in the expression vectors of the present disclosure, depending on the particular disclosure. Ordinarily, the promoter selected depends upon the cell in which the promoter is to be active. Other expression control sequences such as ribosome binding sites, transcription termination sites and the like are also optionally included. Constructs that include one or more of these control sequences are termed “expression vectors.” Accordingly, the present disclosure provides expression vectors into which the nucleic acid molecules that encode bi-functional therapeutics are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature 198:1056 (1977), which is hereby incorporated by reference in its entirety), the tryptophan (trp) promoter system (Goeddel et al., Nucleic Acids Res. 8:4057 (1980), which is hereby incorporated by reference in its entirety), the tac promoter (DeBoer, et al., Proc. Natl. Acad. Sci. U.S.A. 80:21-25 (1983), which is hereby incorporated by reference in its entirety); and the lambda-derived P_{L} promoter and N-gene ribosome binding site (Shimatake et al., Nature 292:128 (1981), which is hereby incorporated by reference in its entirety). However, any available promoter that functions in prokaryotes can be used.

For expression of the bi-functional therapeutic in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in the expression cassettes of the present disclosure. An RBS in E. coli, for example, consists of a nucleotide sequence 3-9 nucleotides in length located 3-11 nucleotides upstream of the initiation codon (Shine and Dalgarno, “Determinant of Cistron Specificity in Bacterial Ribosomes,” Nature 254:34-38 (1975); Steitz, In Biological regulation and development: Gene expression (ed. R. F. Goldberger), vol. 1, p. 349, 1979, Plenum Publishing, New York), which are hereby incorporated by reference in their entirety).

For mammalian cells, the control sequences will include a promoter and preferably an enhancer derived from immunoglobulin genes, SV40, cytomegalovirus, etc., and a polyadenylation sequence, and may include splice donor and acceptor sequences.

Either constitutive or regulated promoters can be used in the present disclosure. Regulated promoters can be advantageous because the host cells can be grown to high densities before expression of the bi-functional therapeutic is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the bi-functional therapeutic. For E. coli and other bacterial host cells, inducible promoters are known to those of skill in the art. These include, for example, the lac promoter, the bacteriophage lambda P_{L} promoter, the hybrid trp-lac promoter (Amann et al. Gene 25:167 (1983); de Boer et al. Proc. Nat'l. Acad. Sci. USA 80:21 (1983), which are hereby incorporated by reference in their entirety), and the bacteriophage T7 promoter (Studier et al. J. Mol. Biol (1986).; Tabor et al. Proc. Nat'l. Acad. Sci. USA 82: 1074-8 (1985), which are hereby incorporated by reference in their entirety).

Selectable markers are often incorporated into the expression vectors used to express the bi-functional therapeutic of the present disclosure. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art.

Construction of suitable nucleic acid constructs containing one or more of the above listed components employs standard ligation techniques as described in the references cited above. Isolated plasmids or DNA fragments are cleaved, tailored, and re-ligated in the form desired to generate the nucleic acid constructs (e.g., plasmids) required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel), which are hereby incorporated by reference in their entirety.

A variety of common vectors suitable for use as starting materials for constructing the nucleic acid constructs and expression vectors of the present disclosure are well known in the art. For cloning in bacteria, common vectors include pBR322 derived vectors such as pBLUESCRIP™, and λ-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g. vaccinia virus, adeno virus, and baculovirus), episomal virus vectors (e.g., bovine papillomavirus), and retroviral vectors (e.g., murine retroviruses).

The nucleic acid may then be expressed in a suitable host to produce a polypeptide comprising the bi-functional therapeutic of the present disclosure. Thus, the nucleic acid encoding the bi-functional therapeutic of the present disclosure may be used in accordance with known techniques, appropriately modified in view of the teachings contained herein, to construct an expression vector, which is then used to transform an appropriate host cell for the expression and production of the bi-functional therapeutic of the present disclosure. Such techniques are described infra and also include those disclosed, for example, in U.S. Pat. Nos. 4,440,859, 4,530,901, 4,582,800, 4,677,063, 4,678,751, 4,704,362, 4,710,463, 4,757,006, 4,766,075, and 4,810,648, which are hereby incorporated by reference in their entirety.

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

The bi-functional therapeutics of the present disclosure can also be further linked to other bacterial proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series. For certain applications, it may be desirable to cleave the non-enzyme amino acids from the fusion protein after purification. This can be accomplished by any of several methods known in the art, including cleavage by cyanogen bromide, a protease, or by Factor Xa (see, e.g., Itakura et al., Science (1977) 198: 1056; Goeddel et al., Proc. Natl. Acad. Sci. USA (1979) 76: 106; Nagai et al., Nature (1984) 309: 810; Sung et al., Proc. Natl. Acad. Sci. USA (1986) 83: 561, which are hereby incorporated by reference in their entirety). Cleavage sites can be engineered into the gene for the fusion protein at the desired point of cleavage.

More than one bi-functional therapeutic may be expressed in a single host cell by placing multiple transcriptional cassettes in a single expression vector, or by utilizing different selectable markers for each of the expression vectors which are employed in the cloning strategy.

The bi-functional therapeutics can be purified according to standard procedures of the art, including ammonium sulfate precipitation, affinity columns, column chromatography, gel electrophoresis and the like (see, generally, R. Scopes, Protein Purification, Springer-Verlag, New York (1982), Deutscher, Methods in Enzymology Vol. 182: Guide to Protein Purification., Academic Press, Inc. New York (1990), which is hereby incorporated by reference in its entirety). Substantially pure compositions of at least about 70 to 90% homogeneity are preferred, and 98 to 99% or more homogeneity are most preferred. By way of example, when the targeting component of the bi-functional therapeutic is an antibody, antibody binding chromatography, such as ion exchange chromatography, can be used. The ion exchange chromatography can be anion exchange chromatography, cation exchange chromatography, or both. Types of anion exchange chromatography include, without limitation, Q Sepharose Fast Flow®, MacroPrep High Q Support®, DEAE Sepharose Fast Flow®, and Macro-Prep DEAE®. Types of cation exchange chromatography include, without limitation, SP Sepharose Fast Flow®, Source 30S®, CM Sepharose Fast Flow®, Macro-Prep CM Support®, and Macro-Prep High S Support®.

To facilitate purification of the bi-functional therapeutics of the present disclosure, the nucleic acids that encode the bi-functional therapeutics can also include a coding sequence for an epitope or “tag” for which an affinity binding reagent is available, i.e. a purification tag. Examples of suitable epitopes include the myc and V-5 reporter genes; expression vectors useful for recombinant production of fusion proteins having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the bi-functional therapeutic of the present disclosure, and corresponding detection systems are known to those of skill in the art, and several are commercially available (e.g., “FLAG” (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, New York, which is hereby incorporated by reference in its entirety; commercially available from Qiagen (Santa Clarita, Calif.)).

Purification tags also include maltose binding domains and starch binding domains. Purification of maltose binding domain proteins is known to those of skill in the art. Starch binding domains are described in WO 99/15636, which is hereby incorporated by reference in its entirety.

### EXAMPLES

The examples below are intended to exemplify the practice of embodiments of the disclosure but are by no means intended to limit the scope thereof.

### Materials and Methods

Cell lines. Human prostate cancer cell lines LNCaP and PC3 were purchased from American Type Culture Collection (Manassas, VA). CWR22Rv1 was a gift from Thomas Pretlow, MD, Case Western Reserve University. Breast cancer cell line MDA-MB-361 was a gift from Christel Larbouret, (Institute of Cancer Research of Montpellier (France)). LNCaP, PC3 and CWR22Rv1 were maintained in RPMI1640 medium supplemented with 2 mM L-glutamine, 1% penicillin-streptomycin and 10% heat inactivated fetal bovine serum (FBS) (all supplements from Gemini Bio-products, West Sacramento, CA). MDA-MB-361 was maintained in L-15 medium (ATCC) supplemented with 1% penicillin-streptomycin and 20% FBS.

Antibodies. Monoclonal antibody (mAb) J591 anti-FOLH1/PSMA, murine and de-immunized, were generated as described in Liu et al., “Monoclonal Antibodies to the Extracellular Domain of Prostate Specific Membrane Antigen Also React With Tumor Vascular Endothelium,” Cancer Res. 57:3629-3634 (1997), U.S. Pat. No. 7,045,605 to Bander et al., and U.S. Pat. No. 7,514,078 to Bander et al., which are hereby incorporated by reference in their entirety. MAb 3E6 anti-PSMA, horseradish peroxidase-labeled polymer conjugated goat anti-mouse Ig and horseradish peroxidase conjugated rabbit anti-human IgG were purchased from Dako (Carpinteria, CA). MAb 4D5 was purchased as Herceptin (Genentech/Roche). MAb anti-A and anti-B antibodies were purchased from Ortho Diagnostic Systems (Raritan, NJ). Ulex europaeus lectin that recognizes alpha-linked fucose residues for detection of the O/H antigen was purchased from Sigma-Aldrich (St. Louis, MO). Donkey anti-human IgG, horseradish peroxidase-conjugated donkey anti-human IgG, alkaline phosphatase-conjugated donkey anti-human IgG, FITC-conjugated donkey anti-mouse Ig and FITC-conjugated donkey anti-human Ig were purchased from Jackson ImmunoResearch Laboratories (West Grove, PA). Anti-Flag M2 was from Sigma-Aldrich. IRDye 800CW-goat anti-mouse secondary antibody was purchased from LI-COR Biosciences (Lincoln, Nebraska).

DNA plasmids used in this study. A series of plasmids can be constructed for any desired targeting Ab or Ab construct or peptide plus any glycosyltransferase including but not limited to GTB, GTA and fucosyltransferase (FUT1 or FUT2) as outlined in the example below.

DNA plasmids and their protein products over-expressed in host cells after transfection or co-transfection are listed below with brief descriptions. Each plasmid is described as: Designation of DNA plasmid (its protein product): brief description. pMG 145 (H chain): transfection of this plasmid into host cells generates huJ591 heavy chain. pMG 135 (L chain): transfection of this plasmid generates huJ591 light chain (L). pMG 145 andpMG 135 (huJ591 antibody): co-transfection of these two plasmids results in co-expression of heavy and light chains and functional huJ591 antibody. pMG 181 (H chain-GTB): transfection of this plasmid generates a fusion protein with huJ591 heavy chain (H) at the N-terminus and GTB at C-terminus (see below). pMG 181 and pMG 135 (huJ591-GTB fusion antibody): co-transfection of these two plasmids produces heavy and light chains including GTB.

Construction of GTB and huJ591 or 4D5 heavy chain-GTB (H-GTB) fusion expression plasmids. The region of alpha 1,3 galactosyltransferease (GTB) that includes the catalytic domain (amino acids 57-354) was subcloned by PCR using the GTB-encoding plasmid pBBBB as template. Flag and His tags may be added at the 3′ terminus, if desired, to follow or aid in the purification of the fusion proteins. For example, to construct the antibody-GTB fusion protein, DNA sequence encoding huJ591 heavy chain (H) was ligated to the GTB catalytic domain DNA sequence, resulting in plasmid pMG181. The same procedure is also followed to generate 4D5-GTB or any Ab (or Ab derivative or peptide)-GTB (or the catalytic domain of GTA for the A antigen). Or, in the case of desiring to target the synthesis of the H antigen, the catalytic domain of FUT1 or FUT2 can be incorporated. The glycosyltransferase enzymes are preferentially ligated at the C terminus of either the Heavy or Light chain of the Ab construct.

Between the Ab sequence and enzyme sequence, a (G₄S)₃ spacer sequence was inserted. Alternatively, various fusion protein linkers or spacers can be used as described by Chen et al., “Fusion Protein Linkers: Property, Design and Functionality,” Adv. Drug Deliv. Rev. 65(10):1357-69 (2013), which is hereby incorporated by reference in its entirety.

DNA transfection and fusion protein expression. For huJ591-GTB (and analogously for 4D5-GTB) fusion antibody production, CHO cells were co-transfected with pMG181 (H-chain-GTB) and pMG135 (L-chain) using FreeStyle MAX (ThermoFisher scientific) following the manufacturer's instructions. Supernatants containing the fusion antibody were harvested 5 days after transfection and concentrated by Amicon Ultra 10K centrifugal filter (Merck Millipore).

Purification of over-expressed fusion protein. huJ591 was purified using protein G-sepharose (GE healthcare) following the manufacturer's instructions. J591-GTB was purified using ANTI-FLAG M2 affinity gel (Sigma-Aldrich) following the manufacturer's instructions. In brief, supernatant containing the fusion protein was incubated with M2 affinity gel for 2 hours followed by washing, eluting with 3×FLAG peptide (Sigma-Aldrich), and dialysis against PBS.

Western blot analyses. Supernatant containing fusion protein or purified fraction was separated by a 4-20% SDS-PAGE gel (Life Technology) under reducing and non-reducing conditions, and transferred to a polyvinylidene difluoride membrane (PVDF) (Millipore, Billerica, MA). The membrane was blocked with 5% dry milk/PBST for 60 minutes. Anti-flag M2 was incubated with the membrane for 60 minutes. After washing, TRDye 800CW-goat anti-mouse secondary antibody was incubated with the membrane for 60 minutes. After washing, the membrane was analyzed using Odyssey Infrared Imaging System (LI-COR Biosciences).

Immunostaining. Cells (2×10⁵/well) were grown on cover slips in 12-well plates for 24 hours prior to experiments. Cells were fixed with 4% paraformaldehyde (PFA) in PBS, followed by 3 PBS washes. For detection of human histo-blood group antigens, murine monoclonal anti-A or anti-B antibody was added for 60 minutes at RT. After washing with PBS, cells were incubated with FITC-conjugated donkey anti-mouse immunoglobulin for 60 minutes and washed with PBS. Expression of HBGA O was detected by incubating cells with FITC-conjugated Ulex europaeus agglutinin for 60 minutes at RT followed by visualization under an UV microscope. For detection of PSMA, huJ591 was added for 60 minutes at RT. After PBS washes, cells were stained with FITC-conjugated anti-human Ig for 60 minutes and washed with PBS. Cover slips were mounted and examined under an UV microscope.

For immunostaining of tissue sections from xenograft tumors, 3E6 was used for detection of PSMA expression in paraffin sections, and huJ591 for frozen sections. Antibodies for blood group antigen were the same as above. The paraffin sections were deparaffinized by placing slides in Histo-Clear followed by rehydrating through graded alcohols and washing in Tris-buffered saline-Tween 20 (TBST). The deparaffinized and rehydrated sections were placed in Target Retrieval Solution pH 9.0 (Dako) and heated in a water bath (95-99° C.) for 30 minutes. The sections were washed in TBST. Peroxidase block was added for 5 minutes. After washing in TBST, the mAbs were added for 60 minutes at RT. Antibody binding was detected using peroxidase-labeled polymer conjugated goat anti-mouse Ig and 3,3′-diaminobenzidine (DAB) substrate. The sections were visualized after counterstaining with 10% hematoxylin. The frozen sections were used for detection of J591-GTB fusion antibody bound to cell surface PSMA in vivo. The frozen sections were fixed with pre-cooled acetone for 10 minutes then washed in PBS. Peroxidase block was added for 5 minutes. After washing in PBS, J591-GTB was detected with a horseradish peroxidase conjugated rabbit anti-human IgG followed by DAB and counterstaining as described above. Sections incubated directly with huJ591 were used as a positive control.

Competition ELISA. Plates were coated overnight at 4′C with 7E11 antibody (an antibody that binds the N-terminus/cytoplasmic domain of PSMA) at 15 μg/ml in 0.05 M carbonate buffer (pH 9.5). The wells were blocked with 2% HSA in PBS for 30 minutes at RT and washed. LNCaP cell lysate (containing PSMA) at 1:8 dilution was added for 60 minutes at RT. After washing with PBS, serial dilutions of murine J591 antibody (30 μl) were added for 60 minutes and then co-incubated with supernatants containing J591-GTB or huJ591 (1.6 μg Ig/ml; 30 μl) at 4° C. overnight. After washing, donkey anti-human IgG-alkaline phosphatase (1:1,000) was added for 60 minutes at RT. After washing, the plates were incubated with pNPP (Sigma) and read at 405 nm.

Blood group antigen conversion in vitro. Cells (2×10⁵) were grown on cover slips in 12-well plates for 24 hours. The cover slips were washed with PBS and transferred to a wet chamber. The cells were then incubated with huJ591-GTB (or 4D5-GTB) fusion antibody plus UDP-galactose for 30 minutes at 37° C. After washing with PBS, the cells were fixed with PFA. B antigen conversion on cell surface was detected by immunostaining as described above.

Lytic activity of normal human O or A serum after cancer cells conversion to HBGA B in vitro. LNCaP cells were grown on 60 well microtiter plates. Cells were incubated with either native J591 or J591-GTB fusion protein or neither agent; all wells also got UDP-gal. Subsequently, sera from type A or O patients were added as a source of natural anti-B Ab and complement; control wells got J591 without GTB or no serum. After 3 hours, wells were washed, fixed with methanol and incubated with 2% Giemsa stain for 25 minutes before washing and reading. A similar method was used to test a larger panel of prostate and breast cancer cell lines in suspension. Lytic activity was evaluated both by trypan blue exclusion and by propidium iodide uptake measured by FACS.

Blood group antigen conversion in vivo. Under an Institutional Animal Care and Use Committee (IUCUC)-approved protocol, NOD SCID mice (Charles River, Wilmington, MA) aged 6-8 weeks were injected subcutaneously with 5×10⁶ cells suspended in 200 μl Matrigel (Corning Life Sciences, Bedford, MD). Cell lines LNCaP, CWR22Rv1, PC3 and MDA-MB-361 were used in animal experiments. After 14 to 21 days, established tumors reached 8 to 10 mm diameter. HuJ591, huJ591-GTB, 4D5, or 4D5-GTB was injected either intravenously (IV) or intratumorally. UDP-gal was injected either IV, intraperitoneally (IP), or subcutaneously (SQ). Mice were euthanized on days 1, 2, or 3, and tumors and other organs were harvested. Half of each tumor was prepared for frozen sections with OCT compound; the other half was placed in phosphate-buffered formalin for preparation of paraffin sections. Immunostaining is described above.

An intra-peritoneal xenograft model in NOD/SCID mice was also developed using the castrate-resistant human PC cell line C4-2-luciferase. In this model, human plasma can be injected IP to provide the natural Abs and complement without causing fluid overload when using the IV route. Several days after IP injection of 10×10⁶ C4-2-luc cells and after confirming tumor take by bio-luminescence imaging, 2 groups of 5 animals, each with comparable median/range of bio-luminescent photon flux, received a single IP treatment with J591-GTB, UDP-gal and human type O serum. For the control group, the type O serum was heat-inactivated prior to injection. The total flux of each animal was measured every 3-4 days for approximately 2 weeks.

### Example 1—Generating Antibody-Glycosyltransferase Fusion Proteins

First, a chimeric protein was generated that was composed of tumor targeted Ab and glycosyltransferase, a prototypic construct that provides a highly versatile, modular system possessing multiple functionalities: (1) the Ab specificity is interchangeable to allow targeting of different tumor-associated antigens. Examples of such tumor antigen targets include, but are not limited to: FOLH1/PSMA, VEGFr, CD19, CD20, CD25, CD30, CD33, CD38, CD52, CD79, B-Cell Maturation Antigen (BCMA), Somatostatin receptor (e.g., SSTR1-5), 5T4, gp100, CEA, mammoglobin A, melan A/MART-1, PSA, tyrosinase, HER-2/neu, EGFr, hTERT, MUC1, mesothelin, Nectin-4, TROP-2, and many others known in the art. The targeting portion of the structure can vary from intact (full length dimeric) to monomeric single chain Ab structures, Fab, Fab′2, scFv or other Ab fragment derivatives such as minibodies, diabodies, triabodies, etc. They may maintain or delete the FcRn-binding domain. Alternatively, the targeting moiety can be a peptide that binds to the targeted antigen; examples include but are not limited to a glutamate-urea-lysine derivative such as ACUPA (2-(3-((S)-5-amino-1-carboxypentyl)ureido) pentanedioic acid) that binds FOLH1/PSMA, a somatostatin derivative that binds SSTR2, Arg-Gly-Asp (RGD) peptide that binds alpha-v/beta-3 integrin that is expressed on proliferating endothelial cells and other targeting peptides known in the art. These varieties of targeting agents and their differing physical properties allow tailoring of different pharmacokinetics and biodistributions. For example, larger molecular constructs such as full length, intact Ab including the FcRn (neonatal receptor) binding site will have longer plasma and whole body half-lives and tend to remain in the circulation; they will more likely be excreted via the liver rather than kidney; less likely penetrate into normal tissues due to intervening normal cell layers and tight junctions. Conversely, constructs that are smaller, lack FcRn binding, made with a targeting peptide rather than antibody, will tend to have shorter half-lives, more likely be excreted via the kidney/urinary tract and penetrate normal tissues and tumors more readily. In addition to the specificity of target binding, these differing physical properties, PK and bio-distributions will influence the adverse event profile of the constructed agent. (2) the glycosyltransferase component can be varied based on the substantial body of knowledge of naturally occurring allelic variants and their respective properties that can be exploited to tailor its functionality. It may also include the alpha-gal-transferase that generates the highly immunogenic alpha-gal epitope that is naturally absent in humans. Use of any enzyme involved in post-translational modification is possible. In addition to glycosylation, other examples are phosphorylation and lipidation.

As an additional alternative to the generation of a genetically engineered fusion protein, one can accomplish the linkage of targeting agent and post-translational enzyme by use of chemical linkage of the 2 individual moieties. Such chemical linkages are known to those in the art.

For initial proof of concept efforts, 3 well-characterized, clinically validated Abs were selected: J591 (anti-FOLH1/PSMA (folate hydrolase-1/prostate-specific membrane antigen)), 4D5 (trastuzumab; anti-her2), and obexelimab (anti-CD19); 4 Ab structures: intact dimeric, intact monomeric, Fab and scFv, and four glycosyltransferase variants: α 1,3 galactosyltransferase (GTB; AF134414), alpha 1-3-N-acetylgalactosaminyltransferase (GTA; AF134415), α-1,3-galactosyltransferase (α-1,3-GalT or α-GalT; EC 2.4.1.87), and a completely novel structure described below. GTB transfers a galactose moiety from the nucleotide-donor UDP-gal in an α1,3 linkage to the acceptor H antigen to form Gal α (1-3)[Fuc α (1-2)]Gal β1,4 GlcNAc-R (HBGA B); GTB requires the α1-2-linked fucose modification of the H antigen for activity because the B transferase does not add to an unmodified type-2 precursor. α-1,3-GalT transfers a galactose moiety from the nucleotide-donor UDP-gal in an α1,3 linkage to Gal β1,4 GlcNAc-R; this enzyme does not require the α1-2-linked fucose modification of the H antigen for activity. GTB was selected because HBGA type O and A individuals constitute 85-90% of the population (Galili et al., “A Unique Natural Human IgG Antibody With Anti-Alpha-Galactosyl Specificity,” J Exp. Med. 160:1519-1531 (1984), which is hereby incorporated by reference in its entirety) and, as noted previously, these individuals harbor high levels of anti-HBGA B antibodies. α-1,3-GalT was chosen because it can add the terminal Gal to cells that do not form the H-antigen such as those derived from hematopoietic or mesenchymal cells. The choice of GTB benefits further as a result of the high level of polyclonal anti-gal activity (responsible for hyper-acute rejection of xenografts) that cross-reacts with HBGA B (Macher et al., “The Gal alpha1,3Gal beta1,4GlcNAc-R (alpha-Gal) Epitope: a Carbohydrate of Unique Evolution and Clinical Relevance,” Biochim. Biophys. 1780:75-88 (2008), which is hereby incorporated by reference in its entirety) as a result of their substantially identical structures. From the GTB (or GTA, α-GalT, or FUT) sequence, the short cytoplasmic, trans-membrane and stem regions that are not necessary for enzymatic activity were excised and replaced with the respective antibody (or derivative) or peptide sequence creating a chimeric protein whose membrane binding becomes reconstituted via the antibody or peptide domain binding its cognate antigen located on the plasma membrane. ELISA assays of the chimeric protein confirmed the respective Ab binding specificity and immunoreactivity remained intact (FIGS. 2A-2B) irrespective of whether intact or antibody fragment was used. Incorporation of 14C-gal from UDP-14C-gal into a synthetic substrate (fucosyl-lactose (FL)) was also measured as described by Yamamoto et al., “Amino Acid Residue at Codon 268 Determines Both Activity and Nucleotide-Sugar Donor Substrate Specificity of Human Histo-Blood Group A and B Transferases. In Vitro Mutagenesis Study,” J Biol. Chem. 271:10515-10520 (1996), which is hereby incorporated by reference in its entirety, (FIGS. 2A-2B) that confirmed maintenance of high GTB activity.

In addition to creating such fusion proteins by genetic engineering, one of knowledge in the art can chemically link a targeting protein, peptide or other biologic to an effector enzyme (e.g., glycosyltransferases) that can post-translationally modify cellular proteins.

### Example 2—Tailoring the Functionality of Glycosyltransferase Activity

The deep knowledge regarding the A, B and O alleles provides ample opportunities to further refine the functionality of this component. For example, among alternative allelic variants that could be selected is the so-called “cis A,B” sequence in which the 2 most critical amino acid residues (aa 266 and 268 of GTA (leu and gly) and GTB (meth and ala) are interchanged to generate a hybrid sequence (meth and gly) (Yazer et al., “The Cis-AB Blood Group Phenotype: Fundamental Lessons in Glycobiology,” Transfus. Med. Rev. 20:207-217 (2006), which is hereby incorporated by reference in its entirety). This cis A,B enzyme sequence synthesizes both HBGA A and B specificities.

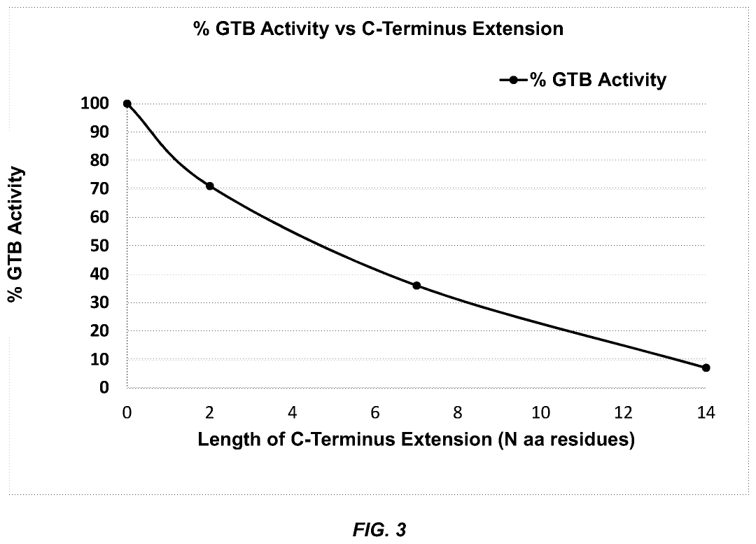

Other sequences are known which modulate the activity of the enzyme allowing one to titrate its potency. For example, a completely novel version of GTB was developed based on two naturally occurring mutant alleles of GTA (designated Ae101 and A201). Ae101 has a single base insertion and A201 has a single base deletion; each result in a frameshift. The frameshifts produce transferases with 37 and 21 amino acid extensions, respectively, at their C-termini. These resulting transferases, with their extensions, have enzymatic activity that is reduced by 30-50 fold or more (Yip, “Sequence Variation at the Human ABO Locus,” Annals of Human Genetics 66:1-27 (2002), which is hereby incorporated by reference in its entirety). While these 2 mutant alleles were defined in the context of GTA, no such mutant alleles have been described in the case of GTB. Nevertheless, completely novel sequences were generated by directly incorporating C-terminal sequence extensions into GTB by inserting a variety of amino acid sequences of varied length prior to the termination codon. When 4 versions of GTB that incorporated extensions of 2, 7, 14 and 54 amino acids were tested, the GTB activity was reduced progressively by up to 93% (FIG. 3) providing a mechanism whereby one can dial in the desired level of activity as well as an off-on switch as described above by incorporating a cleavable sequence that would jettison the extension in the presence of tumor- or tissue-related endoproteases or endopeptidases such as PSA, metalloproteinases, etc. The optional, sequence selection for the extension is at the option of the practitioner, its' only requirements being that it be selected to achieve the desired level of enzymatic activity, which can be measured as described below, and that it be non-immunogenic. Non-immunogenicity can be achieved by using sequence information of native, non-immunogenic proteins (e.g., albumin) or it can be achieved by methods known in the art to derive or determine immunogenicity for example by eliminating T-cell binding motifs.

### Example 3—Induced HBGA B Alloantigen Expression In Vitro

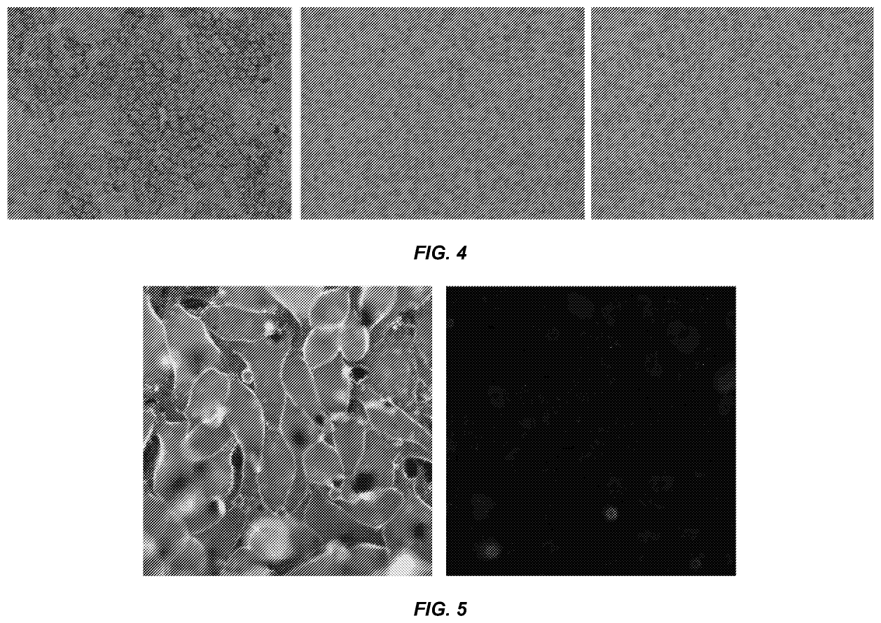

To demonstrate the functionality of the constructs, human prostate cancer cell lines LNCaP (PSMA-high), CWR22Rv1 (PSMA-heterogeneous and low), and PC-3 (PSMA-neg), all of which are naturally HBGA O, were incubated with chimeric J591 (anti-FOLH1/PSMA)-GTB or J591 (no GTB), both with UDP-gal, in vitro and on SCID mouse-derived xenograft tissue sections. Cell lines and tissue sections incubated with chimeric J591-GTB+UDP-gal converted to HBGA B whereas those incubated with J591 (without GTB)+UDP-gal did not, demonstrating that GTB was necessary for the conversion (FIG. 4).

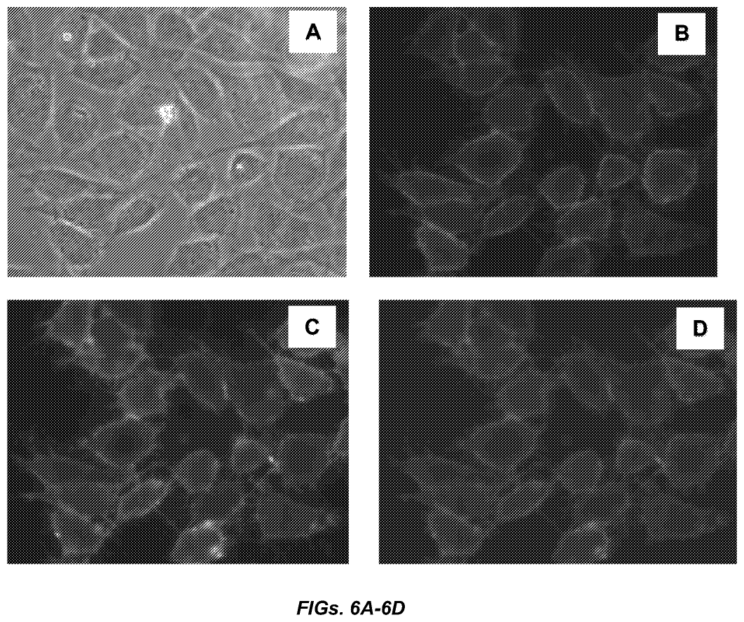

In vitro, while J591-GTB converted LNCaP (PSMA-high) from HBGA O to HBGA B, PC3 (PSMA-neg) did not convert (FIG. 5). The high degree of specificity of HBGA conversion was confirmed by testing PC3 cells that had been transfected with PSMA (PC3-PSMA). In these cells which heterogeneously express PSMA, only those cells which were PSMA-pos were converted; adjacent PSMA-neg cells did not convert (FIG. 6).

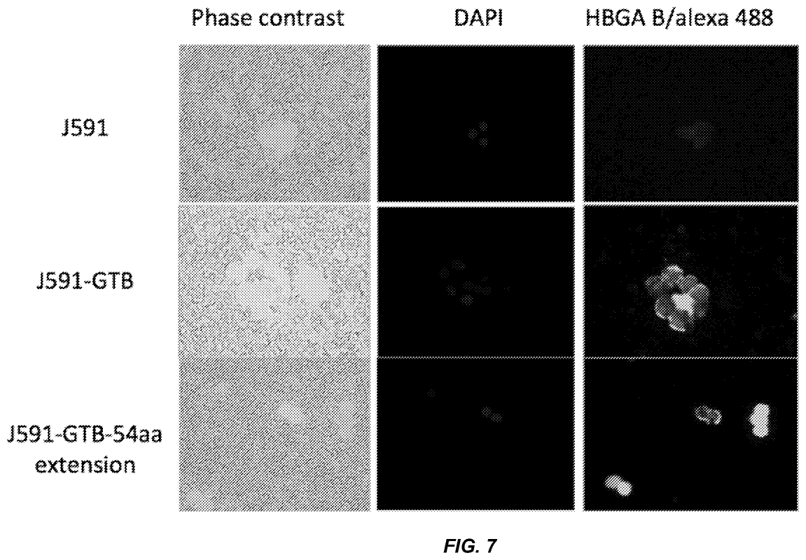

HBGA O LNCaP cells were also co-incubated in type O whole blood plus UDP-Gal and J591 or J591-GTB or J591-GTB-54 amino acid extension. As shown in FIG. 7, while J591 did not convert any cells, J591-GTB, with or without the extension, converted the LNCaP cells, but not the RBCs, from type O to HBGA B.

### Example 4—Lytic Activity of Normal Human O or A Serum After Cancer Cells Conversion to HBGA B

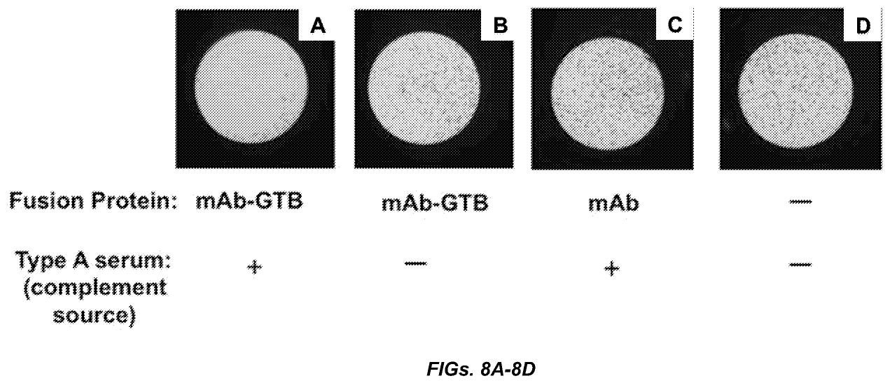

An in vitro assay was used to test the lytic capacity of normal human O and A sera to lyse prostate or breast cancer cells after conversion to HBGA B expression. FIGS. 8A-8D show LNCaP cells (HBGA O) are lysed when incubated with J591-GTB+UDP-gal+human A (or O serum) as a source of anti-B and complement components. Omitting human A or O serum and/or replacing J591-GTB with J591 without GTB resulted in no lysis.

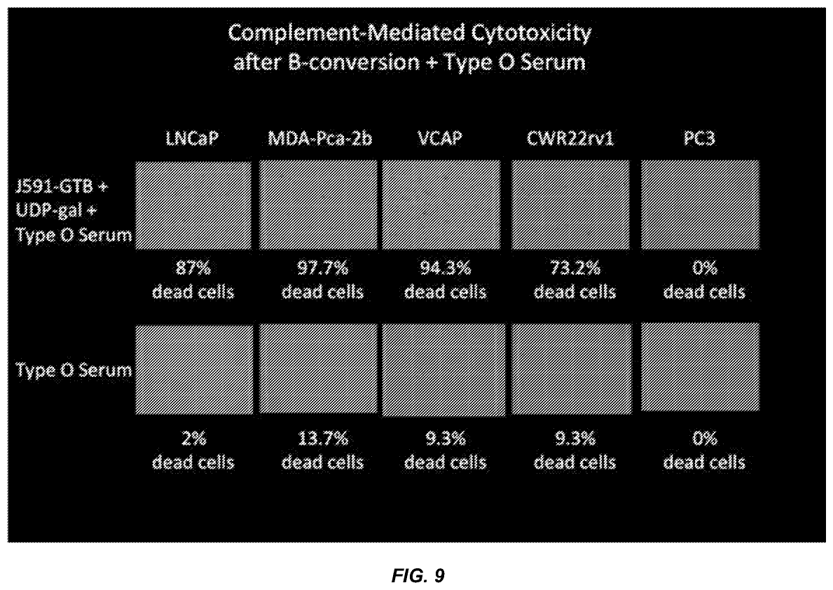

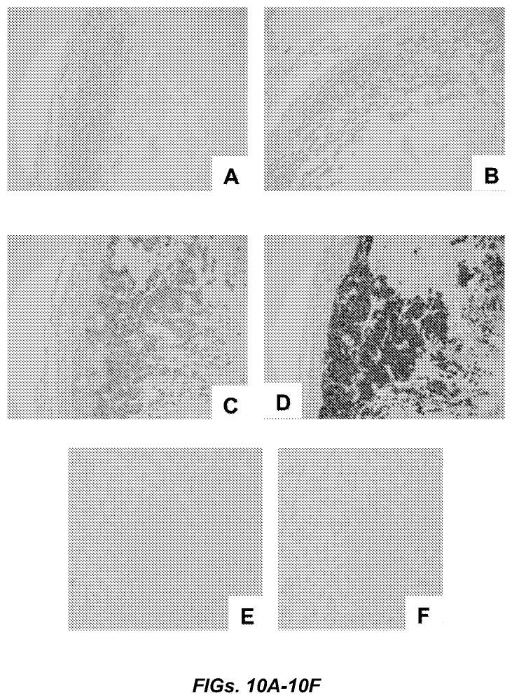

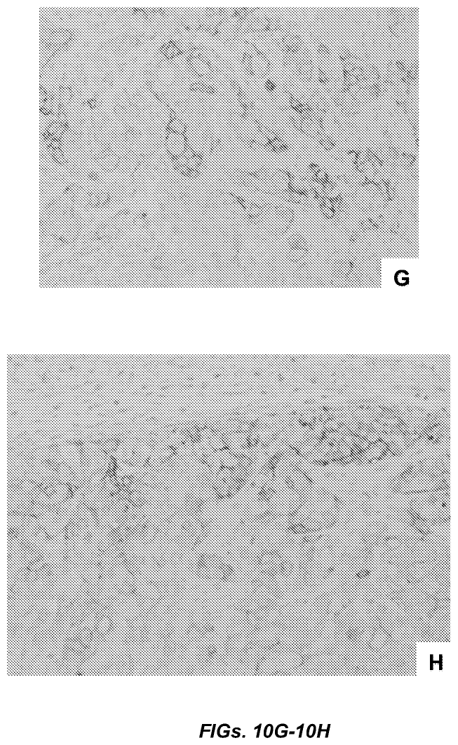

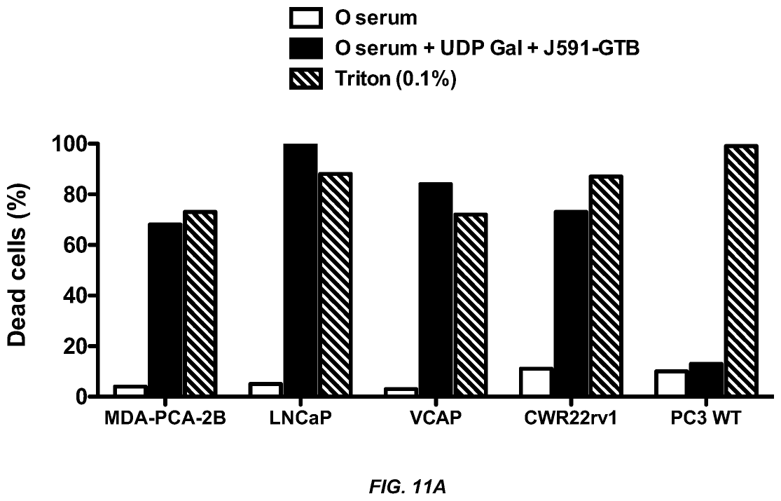

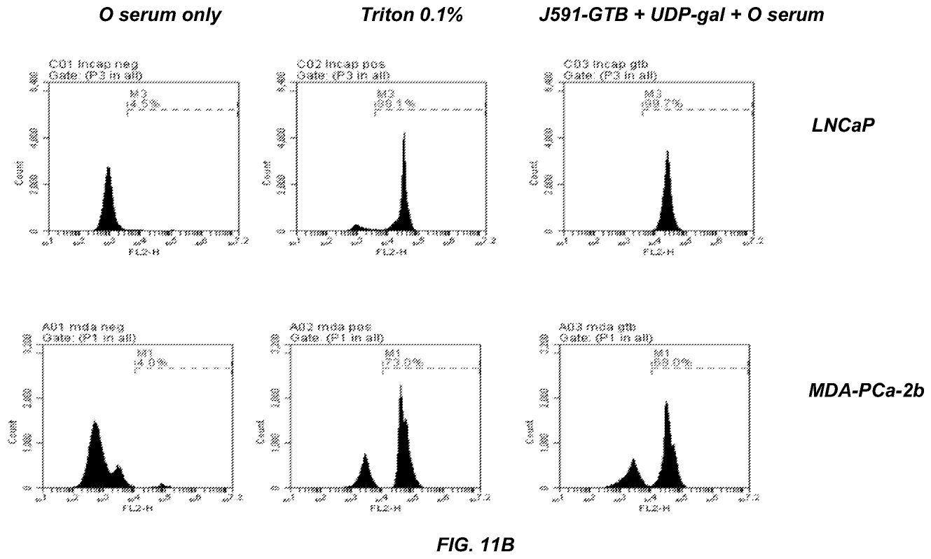

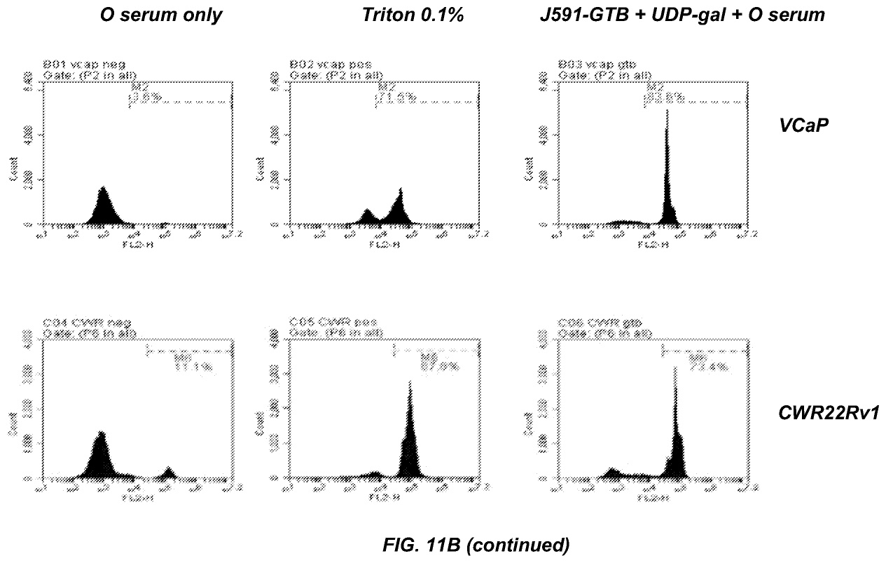

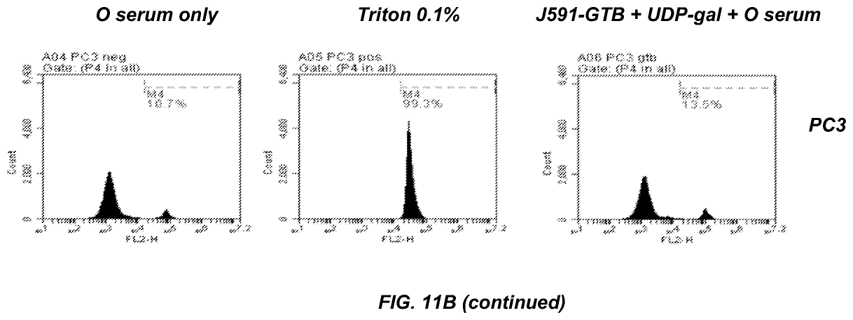

A larger panel of prostate cancer cell lines was assayed, all of HBGA O, both by trypan blue exclusion (FIG. 9) and uptake of propidium iodide by FACS analysis (FIG. 10). Four of these lines (LNCaP, VCaP, MDA-PCa-2b, and CWR22Rv1) express varying levels of PSMA, from high to low, and all were lysed when incubated with J591-GTB+human 0 or A serum containing natural anti-B Ab plus endogenous complement. A 5^{th} cell line, PC3, that is PSMA-neg did not get converted and did not lyse (FIGS. 11A-11B). Similar results were obtained with breast cancer cell line MDA-MB-361 after conversion by the chimeric agent mAb 4D5-GTB.

### Example 5—Conversion of HBGA Expression In Vivo

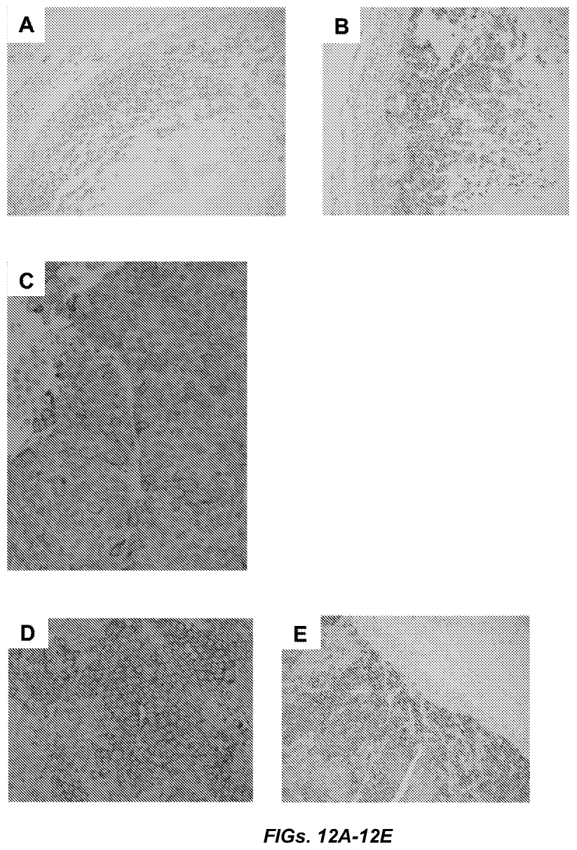

As the rapid rejection and destruction of HBGA-mismatched solid organ transplants is a well-documented and well-established phenomenon in humans since the early days of renal allografts (T. Starzl, Experience In Renal Transplantation. (WB Saunders Company, Philadelphia, PA, chapter 6 (1964); L. Altman, Doctors Discuss Transplant Mistake. New York Times. (2003), which are hereby incorporated by reference in their entirety), the critical in vivo experiment was to demonstrate that the HBGA of an established human cancer could be converted to that of a highly immunogenic HBGA by virtue of a “molecular transplant” of an allogeneic glycosyltransferase, normally functioning within the golgi/ER, to the plasma membrane of the tumor cells using a systemically administered, tumor-targeted approach. For proof of concept, two clinically well-established tumor-associated antigens (FOLH1/PSMA and HER2) derived from 2 of the most common types of solid tumors, prostate and breast cancers, respectively, were selected. Multiple tumor lines expressing a wide range in target expression levels were tested. PSMA-pos prostate cancers LNCaP, C4-2 and CWR22Rv1 and a her2-pos breast cancer, MDA-MB361, were established at subcutaneous sites in NOD SCID mice. J591-GTB or 4D5/trastuzumab-GTB were administered IV; UDP-gal was administered either by IV, IP or subcutaneous route. J591-GTB and 4D5/trastuzumab-GTB converted PSMA-pos prostate cancers and the her2-pos breast cancer, respectively (FIGS. 10A-10H. See also FIGS. 12A-12E).

HBGA B conversion was poor after IP administration of UDP-gal relative to IV or SQ administration. HBGA expression was clearly present at the plasma membrane. As anticipated, replacing Ab-GTB with the respective Ab alone resulted in no HBGA B expression. Conversion was not detectable in any other tissues nor did the animals develop any evidence of toxicity.

### Example 6—Anti-Tumor Activity In Vivo

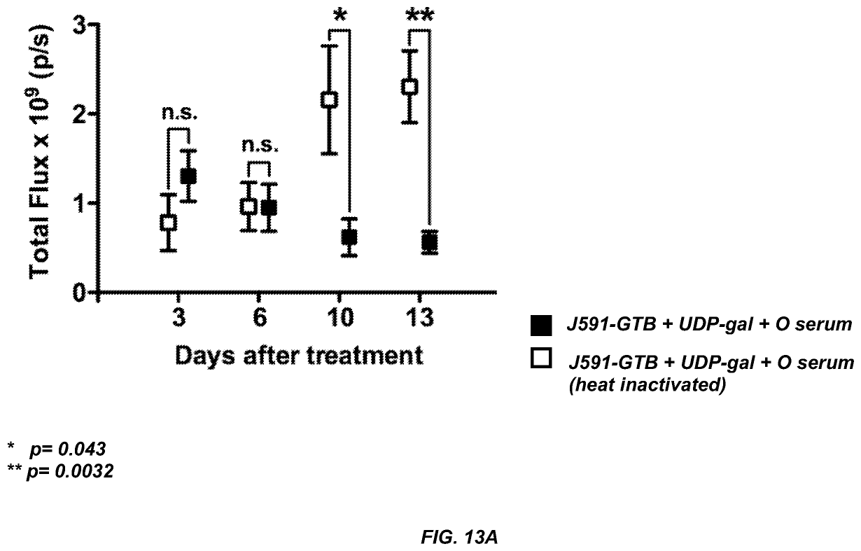

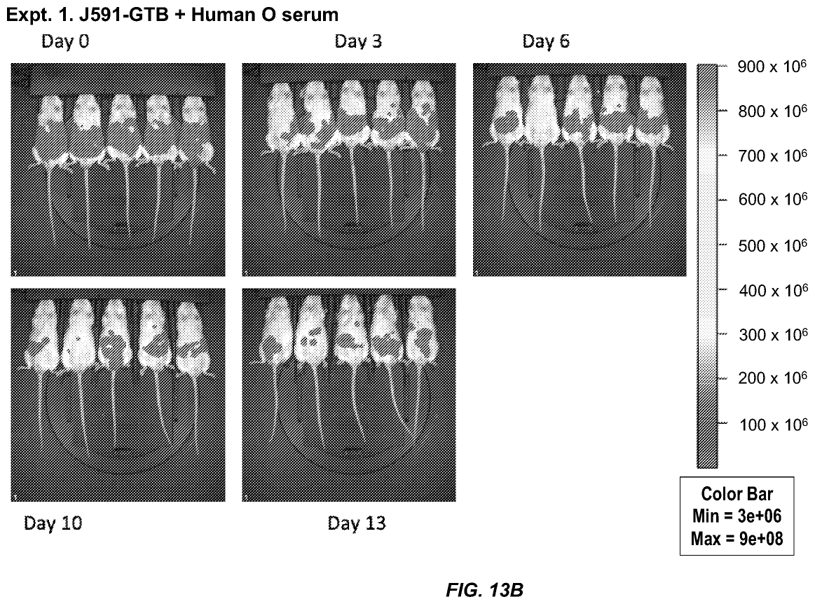

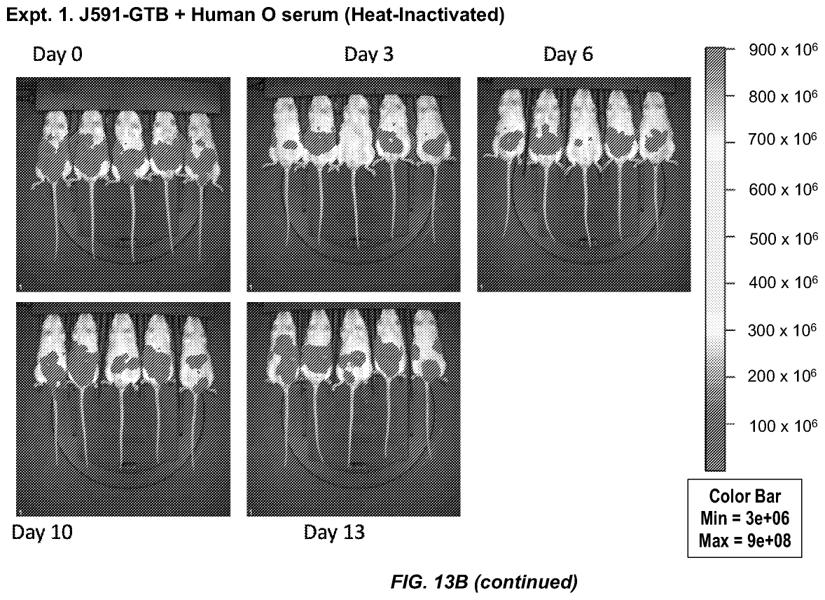

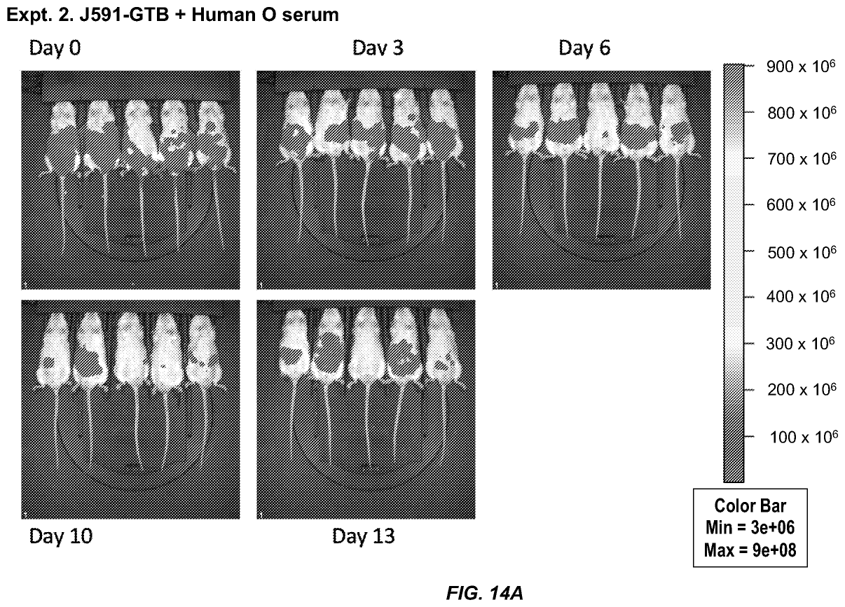

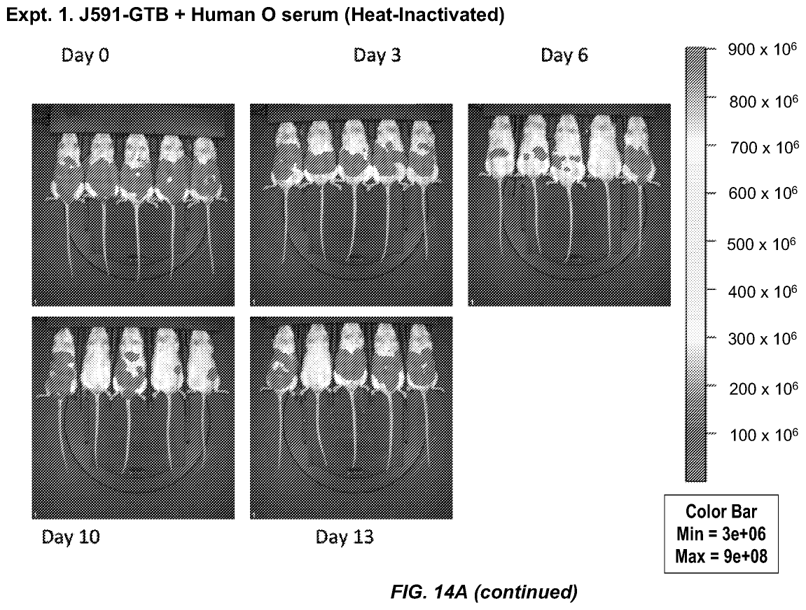

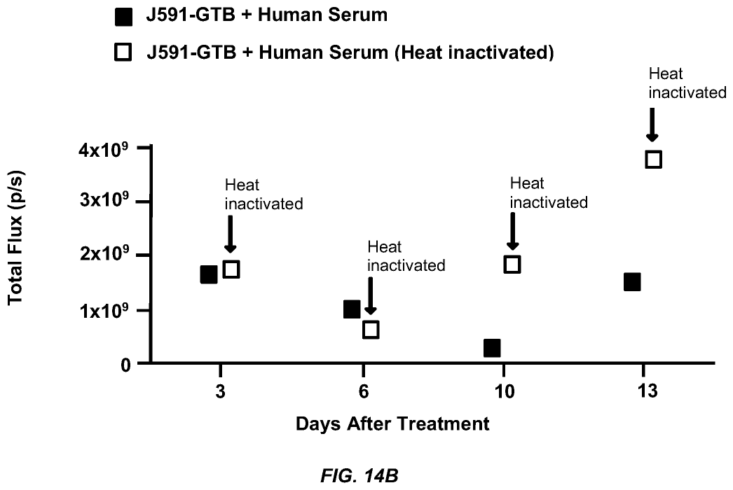

Testing the anti-tumor activity that results from Ab-GTB directed conversion of HBGA expression in an animal model posed several hurdles as both mice and rats express a cis A,B allele as well as the α1,3 GalT allele. As a result, these rodent models are both HBGA A- and B-positive and alpha 1.3 gal-positive, and therefore, tolerant to all of these glyco-structures. In addition, mice have exceptionally weak to inactive complement systems (Bergman et al., Cancer Immunol. Immunother. (2000) 49:259-266; Drake et al., “Passive Administration of Antiserum and Complement in Producing Anti-EL4 Cytotoxic Activity in the Serum of C57BL/6 Mice,” J Natl. Cancer Inst. 50:909-14 (1973); Ong et al., “Mouse Strains With Typical Mammalian Levels of Complement Activity,” J. Immunol. Methods 125:147-158 (1989), which are hereby incorporated by reference in their entirety), in some cases even inhibiting the function of other species', including human, complement activity (Ratelade et al., “Inhibitor(s) of the Classical Complement Pathway in Mouse Serum Limit the Utility of Mice as Experimental Models of Neuromyelitisoptica,” Mol. Immunol. 62:104-113 (2014), which is hereby incorporated by reference in its entirety). Indeed, the complement activity of normal human plasma was assayed in the presence of C57BL/6 plasma and it was found that the human complement lytic activity was reduced by approximately 33%. To overcome the absence of natural Abs and weak, or even inhibitory, complement system in these animal models would require near total replacement of the animals' plasma with human type O or A plasma to provide the necessary natural Abs and functional complement proteins. This plasma replacement is physically impractical, would result in fluid overload, fail to provide the appropriate immunoglobulin bio-distribution equilibrium that reflects the human steady state and be compromised by the inhibitory effect of native mouse plasma. These issues were overcome by developing an intra-peritoneal xenograft model in NOD/SCID mice using the castrate-resistant human PC cell line C4-2-luciferase where human plasma could be injected IP to provide the natural Abs and complement without causing fluid overload. Several days after IP injection of 10×10⁶ C4-2-luc cells and after confirming tumor take by bio-luminescence imaging, 2 groups of 5 animals, each with comparable median/range of bio-luminescent photon flux, received a single IP treatment with J591-GTB, UDP-gal and human type O serum. For the control group, the type O serum was heat-inactivated prior to injection. The total flux of each animal was measured every 3-4 days for approximately 2 weeks. Whereas the animals that received heat-inactivated serum experienced significant tumor progression by day 13, those animals treated with serum containing active complement regressed by 80% relative to the flux of the control group (p=0.0032; FIGS. 13A-13B). A duplicate experiment provided consistent results (FIGS. 14A-14B).

### Discussion of Examples 1-6

The immune response to cancer is strikingly different from the response to an incompatible allograft. As described herein, a strategy is presented to selectively modify tumor cells to express a non-self, highly immunogenic phenotype—incompatible HBGA expression. The swift and destructive result of an HBGA-incompatible allograft in humans was made by Starzl in the early days of renal allografts (T. Starzl, Experience In Renal Transplantation. (WB Saunders Company, Philadelphia, PA, chapter 6 (1964), which is hereby incorporated by reference in its entirety) and led to HBGA compatibility testing as an integral and critical part of donor-recipient matching. Only in those rare cases where an error occurs and the HBGA compatibility requirement is violated is this lesson repeated and reinforced (L. Altman, Doctors Discuss Transplant Mistake. New York Times (2003), which is hereby incorporated by reference in its entirety).

To execute the strategy, clinically validated tumor-restricted antibodies (exemplified by anti-FOLH1/PSMA and anti-HER2) were fused to GTB to produce a single, bi-functional protein. Targeted glycosyltransferases (GTs) have similarly been constructed using a variety of antibody fragments, peptide/ligands, and constructs. While the consequence of incompatible ABO allografts in humans is well established, the challenge in this effort was to molecularly “transplant” the post-translational glycosyltransferase function, normally found in the golgi, to the tumor (or neo-vascular endothelial) cell surface and do so in a systemically administered, tumor-targeted manner.

These chimeric proteins successfully altered the HBGA of a variety of cancer cell lines both in vitro and in vivo. No off-target HBGA conversion or toxicity was seen in the animal experiments. As shown herein, HBGA-incompatible cells trigger complement-mediated lysis, a response that would be predicted to develop in the cancer patient just as it has been demonstrated many times in the clinical transplant setting (L. Altman, Doctors Discuss Transplant Mistake. New York Times (2003); T. Starzl, Experience In Renal Transplantation. (WB Saunders Company, Philadelphia, PA, chapter 6 (1964), which are hereby incorporated by reference in their entirety).

The biosynthesis of the neo-HBGA requires the presence of both the GT and the (fucosylated) H antigen “acceptor structure” on the target cell glycoproteins and glycolipids (Milland et al., “ABO Blood Group and Related Antigens, Natural Antibodies and Transplantation,” Tissue Antigens 68:459-466 (2006), which is hereby incorporated by reference in its entirety) for the HBGA to be added. As a wide array of carcinomas express the H antigen including lung, gastric, colorectal, breast, prostate, ovarian, bladder, pancreas, etc., these tumor types would be candidates for this strategy. Normal, non-target cells do not undergo HBGA conversion due to: (1) lack of binding of the targeted GTB (or GTA) enzyme and (2) absence of the required H Ag from many normal cell types (e.g., bone marrow, liver, spleen, kidney, myocardium, central and peripheral nervous system, etc.) which precludes GTB (or GTA) transferase activity at these sites. FOLH1/PSMA expression has been reported in the tumor neo-vasculature of a wide variety of tumors but absent in normal tissue vasculature. Examples of tumor types that have FOLH1/PSMA-positive neo-vasculature include renal, lung, colon, gastric, breast, brain, pancreatic, hepatic, bladder esophageal, adrenal, head and neck, melanoma and brain tumors, etc. Less commonly but occasionally FOLH1-positive are testicular, lymphoid and sarcomas. Targeting FOLH1/PSMA expression in the tumor neo-vasculature provides a mean to alter the HBGA expression within the vascular bed of a wide variety of tumors. This would, in turn, lead to a similar phenomenon of hyper-acute rejection seen in solid tissue allografts of the wrong HBGA (L. Altman, Doctors Discuss Transplant Mistake. New York Times (2003); T. Starzl, Experience In Renal Transplantation. (WB Saunders Company, Philadelphia, PA, chapter 6 (1964), which are hereby incorporated by reference in their entirety).

The enzymatic nature of the reaction provides an amplification effect as each targeted enzyme molecule converts numerous acceptor molecules. Furthermore, not only are the Ab-targeted tumor-associated antigens themselves enzymatically converted but so are all the neighboring molecules that are within range of the enzyme. And as most cell surface molecules have multiple glycosylation sites—FOLH1/PSMA, for example, has 10 glycosylation sites (20 if one considers that FOLH1/PSMA is normally expressed as a homo-dimer)—the quantity of non-self HBGA sites that can be generated by this approach is very substantial. Furthermore, glycoproteins secreted by the targeted neo-vascular or tumor cells are also subject to HBGA conversion leading to complement activation in the tumor microenvironment thereby enhancing the peri-tumoral immune milieu.

These aforementioned factors-enzyme amplification, conversion of both directly targeted as well as neighboring molecules and secreted glycoproteins, and the multiplier of abundant glycosylation sites-should result in unprecedented levels of highly immunogenic antigen expression by the tumor cells and within the tumor microenvironment even in the case of a weakly expressed tumor-associated antigenic target. As HBGA O and A patients constitute approximately 85% of the population, GTB was utilized in the proof of concept efforts. In addition, as the polyclonal anti-gal activity that precludes xeno-transplantation cross-reacts with HBGA B (Macher et al., “The Gal alpha1,3Gal beta1,4GlcNAc-R (alpha-Gal) Epitope: a Carbohydrate of Unique Evolution and Clinical Relevance,” Biochim. Biophys. 1780:75-88 (2008), which is hereby incorporated by reference in its entirety), induced expression of HBGA B by the tumor and/or its blood supply makes it the target of an unprecedented level of attack by complement-fixing antibodies capable of mediating high levels of inflammation and hyper-acute rejection. The strategy could be extended to cover HBGA O, A and B patients (≈95% of the population) by use of a GT with both A and B activity. This is achievable by a single nucleotide/amino acid change 803G>C (Gly268Ala) of GTA, a mutation that occurs naturally in the so-called cis AB GT and which generates both HBGA A and B. The approach would be applicable to all but AB patients (≈5% of the population) who harbor neither natural anti-A nor -B antibodies.

The method described herein shares many features with, and is complementary to, recent successful immunotherapeutic approaches. Similar to CAR-T and bi-specific Ab approaches that utilize the T-cell lytic machinery, the present approach engages the lytic machinery of the complement cascade. And beyond the direct lytic effect, triggering the complement cascade within the tumor microenvironment serves as a bridge to enhance the potency of the cellular immune response as C3 activates APCs (Baudino et al., “C3 Opsonization Regulates Endocytic Handling of Apoptotic Cells Resulting in Enhanced T-cell Responses to Cargo-Derived Antigens,” Proc. Natl. Acad. Sci. USA 111:1503-1508 (2014); Surace et al., “Complement is a Central Mediator of Radiotherapy-Induced Tumor-Specific Immunity and Clinical Response,” Immunity 42:767-777 (2015), which are hereby incorporated by reference in their entirety) to promote T cell priming (Kopf et al., “Complement Component C3 Promotes T-cell Priming and Lung Migration to Control Acute Influenza Virus Infection,” Nature Med. 8:373-378 (2002), which is hereby incorporated by reference in its entirety). Furthermore, liberation of free C3d, a fragment of C3, has recently been shown to deplete Tregs (via apoptosis), increase infiltration of CD8+ T-cells producing perforin, TNF-α and IFN-7 and decrease PD-1 expression by T cells (Platt et al., “C3d Regulates Immune Checkpoint Blockade and Enhances Antitumor Immunity,” JCI Insight. 2:e90201 (2017), which is hereby incorporated by reference in its entirety). Additionally, activation of the complement system generates chemotactic factors such as C3a and C5a that induce inflammation and recruit inflammatory cells. This would convert a ‘cold’ tumor microenvironment into a ‘hot’ one further aiding the immune response. In sum, the approach described herein offers the potential to expand the breadth and strength of the immune attack on cancer by directly engaging the humoral immune system and the complement cascade and by its role in enhancing the cellular immune response.

### Example 7—B Conversion of MM1-S with Anti-CD19-GTB

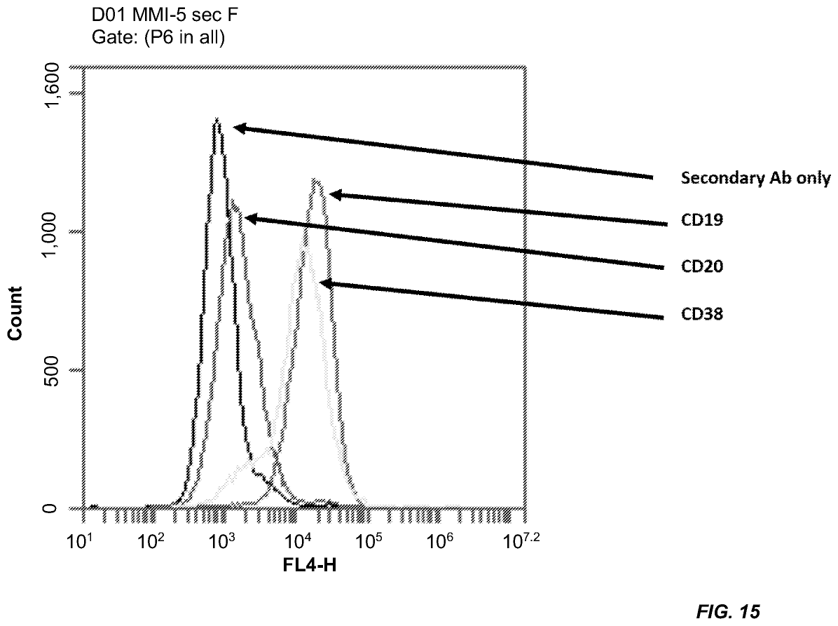

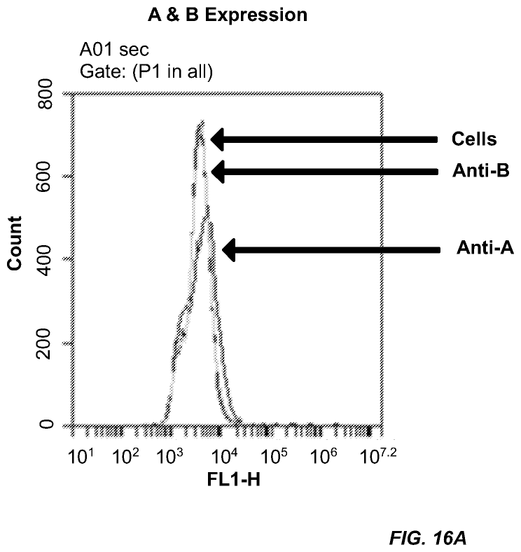

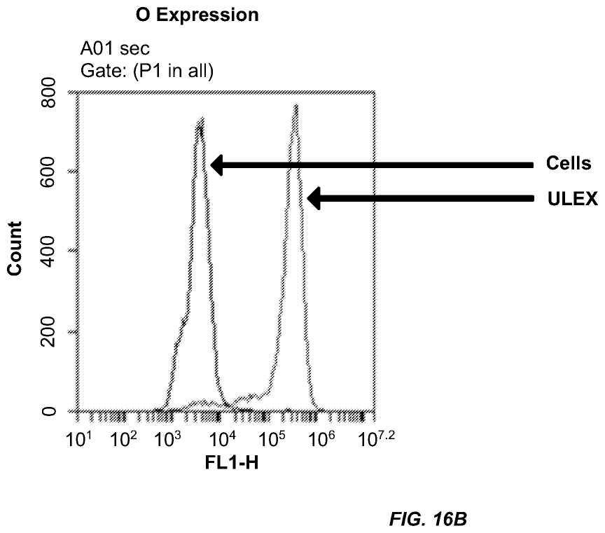

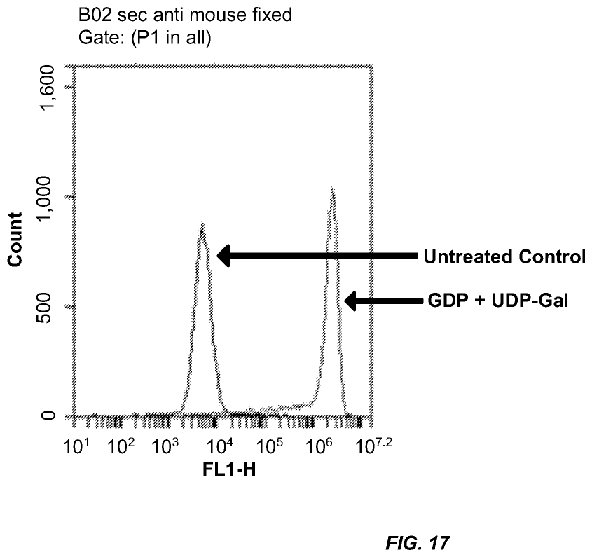

FIGS. 15-17 show the ability to convert CD19-positive/HBGA 0-positive myeloma cells to express HBGA B. In this case, MM1-S myeloma cells that have been passaged in tissue culture were tested by fluorescence-activated cell sorting (FACS) using murine monoclonal antibodies to CD19, CD20, CD38 (FIG. 15), HBGA A, HBGA B (Fisher Scientific (Ortho) and Ulex-FITC or Ulex-Dylight (Vector Labs) to detect HBGA O (FIGS. 16A-16B). MM1-S cells were incubated for 1 hour with each of the antibodies, the cells were washed and then incubated with an appropriate secondary antibody such as anti-mouse IgM-Alexa 488 or 647 (Jackson ImmunoResearch) where the primary antibody was an IgM or a tagged anti-mouse IgG when the primary was an IgG. After another wash, cells were analyzed by FACS. As shown in FIG. 17, the MM1-S cells are incubated with anti-CD19-GTB fusion protein plus UDP-gal, and HBGA B expression is compared by FACS to untreated cells.

FIG. 15 shows MM1-S myeloma cells are CD20-negative, CD19⁺ and CD38⁺. FIGS. 16A-16B shows that MM1-S cells are HBGA A- and B-negative (FIG. 16A) but HBGA O-positive (FIG. 16B). FIG. 17 shows that the MM1-S cells incubated with anti-CD19-GTB fusion protein plus UDP-gal convert to high level HBGA B expression relative to untreated cells.

These experiments provide another example of tumor-targeted conversion to express a foreign antigen: HBGA B. In this case, the target is CD19, a B-cell marker also present on B-cell malignancies. It also represents conversion of a hematogenous tumor type whereas the other examples provided-prostate (PSMA) and breast (HER2) are examples of solid tumors.

### Example 8—Targeting Glycosyltransferase Via a Small Molecule Ligand as an Alternative to Antibody or an Antibody Derivative

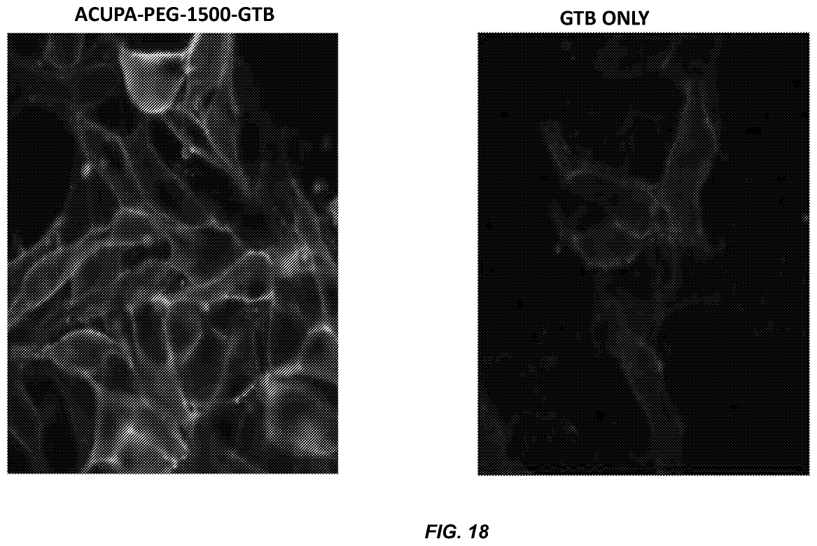

FIG. 18 demonstrates that the targeting of GTA or GTB or alpha-gal can be done, not only by antibody-based constructs but also by a peptide/small molecule ligand-based targeting agent. In this case, the GTB enzyme was conjugated to 2-(3-((S)-5-amino-1-carboxypentyl)ureido) pentanedioic acid (ACUPA), a galactose-urea-lysine-based ligand that binds to PSMA. In order to achieve high level binding, a PEG 1500 spacer was used between the ACUPA and the GTB moieties. This provided adequate steric freedom for the ACUPA to bind the PSMA enzymatic pocket without steric interference from the much larger GTA/GTB enzyme.

FIG. 18 shows that the ACUPA-PEG1500-GTB can convert LNCaP cells from HBGA O to HBGA B (left panel). Use of pure GTB, without the ACUPA moiety for targeting, resulted in no conversion (right panel).

The flexibility to use a variety of targeting moieties, from large antibodies of 150 kd to smaller antibody-derived formats such as monomeric (75 Kd), Fab′2 (100 kd), Fab (50 kd), scFv (25 kd), down to a short peptide such as ACUPA (1.0 kd) enables the construction of fusion proteins with a variety of pharmacokinetic and biodistribution properties. The larger fusion proteins will circulate longer, tend to remain in the blood compartment longer, and be excreted through the liver, whereas the smaller constructs will tend to have shorter serum half-lives, reach/contact the tumor target quicker, and be excreted by the kidney. These various options can be taken advantage of to tailor the therapy depending on the requirements of different tumor types (e.g., hematologic vs solid tumors).

### Example 9—Specificity and Precision of Conversion; Absence of Bystander Effect

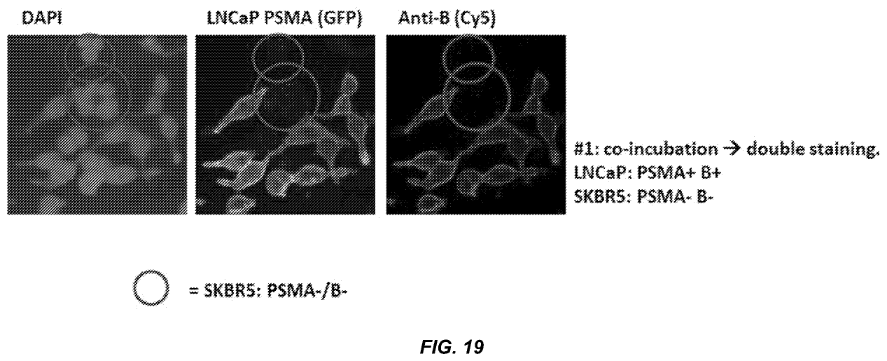

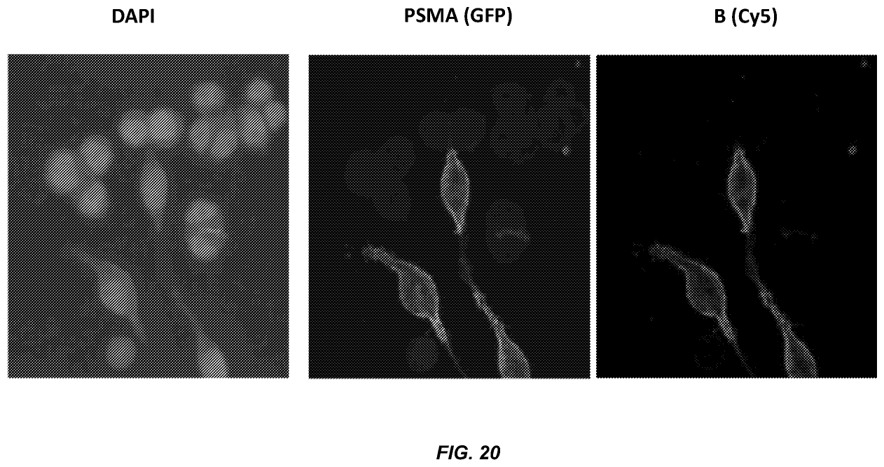

As shown in FIGS. 19 and 20, a breast cancer cell line, SK-BR5 (PSMA-negative) and the LNCaP (PSMA-positive) prostate cancer cell line were co-cultured. The cells can be distinguished as they have distinctive morphologies: SK-BR5 is round and 2 clusters are seen near the center and top (red circles) of the field in FIG. 19. LNCaP is more spindly and these cells also express GFP as an identifying marker.

When the culture was treated with J591-GTB+UDP-gal, the HBGA B (stained with Cy5 (violet)), appears only on the PSMA-positive cells. The neighboring clusters of PSMA-negative SKBR5 cells (highlighted within the red circles) are not converted to HBGA B despite the close proximity to cells that are converted. Similarly, FIG. 20 shows the same distinguishable cell types. The left panel shows all the cell nuclei stained with DAPI. The middle panel shows the spindle shaped LNCaP cells with their green fluorescence due to GFP expression. The right panel, after treatment with J591-GTB+UDP-gal, shows that HBGA B is expressed only by the PSMA-positive LNCaP cells whereas the PSMA-negative SK-BR5 cells remain HBGA B-negative.

This demonstrates both the high degree of specificity as well as the absence of a bystander effect-even neighboring cells are not converted unless they are directly targeted and bind the fusion protein.

### Example 10—Quantifying the Specificity Index

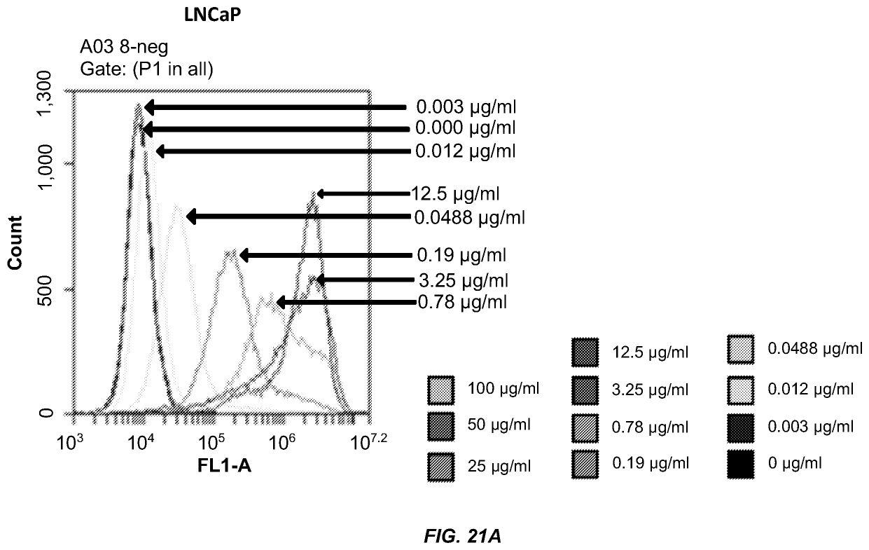

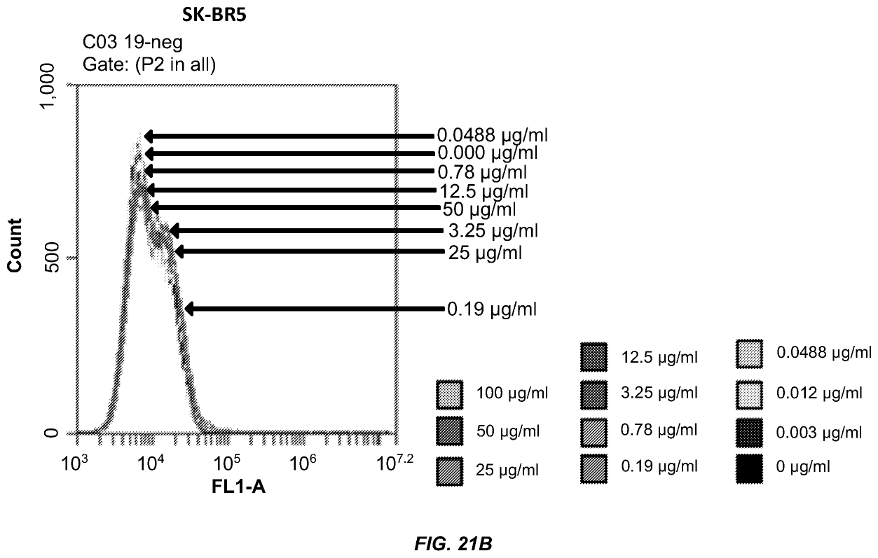

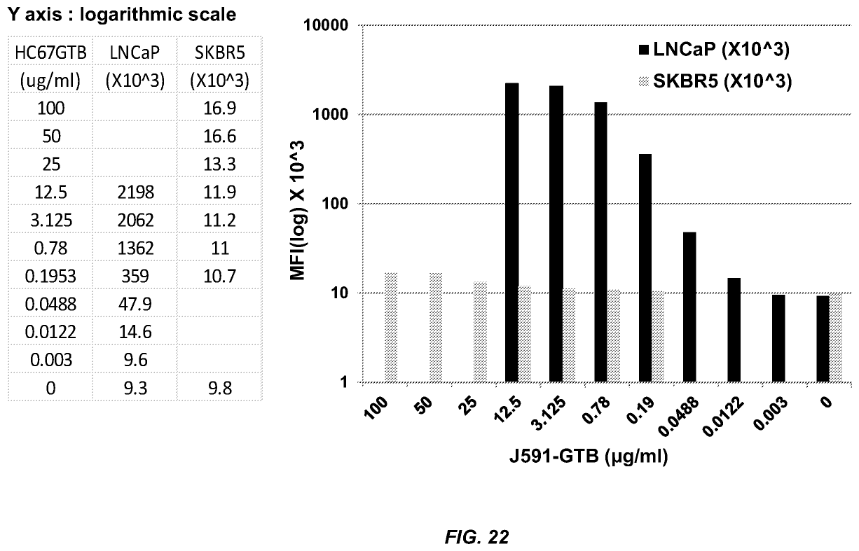

FIGS. 21A-21B and 22 quantitate the specificity index on the same 2 PSMA-positive and -negative cell lines. Different concentrations of anti-PSMA-GTB, from 100 μg/mL down to 0.003 μg/mL were incubated, individually, with each of the cell lines in the presence of the nucleotide donor UDP-gal. The specificity of conversion was quantified using FACS by comparing the concentration of J591 (anti-PSMA)-GTB required to convert LNCaP (PSMA+) to HBGA B relative to SK-BR5 (PSMA-negative) cells. Both cell lines are O+. FACS histograms are shown in FIGS. 21A-21B. Note that concentrations greater than 12.5 ug/mL overlay the 12.5 ug/mL curve and are left off the FACS histogram to simplify viewing.

No B conversion of SK-BR5 occurs even at concentrations of J591-GTB up to 100 ug/mL. By comparison, anti-PSMA-GTB at a concentration as low as 0.012 ug/mL begins to induce the conversion of the PSMA-positive cells. This data is displayed in histogram form in FIG. 22. This indicates that the fusion protein is at least 8,196-fold more specific for PSMA-positive cells than those cells that are PSMA-negative. This results from the ability of the fusion protein to bind directly to PSMA-positive cells where it concentrates at the cell surface and performs its enzymatic function. The enzymatic reaction is far weaker or non-existent where the enzyme does not bind to the targeted cell surface. Concentrations above 100 ug/mL were not tested for reasons of practicality; it is possible that the calculated specificity index is actually much greater than 8,000-fold.

FIGS. 19-22 demonstrate the exquisite specificity of the conversion reaction being limited only to target-positive cells and the lack of a bystander effect whereby even cells that neighbor a converting/target-positive cell are not converted if those cells are target-negative and do not bind the fusion protein.

### Example 11—Both Cell Surface and Secreted Glycoproteins are Glycosylated by this Method

Because both cell surface and secreted glycoproteins are glycosylated by the same cellular processes in the golgi/endoplasmic reticulum, in addition to converting the HBGA of cell surface molecules, glycoproteins secreted by the targeted cell also become HBGA converted. In this exemplary case, the secreted glycoproteins are converted to HBGA B-positive. LNCaP cells were treated with J591-GTB plus UDP-gal for 5 hours (10 ug/ml anti-PSMA-GTB+100 M UDP-gal). As a negative control, another set of LNCaP cells were incubated with 10 g/ml anti-PSMA-GTB but without UDP-gal. As a positive control, measurement of the cell surface conversion was performed with unconverted cells serving as background controls. After the 5 hour incubation, the spent media containing secreted glycoproteins was collected from each set of cells and concentrated 10-fold using an Amicon 3,000 dalton cutoff. The spent media were adsorbed to wells of a plate and assayed by Elisa for the presence of HBGA B using IgM anti-HBGA B followed by anti-mouse IgM-alkaline phosphatase.

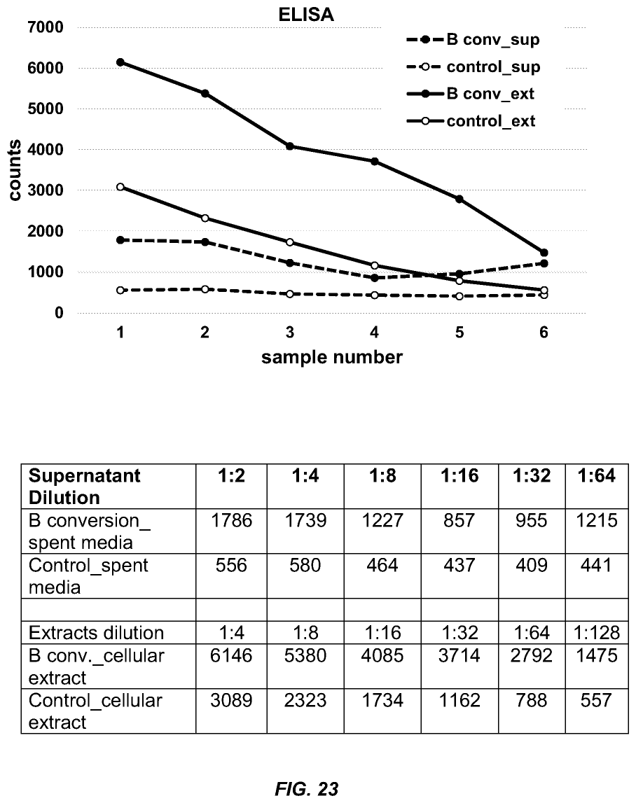

FIG. 23 shows that, relative to the negative control (un-converted spent media), the converted media was positive for the presence of HBGA B on the secreted proteins.

This demonstrates that HBGA B conversion is not limited to the cell surface but also includes glycoproteins secreted by the targeted cells. In vivo, this suggests that these converted, secreted glycoproteins would permeate the tumor extracellular space, be bound by natural anti-B antibody, trigger complement and generate a pro-inflammatory microenvironment, recruit inflammatory and immune cells via chemotaxis and further convert the tumor micro-environment to a ‘hot’ one.

### Example 12—Analysis of the Utilization of Alpha 1,3 Galactosyltransferase (aGalT)

In addition to using human glycosyltransferase A or B enzymes, another embodiment utilizes the enzyme alpha 1,3 Galactosyltransferase (aGalT, EC 2.4.1.87) that is functional in all mammals but is inactive in humans and Old World Monkeys due to evolutionary mutations. GTA, GTB, and alpha 1,3 GalT are highly homologous and thought to have derived from the same ancestral gene. Like GTB, alpha GalT, adds a terminal alpha 1,3 Galactose to the carbohydrate chain of cell surface and secreted glycoproteins and glycolipids, but unlike GTB, alpha GalT can add its Gal in the absence of the H-antigen fucose acceptor structure. As all humans lack a functional alpha GalT and, therefore, lack expression of this terminal alpha 1,3 Gal epitope, they all carry elevated levels of anti-alpha Gal antibodies of the IgM, IgG, IgA and IgE classes estimated to consist of approximately 1% of all circulating immunoglobulin. It is the immunogenicity of this alpha 1,3 Gal epitope that prevents xenotransplantation from other mammals which do have functional aGalT and express the terminal alpha 1,3 Gal on their tissues including their blood vessels.

Use of the alpha GalT abrogates the need to select GTA or GTB depending on the blood type of the subject. It also allows use of this treatment approach in patients who are blood type AB who do not carry natural antibodies to either HBGA A or B but do carry antibodies to alpha 1,3 Gal. Dispensing with the requirement for the H-antigen fucose as an acceptor in the case of alpha GalT also broadens the tissue types that can be addressed. For example, hematopoietic cells and mesenchymal-derived cells (and tumors derived from these cell types), as well as other tissues, lack expression of the H antigen acceptor. These tissues/tumors would not be addressable with GTA or GTB but could be addressed with alpha 1,3 GalT.

One concern with use of alpha Gal Transferase is whether the enzyme itself would be immunogenic in humans given that humans do not express a functional version of the enzyme. If this were the case, repeated administration would require that the enzyme to be humanized or de-immunized. This concern was assessed by assaying sera from 50 randomly selected patients of different blood types to see if any anti-alpha GalT antibodies were present.

An ELISA assay was performed by coating α1,3GalT (500 ng/ml) in a 96-well Half Area High Bind Microplate overnight at 37° C. Negative control wells were not coated with the enzyme. After washing and blocking with PBS-HSA (5%), sera from 50 different donors were added to the plate for 2 hours at room temperature (RT). This included sera from 21 O, 20 A, 3 B, and 6 AB type patients. After washing, anti-α1,3GT antibodies were detected by adding anti-human IgG+IgM-Alk Phos antibody solution followed by adding PNPP and reading the plate at 405 nm. To ensure that α1,3GT was correctly coated, the protein was detected by using an anti His-Tag antibody as the α1,3GT was labelled with a His tag (positive control). As another control, to ensure that the AP anti-human IgG+IgM was functional, BSA was coated at 500 ng/ml and anti-BSA antibodies from human serum (HBGA A) were measured using the same method.

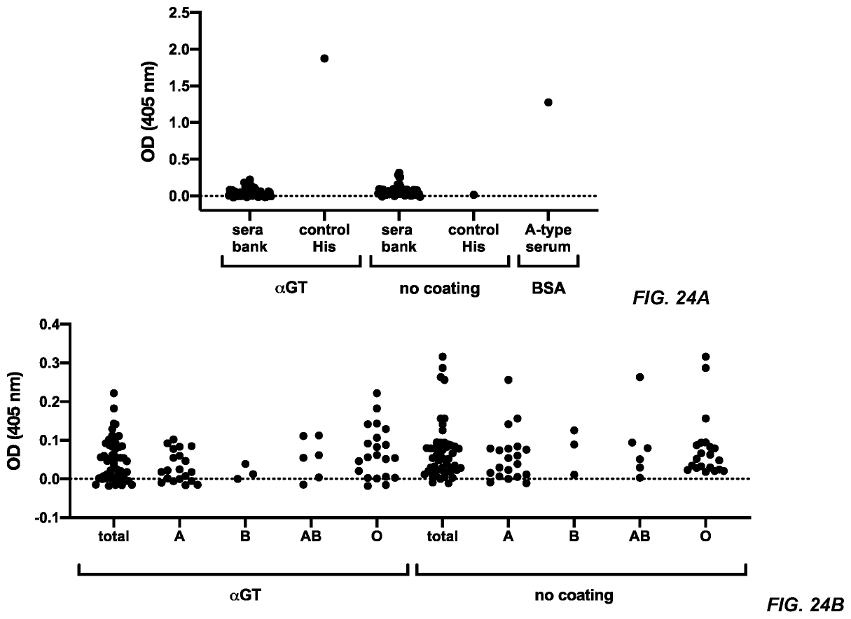

It was found that none of the 50 sera contained antibodies to alpha 1,3 GalT (FIGS. 24A-24B). It is presumed that this is due to the homology of the wide variety of glycosyltransferases including, but not limited to, GTA and GTB. This result indicates that one can use the alpha GalT enzyme in a fusion protein, in order to generate the alpha Gal epitope, without concern that the fusion protein would be immunogenic. Therefore, it is unlikely to require de-immunization or humanization.

### Example 13—Expression and Purification of Recombinant Alpha 1,3 Galactosyltransferase (aGalT)

An anti-CD19 scFv fused to a portion of the alpha 1,3 GalT sequence (aa90-376) was constructed, analogous to the approach with GTA and GTB. The scFv sequences used were derived from Denintuzumab (Den) from Seattle Genetics and Obexelimab (Obx) from Xencor which recognize and bind to both cynomolgus and human CD19. The same (G₄S)₃ spacer/linker as previously described in the construction of Ab-GTB was used. For the alpha GalT, the marmoset sequence was chosen which has 376 amino acid residues and is consistent with the general topology of glycosyltransferases: 6 aa cytoplasmic domain, 16 aa transmembrane domain, and 354 aa in the luminal domain containing the enzymatic activity.

The stem region of marmoset α1,3GT is comprised of 67 amino acids and spans amino acids 23-89 of the luminal portion of the enzyme; it can be removed without affecting enzyme activity. A truncated 90-376 α1,3GT is functional and was selected for the fusion protein. A His tag was added to the enzyme. The construct was expressed in Expi293F cells and purified using a metal affinity column.

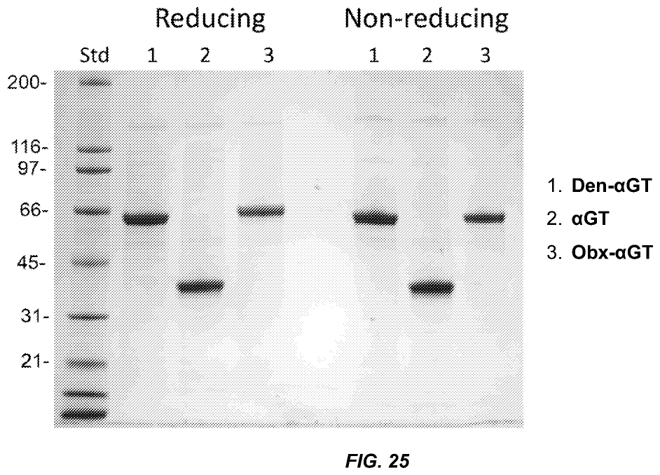

SDS-PAGE electrophoresis probed with anti-his revealed highly pure preparations of the desired constructs at their appropriate, predicted molecular weights (FIG. 25).

### Example 14—Examination of the Functionality and Specificity of the Anti-CD19 scFv-alpha GalT Constructs

To demonstrate the functionality and specificity of the anti-CD19 scFv-alpha GalT constructs, a hematopoietic target was chosen that does not express either the H-antigen acceptor structure or HBGA A or B: CD19 on Raji B-lymphoma cells. CD19 is also a validated tumor target. In addition to functionality, specificity was assessed by comparing the alpha Gal addition to CD19+ Raji cells co-incubated with CD19-neg MM1.S cells.

CD19-positive cancer cell line Raji-GFP was mixed with CD19-negative cancer cells (MM1.S) at different ratios and incubated with the scfv-αGalT constructs (10 μg/ml) and UDP-Gal (5 mM) for 1 hour at 37° C. The presence of α1,3Gal epitopes was then assessed by flow cytometry using an anti-α1,3Gal antibody; Raji-GFP cells were used to differentiate them from MM1.S cells.

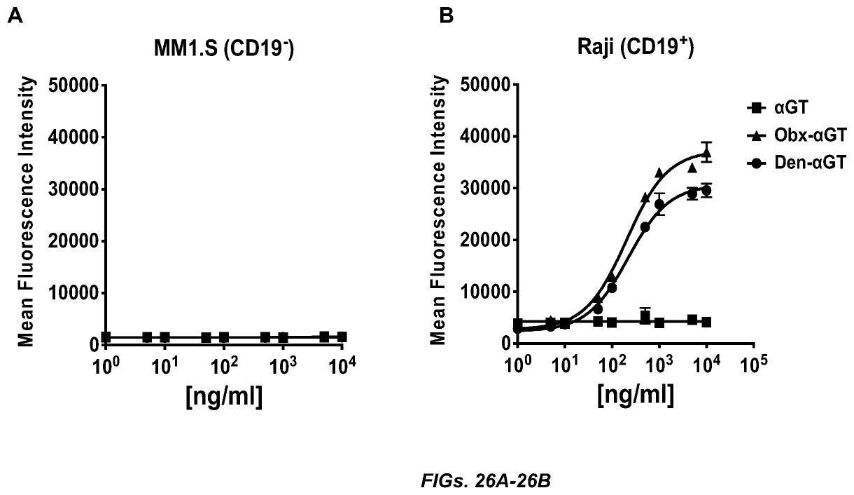

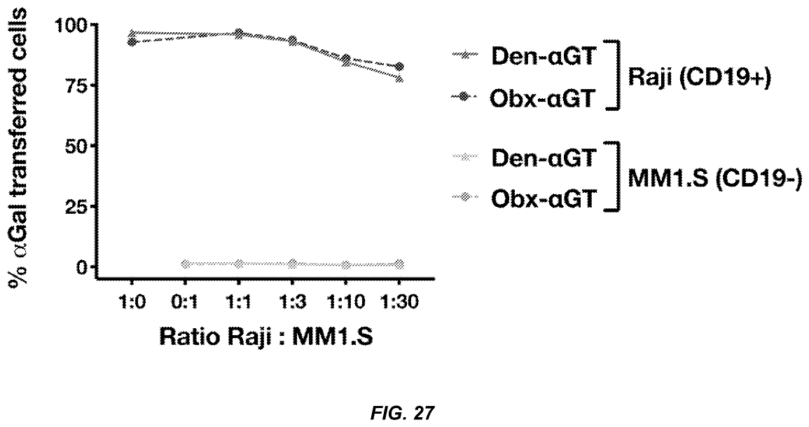

The fusion protein binds to CD19-positive Raji cells saturating at 1-10 g/mL but does not bind to the CD19-negative MM1.S cells (FIGS. 26A-26B). The anti-CD19 scFv-αGalT constructs added a terminal alpha 1,3 Gal to CD19-pos Raji cells but not to CD10-neg MM1.S cells (FIG. 27) even when the latter was present at a 30-fold excess to the former. MM1.S, even at high ratio to Raji cells, never became αGal positive in presence of scfv-αGalT fusion proteins+UDP-Gal.

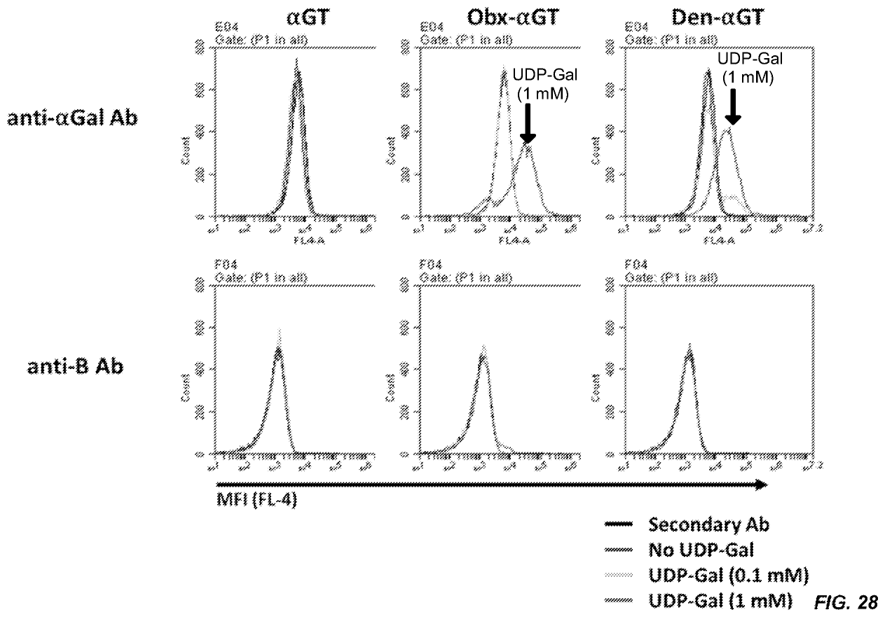

The alpha 1,3 GalT itself, without the scFv binding domain, does not add the Gal moiety demonstrating that binding via the antibody (or fragment) moiety of the fusion protein is required for adding the alpha 1,3 Gal (FIG. 28). In addition, when UDP-gal was not added, no Gal was added to the target cells. In the presence of UDP-gal, the alpha 1,3 Gal moiety was added, but it does not generate a HBGA B epitope as demonstrated by the lack of binding by an antibody to HBGA B (FIG. 28).

### Example 15—Ability of the Anti-CD19 scFv-aGalT to Convert Fresh Human Lymphocytes

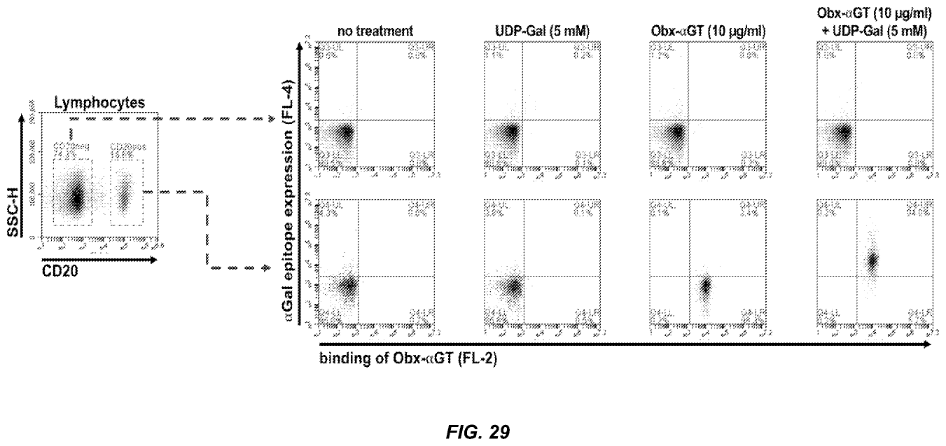

Similarly, the ability of the anti-CD19 scFv-aGalT to convert fresh human lymphocytes was tested. To avoid distorting results due to the presence of an anti-CD19 construct, anti-CD20 was used to identify the B-cells in this experiment. Cells were incubated with UDP-gal only, Obx CD19-alpha GalT only, or both Obx CD19-alpha GalT plus UDP-gal at the concentrations shown. Two channel FACS was used to measure both binding of the CD19-alphaGalT (X-axis) and expression of the alpha Gal epitope (Y-axis) (FIG. 29). A no treatment negative control was also run.

CD20-negative cells did not bind the fusion protein nor were they converted to express alpha 1,3 Gal (FIG. 29, upper panel). CD20-positive cells (FIG. 29, lower panel) demonstrated binding of the fusion protein and were converted to express alpha 1,3 Gal only when UDP-gal was also added.

### Example 16—Lytic Functionality of Human Sera from Different Blood Group Donors on CD19-positive Raji-GFP Cells

The lytic functionality of human sera from different blood group donors on CD19-positive Raji-GFP cells was tested. CD19+ Raji-GFP cells were incubated with Obx-αGT (10 μg/ml) with or without UDP-Gal (5 mM) for 1 hour at 37° C. Sera from different donors were then added to the cells and incubated for 4 hours at 37° C. Viability of the cells was assessed by flow cytometry by measuring their GFP expression.

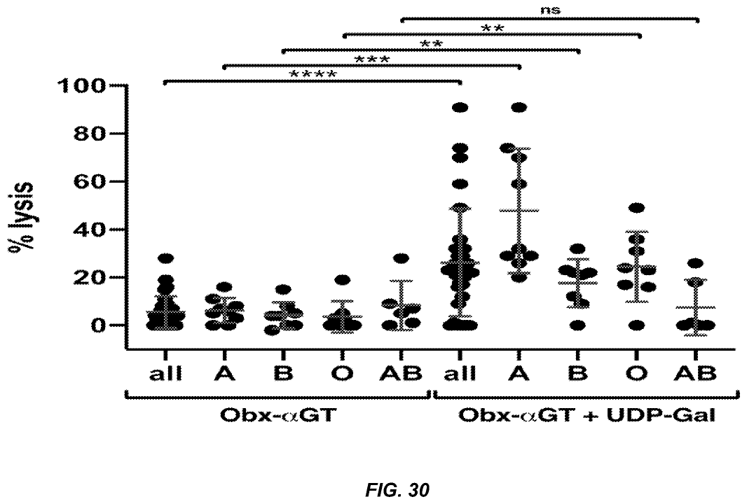

Unless UDP-gal was provided as a nucleoside donor to complete the anti-CD19 scFv-αGalT conversion to express the terminal alpha 1,3 Gal, only background lysis was seen (FIG. 30). But when UDP-gal was included and the alpha gal conversion took place, human sera lysed the converted cells. In this experiment, it was found that type O and A sera caused greater lysis than either type B or AB sera.

### Example 17—Conversion and Lysis of Fresh Human B-Cells Using Autologous Sera

The above was extended to investigate conversion and lysis of fresh human B-cells using autologous sera (from the same donor) where the individual donor's level of anti-alpha 1,3 Gal IgG and IgM levels were also measured. Human PBMCs from donors of different blood types were incubated with Obx-αGT (10 μg/ml) and UDP-Gal (5 mM) for 1 hour at 37° C. An aliquot of cells was analyzed by FACS for binding of human IgG and human IgM using anti-gamma or anti-mu chain-specific antibodies. Sera from the same donors was then added to another aliquot of the cells and incubated for 4 hours at 37° C. B-cell depletion was measured by flow cytometry using anti-CD20.

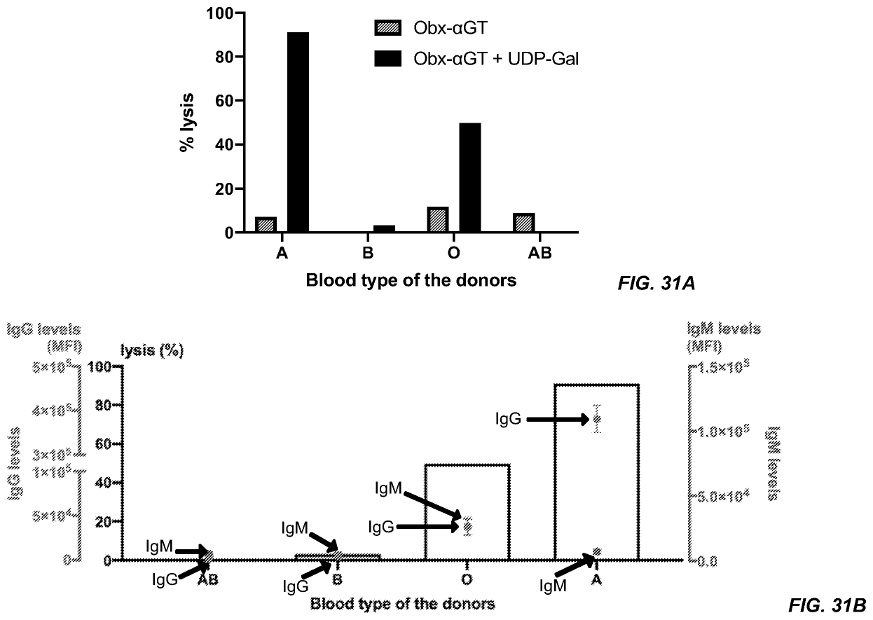

The greatest degree of lysis of converted cells was found by patients of type A and O, and this corresponded with the individual patient's level of anti-alpha 1,3 Gal (FIGS. 31A-31B). This suggests that measuring anti-alpha gal prior to treatment will allow prediction of patients more or less likely to respond to this treatment approach. It also suggests that some patients, particularly type B or AB, may benefit from priming by exposure to the alpha 1.3 gal antigen to stimulate a higher level of anti-alpha gal antibodies. This could occur by administering an alpha gal containing polysaccharide or glycoprotein subcutaneously at least a week prior to this therapeutic approach or, alternatively, the initial treatment cycle's induction of the alpha gal epitope can serve to stimulate production of anti-alpha gal antibodies to be present for subsequent cycles. One knowledgeable in the art can assess a series of patients for their pre-treatment anti-alpha gal levels and their relationship to response and determine a threshold below which response is less likely without priming.

### Example 18—Determination of Optimal Concentrations of Anti-CD19 scFv-aGalT and UDP-gal to Generate Human Donor CD19 Cell Lysis Using Autologous Serum

The optimal concentrations of the anti-CD19 scFv-aGalT and UDP-gal to generate human donor CD19 cell lysis using autologous serum was determined. Human PBMCs from a HBGA type-A donor known to induce serum-mediated lysis on αGal converted cells were incubated with Obx-αGT and UDP-Gal at different concentrations for 1 hour at 37° C. Serum from same donor was then added to the cells and incubated for 4 hours at 37° C. Binding, α1,3Gal transfer and B-cell depletion were assessed by flow cytometry.

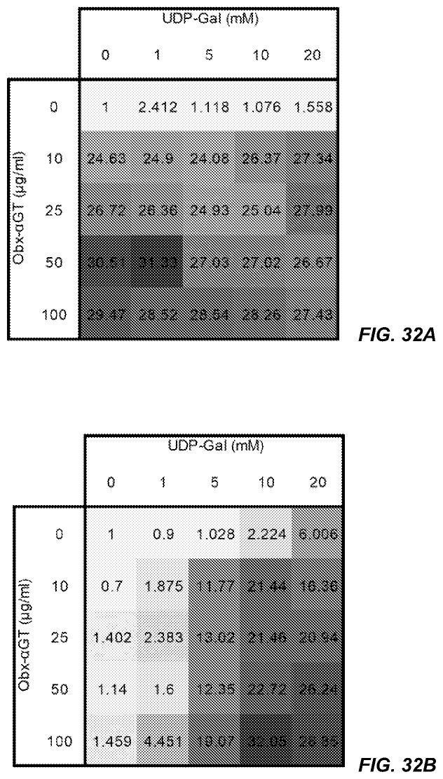

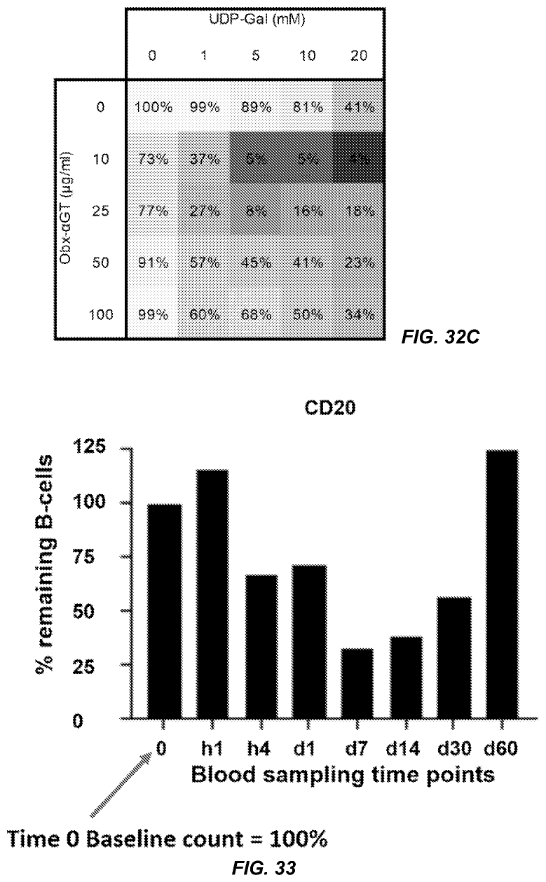

The anti-CD19 scFv-aGalT saturated the CD19 cells at approximately 10 ug/mL (FIGS. 32A-32C). Expression of alpha 1.3 gal was best at a UDP-gal concentration of approximately 10 mM. B-cell lysis was maximal with anti-CD19 scFv-aGalT at 10 ug/mL and UDP-gal in the range of 5-20 mM. B-cell lysis progressively diminished at concentrations of anti-CD19 scFv-aGalT>10 ug/mL and especially >/=25 ug/mL, above the saturation point of CD19. This is likely due to unbound anti-CD19 scFv-aGalT competing for UDP-gal thereby diminishing available UDP-gal for the cell bound anti-CD19 scFv-aGalT.

The concentrations of scFv-aGalT and UDP-gal may vary depending on the cancer target antigen, its density on the tumor cell membrane and lysis efficacy may vary depending on the level of anti-alpha 1,3 Gal antibodies (IgM and/or IgG and/or IgA and/or IgE). All of these parameters can be measured pre-treatment, and one of skill in the art may determine the optimal concentrations of the various components for treatment of each individual patient.

### Example 19—Engineering of Anti-CD19 scFv-alpha Gal Transferase

Obexelimab-scFv-α-1,3 Gal (SEQ ID NO: 63) was constructed by fusing an Obexelimab single chain variable fragment (scFv) in vH-vL orientation to the N-terminus of Marmoset derived α-1,3 galactosyltransferase (aa90-376) via an (G₄S)₃ linker. A 6His tag was added to the C-terminus of the fusion protein to enable affinity chromatography purification.

The generation of the protein was carried out at WuXi Biologics. Briefly, the target DNA sequence encoding Obexelimab-scFv-α-1,3 Gal (SEQ ID NO: 63) was codon optimized, synthesized, and subcloned into WuXi Biologics' proprietary expression vector. The fusion protein was expressed by transient transfection in CHO cells scaled up to 2 L. Obexelimab-scFv-α-1,3 Gal was purified from cell culture supernatant by a three step column purification process. Nickel affinity chromatography was used in the initial captured step, followed by anion-exchange chromatography and then size exclusion chromatography to obtain 95% protein purity with endotoxin levels<1 EU/mg. The purified protein was formulated in histidine buffer pH 6.0 at 20 mg/ml. Protein purity was evaluated by SDS-PAGE and SEC-HPLC and endotoxin level were tested.

### Example 20—In Vivo Treatment of a Non-Human Primate with Anti-CD19 scFv-alpha Gal Transferase Fusion Protein Plus UDP-Gal

Next, whether in vivo treatment of a non-human primate with anti CD19 scFv-alpha Gal transferase fusion protein plus UDP-Gal leads to reduction of CD19⁺ B-cell counts or toxicity was investigated.

Two cynomolgus monkeys (each 5 kg body weight) underwent baseline blood tests to confirm acceptable laboratory values and to measure baseline B-cell and T-cell counts. Next, the cynomolgus monkey received an intravenous injection of anti-CD19 scFv-alpha Gal transferase (SEQ ID NO: 63) at time 0 followed by an injection of UDP-gal.

Complete blood counts, serum chemistries, liver function tests, and total lymphocyte, B-cell counts, and T-cell counts were measured at 1 hour, 4 hours, and 24 hours, and at days 7, 14, 30, and 60 post-treatment (FIG. 33). B-cell counts were determined by examining the CD20⁺/CD3⁻ fluorescence. CD20 was used to avoid confounding the B-cell count by presence of anti-CD19 scFv.

Treatment of the first subject monkey with anti-CD19 scFv-alpha-Gal Transferase plus UDP-Gal led to a 70% reduction in CD19/CD20⁺ B-cells at 7 days post-treatment that lasted 8 weeks before returning to its baseline level. The second monkey, that more recently received a higher dose of the anti-CD19 scFv-alpha-Gal Transferase, had an 80% B-cell decline at the first measurement done at 4 hours. The subject monkeys had no visible signs of toxicity observed by veterinarians. The subject monkeys' weight remained unchanged. They had had no measurable signs of toxicity on blood testing. In the first monkey, blood tested lab values, other than the CD19/CD20 counts remained stable during the 2 month follow up. The second monkey has data only up to 48 hours and she is still being studied.

These results demonstrate that a non-human primate can safely be treated by the methods disclosed herein and that the treatment leads to a substantial reduction in the targeted cells.

### Example 21—Engineering of Tumor-Targeted Bi-Functional Therapeutic Protein

Tumor-targeted fusion proteins were constructed by genetically fusing the H chain of tumor targeting antibody to a glycosyltransferase enzyme. In the examples described herein, GTA (SEQ ID NO: 64) and GTB (SEQ ID NO: 65) are derived from their known human sequences while αGalT is derived from the marmoset sequence (SEQ ID NO: 66). The post-translational enzymes used in the examples presented herein were all naturally expressed in the Golgi and/or endoplasmic reticulum vesical membranes. For construction of the bi-functional fusion proteins described herein, the portions of the enzymes that are not necessary for enzymatic function (e.g., the extra-vesical, transmembrane and stem regions) have been omitted.

As described herein supra, the tumor-targeting portion of the fusion protein can be full length Ab, Fab′2, Fab, scFv, monomeric Ab or any Ab/immunoglobulin derivatives thereof. The enzymatic portion of the fusion proteins can be any post-translational modifying enzyme; its sequence will generally be human, humanized, primatized (from non-human primate) or otherwise deimmunized. The attachment of targeting moiety to enzyme may be with or without a linker/spacer. In the examples provided herein, the (G₄S)₃ (SEQ ID NO: 67) linker/spacer is used but any linker/spacer known to those in the art may be used.

The preparation of these fusion proteins is modular so any tumor/tissue targeting moiety may be fused to any post-translational enzyme following the several examples provided herein. Sequences used in the engineering and generation of huJ591 and 4D5 bi-functional therapeutics are provided in Table 7.

TABLE 7
Sequences

| | | SEQ ID |
|---|---|---|
| Protein Sequence | Sequence | NO: |
| huJ591 heavy | EVQLVQSGPEVKKPGATVKISCKTSGYTFTEYTIHWVKQAPGKGLEW | 68 |
| chain | IGNINPNNGGTTYNQKFEDKATLTVDKSTDTAYMELSSLRSEDTAVYY | |
| | CAAGWNFDYWGQGTLLTVSSASTKGPSVFPLAPSSKSTSGGTAALG | |
| | CLVKDYFPEPVTVSWNSGALTSGVHTFPAVLQSSGLYSLSSVVTVPS | |
| | SSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAV | |
| | EWESNGQPENNYKTTPPVLDSDGSFFLYSKLTVDKSRWQQGNVFSC | |
| | SVMHEALHNHYTQKSLSLSPGK | |
| N-Terminus of | EPDHLQRVSLPRMVYPQPKVLTPCRKDVLVVTPWLAPIVWEGTFNIDI | 69 |
| Human GTB (aa | LNEQFRLQNTTIGLTVFAIKKYVAFLKLFLETAEKHFMVGHRVHYYVFT | |
| 57-354) | DQPAAVPRVTLGTGRQLSVLEVGAYKRWQDVSMRRMEMISDFCER | |
| | RFLSEVDYLVCVDVDMEFRDHVGVEILTPLFGTLHPSFYGSSREAFTY | |
| | ERRPQSQAYIPKDEGDFYYMGAFFGGSVQEVQRLTRACHQAMMVD | |
| | QANGIEAVWHDESHLNKYLLRHKPTKVLSPEYLWDQQLLGWPAVLR | |
| | KLRFTAVPKNHQAVRNP | |
| 6 his tag | HHHHHH | 70 |
| huJ591-LC | DIQMTQSPSSLSTSVGDRVTLTCKASQDVGTAVDWYQQKPGPSPKL | 71 |
| | LIYWASTRHTGIPSRFSGSGSGTDFTLTISSLQPEDFADYYCQQYNSY | |
| | PLTFGPGTKVDIKRTVAAPSVFIFPPSDEQLKSGTASVVCLLNNFYPR | |
| | EAKVQWKVDNALQSGNSQESVTEQDSKDSTYSLSSTLTLSKADYEK | |
| | HKVYACEVTHQGLSSPVTKSFNRGEC | |
| huJ591 heavy | EVQLVQSGPEVKKPGATVKISCKTSGYTFTEYTIHWVKQAPGKGLEW | 72 |
| chain (VH-CH1- | IGNINPNNGGTTYNQKFEDKATLTVDKSTDTAYMELSSLRSEDTAVYY | |
| partial hinge | CAAGWNFDYWGQGTLLTVSSASTKGPSVFPLAPSSKSTSGGTAALG | |
| sequence) | CLVKDYFPEPVTVSWNSGALTSGVHTFPAVLQSSGLYSLSSVVTVPS | |
| | SSLGTQTYICNVNHKPSNTKVDKKVEPKSCDKTHT | |
| Myc-his tag | AAAEQKLISEEDLNGAVEHHHHHH | 73 |
| huJ591 heavy | EVQLVQSGPEVKKPGATVKISCKTSGYTFTEYTIHWVKQAPGKGLEW | 74 |
| chain | IGNINPNNGGTTYNQKFEDKATLTVDKSTDTAYMELSSLRSEDTAVYY | |
| | CAAGWNFDYWGQGTLLTVSSASTKGPSVFPLAPSSKSTSGGTAALG | |
| | CLVKDYFPEPVTVSWNSGALTSGVHTFPAVLQSSGLYSLSSVVTVPS | |
| | SSLGTQTYICNVNHKPSNTKVDKKVEPKSCDKTHTVPPVPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTKPPSRDELTKNQVSLSCLVKGFYPSDIA | |
| | VEWESNGQPENNYKTTVPVLDSDGSFRLASYLTVDKSRWQQGNVF | |
| | SCSVMHEALHNHYTQKSLSLSPGK | |
| | (HC67 variant amino acids (n = 8) shown in bold.) | |
| 54aa tail | EFEQKLISEEDLNSADIHHTGARSSAHLELTADYKDHDGDYKDHDIDY | 75 |
| | KDDDDK | |
| huJ591scFv/Fc | DIQMTQSPSSLSTSVGDRVTLTCKASQDVGTAVDWYQQKPGPSPKL | 76 |
| | LIYWASTRHTGIPSRFSGSGSGTDFTLTISSLQPEDFADYYCQQYNSY | |
| | PLTFGPGTKVDIKEVQLVQSGPEVKKPGATVKISCKTSGYTFTEYTIH | |
| | WVKQAPGKGLEWIGNINPNNGGTTYNQKFEDKATLTVDKSTDTAYM | |
| | ELSSLRSEDTAVYYCAAGWNFDYWGQGTLLTVSSEPKSCDKTHTVP | |
| | PVPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKC | |
| | KVSNKALPAPIEKTISKAKGQPREPQVYTKPPSRDELTKNQVSLSCLV | |
| | KGFYPSDIAVEWESNGQPENNYKTTVPVLDSDGSFRLASYLTVDKSR | |
| | WQQGNVFSCSVMHEALHNHYTQKSLSLSPGK | |
| | (HC67 variant amino acids (n = 8) shown in bold.) | |
| huJ591scFv | EVQLVQSGAEVKKPGASVKISCKTSGYTFTEYTIHWVKQASGKGLEW | 77 |
| | IGNINPNNGGTTYNQKFEDRATLTVDKSTSTAYMELSSLRSEDTAVYY | |
| | CAAGWNFDYWGQGTTVTVSSGSTSGGGSGGGSGGGGSSDIVMTQ | |
| | SPSSLSASVGDRVTITCKASQDVGTAVDWYQQKPGKAPKLLIYWAST | |
| | RHTGVPDRFTGSGSGTDFTLTISSLQPEDFADYFCQQYNSYPLTFGG | |
| | GTKLEIK | |
| N-Terminus of | EPDHLQRVSLPRMVYPQPKVLTPCRKDVLVVTPWLAPIVWEGTFNIDI | 78 |
| Human GTA (aa | LNEQFRLQNTTIGLTVFAIKKYVAFLKLFLETAEKHFMVGHRVHYYVFT | |
| 57-354) | DQPAAVPRVTLGTGRQLSVLEVRAYKRWQDVSMRRMEMISDFCERR | |
| | FLSEVDYLVCVDVDMEFRDHVGVEILTPLFGTLHPGFYGSSREAFTYE | |
| | RRPQSQAYIPKDEGDFYYLGGFFGGSVQEVQRLTRACHQAMMVDQ | |
| | ANGIEAVWHDESHLNKYLLRHKPTKVLSPEYLWDQQLLGWPAVLRKL | |
| | RFTAVPKNHQAVRNP | |
| Trastuzumab | EVQLVESGGGLVQPGGSLRLSCAASGFNIKDTYIHWVRQAPGKGLE | 79 |
| (4D5) Heavy Chain | WVARIYPTNGYTRYADSVKGRFTISADTSKNTAYLQMNSLRAEDTAV | |
| | YYCSRWGGDGFYAMDYWGQGTLVTVSSASTKGPSVFPLAPSSKST | |
| | SGGTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYKCK | |
| | VSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRW | |
| | QQGNVFSCSVMHEALHNHYTQKSLSLSPG | |
| Trastuzumab | DIQMTQSPSSLSASVGDRVTITCRASQDVNTAVAWYQQKPGKAPKLL | 80 |
| (4D5) Light Chain | IYSASFLYSGVPSRFSGSRSGTDFTLTISSLQPEDFATYYCQQHYTTP | |
| | PTFGQGTKVEIKRTVAAPSVFIFPPSDEQLKSGTASVVCLLNNFYPRE | |
| | AKVQWKVDNALQSGNSQESVTEQDSKDSTYSLSSTLTLSKADYEKH | |
| | KVYACEVTHQGLSSPVTKSFNRGEC | |
| Trastuzumab | EVQLVESGGGLVQPGGSLRLSCAASGFNIKDTYIHWVRQAPGKGLE | 81 |
| (4D5) scFv | WVARIYPTNGYTRYADSVKGRFTISADTSKNTAYLQMNSLRAEDTAV | |
| | YYCSRWGGDGFYAMDYWGQGTLVTVSSGGGGSGGGGSGGGGSDI | |
| | QMTQSPSSLSASVGDRVTITCRASQDVNTAVAWYQQKPGKAPKLLIY | |
| | SASFLYSGVPSRFSGSRSGTDFTLTISSLQPEDFATYYCQQHYTTPPT | |
| | FGQGTKVEIK | |

huJ591-GTB

huJ591-GTB (H chain) (SEQ ID NO: 34) was constructed by ligating huJ591 heavy chain (SEQ ID NO: 68) to the N-terminus of human GTB (aa 57-354) (SEQ ID NO: 69). huJ591-LC (L chain) (SEQ ID NO: 36) was constructed by adding 6His-tag (SEQ ID NO: 70) to the C-terminus of huJ591-LC (SEQ ID NO: 70) to facilitate affinity chromatography purification.

DNA sequence encoding H and L chain were subcloned into a pcDNA 3.1 expression vector. Protein production was done using transient expression method by co-transfection of H and L chain into CHO cells. huJ591-GTB fusion protein was purified from the cell culture supernatant by Nickel affinity chromatography and evaluated by SDS-PAGE.

huJ591Fab-GTB

huJ591Fab-GTB (H chain) (SEQ ID NO: 37) was constructed by ligating a truncated fragment of huJ591 heavy chain (VH-CH1-partial hinge sequence) (SEQ ID NO:72) to the N-terminus of human GTB (aa 57-354) (SEQ ID NO: 69). A Myc/his tag (SEQ ID NO: 73) was added to the C-terminus to facilitate monitoring expression and affinity chromatography purification. huJ591-LC (L chain) (SEQ ID NO: 39) encodes huJ591 light chain sequence (SEQ ID NO: 71).

DNA sequence encoding H and L chain were subcloned into pcDNA 3.1 expression vector. Protein production was carried out using the transient expression method by co-transfection of H-chain and L-chain into CHO cells. Fab-GTB fusion protein was purified from the cell culture supernatant by Nickel affinity chromatography and evaluated by SDS-PAGE.

huJ591-HC67-GTB

huJ591-HC67-GTB (H chain) (SEQ ID NO: 40) was constructed by ligating huJ591 heavy chain (SEQ ID NO: 74) to the N-terminus of human GTB (aa 57-354) (SEQ ID No: 69) via a (G₄S)₃ (SEQ ID NO: 67) linker. Changed aa are labeled in bold double underline in Table 4 and bold text in Table 7. J591-LC (L chain) (SEQ ID NO: 42) was constructed by adding 6His-tag (SEQ ID NO: 70) to the C-terminus of the J591-LC (SEQ ID NO: 71) to facilitate affinity chromatography purification.

Monomeric Fc fusion protein production was carried out as follows. DNA sequence encoding J591HC67-GTB and L chain were synthesized, subcloned into an expression vector, and co-transfected into CHO cells. Cell culture supernatant was harvested. J591HC67-GTB fusion protein was purified using Nickel affinity chromatography and evaluated by SDS-PAGE.

huJ591-HC67-GTB54aa

huJ591-HC67-GTB54aa (H chain) (SEQ ID NO: 43) was modified from huJ591-HC67-GTB (H chain) (SEQ ID NO: 74) by adding a 54aa tail (SEQ ID NO: 75) at the C-terminus of GTB. huJ591-LC (L chain) (SEQ ID NO: 45) was constructed by adding 6His-tag (SEQ ID NO: 70) to the C-terminus to facilitate affinity chromatography purification.

Protein production was carried out using the transient expression method by co-transfection of H chain and L chain into CHO cells. huJ591-GTB fusion protein was purified from the cell culture supernatant by Nickel affinity chromatography and evaluated by SDS-PAGE.

huJ591scFv-Fc67-GTB

huJ591scFv-Fc67-GTB (SEQ ID NO: 46) encodes (from N to C terminus) huJ591 single chain variable fragment (scFv)/J591 Fc fragment (SEQ ID NO: 76), human GTB (aa 57-354) (SEQ ID NO: 69). A (G₄S)₃ (SEQ ID NO: 67) linker was added in between Fe (SEQ ID NO: 76) and GTB (SEQ ID NO: 69).

A DNA sequence encoding huJ591scFv-Fc67-GTB (SEQ ID NO: 46) was synthesized, subcloned into an expression vector, and transfected into CHO cells. Cell culture supernatant was harvested. huJ591scFv-Fc67-GTB (SEQ ID NO: 46) fusion protein was purified using Nickel affinity chromatography and evaluated by SDS-PAGE.

huJ591scFv-GTB

huJ591scFv-GTB (SEQ ID NO: 48) encodes (from N to C terminus) the de-immunized version of huJ591 single chain variable fragment (scFv) (SEQ ID NO: 77), human GTB (aa 57-354) (SEQ ID NO: 69). A (G₄S)₃ (SEQ ID NO: 67) linker was added in between scFv (SEQ ID NO: 77) and GTB (SEQ ID NO: 69). A Myc/His tag (SEQ ID NO: 73) was added to the C-terminus to facilitate monitoring expression and affinity chromatography purification.

A DNA sequence encoding huJ591scFv-GTB (SEQ ID NO: 48) was subcloned into a pcDNA 3.1 expression vector. Protein production was carried out using the transient expression method by transfection of the plasmid into CHO cells. huJ591scFv-GTB fusion protein was purified from the cell culture supernatant by Nickel affinity chromatography and evaluated by SDS-PAGE.

### GTA Constructs

The human GTA (aa 57-354) sequence (SEQ ID NO: 78) was also used in place of the GTB sequence (SEQ ID NO: 69) in the DNA constructs described above in order to generate the following recombinant proteins: huJ591-GTA (SEQ ID NO: 35), huJ591Fab-GTA (SEQ ID NO: 38), huJ591-HC67-GTA (SEQ ID NO: 41), huJ591scFv-Fc67-GTA (SEQ ID NO: 47), and huJ591scFv-GTA (SEQ ID NO: 49).

### Trastuzumab (4D5) Constructs

To target HER2 on breast and other cancers, the Trastuzumab (4D5) sequence was used in place of the huJ591 sequence in the DNA constructs described herein to generate the following recombinant proteins: 4D5-GTA (SEQ ID NO: 51), 4D5Fab-GTA (SEQ ID NO: 54), 4D5HC67-GTA (SEQ ID NO: 57), 4D5scFv-Fc67-GTA (SEQ ID NO: 60), 4D5scFv-GTA (SEQ ID NO: 62), 4D5-GTB (SEQ ID NO: 50), 4D5Fab-GTB (SEQ ID NO: 53), 4D5HC67-GTB (SEQ ID NO: 56), 4D5scFv-Fc67-GTB (SEQ ID NO: 59), and 4D5scFv-GTB (SEQ ID NO: 61).

Although preferred embodiments have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims which follow.

What is claimed:

1. A bi-functional therapeutic for treating cancer comprising:
a targeting component which targets a tumor-associated antigen and
an enzyme which, when delivered to a tumor by said targeting component, enzymatically converts the tumor phenotype to that of an incompatible allograft or xenograft, said enzyme being coupled to said targeting component.

2. The bi-functional therapeutic according to claim 1, wherein the tumor-associated antigen is selected from the group consisting of FOLH1/PSMA, VEGFR, CD19, CD20, CD25, CD30, CD33, CD38, CD52, B cell Maturation Antigen (BCMA), CD79, Somatostatin receptor, 5T4, gp100, CEA, melan A/MART-1, MAGE, NY-ESO-1, PSA, tyrosinase, HER-2, HER-3, EGFR, hTERT, MUC1, mesothelin, Nectin-4, TROP-2, Tissue Factor, and CA-125.

3. The bi-functional therapeutic according to claim 1, wherein the targeting component is selected from the group consisting of an antibody or antigen-binding fragment thereof, a protein, a peptide, an aptamer and a small molecule ligand.

4. The bi-functional therapeutic according to claim 3, wherein the targeting component is a peptide linked to the enzyme via a peptide bond.

5. The bi-functional therapeutic according to claim 4, wherein the targeting component is an antibody or antigen-binding derivative or fragment thereof.

6. The bi-functional therapeutic according to claim 1, wherein the targeting component and the enzyme are genetically engineered to produce a fusion protein.

7. The bi-functional therapeutic according to claim 1, wherein the targeting component and the enzyme are chemically linked.

8. The bi-functional therapeutic according to claim 3, wherein the targeting component is a small molecule ligand chemically linked to the enzyme with an intervening polyethylene glycol (PEG) spacer.

9. The bi-functional therapeutic according to claim 8, wherein the targeting component is ACUPA [2-(3-((S)-5-Amino-1carboxpentyl)ureido)pentanedioic Acid] chemically linked to the enzyme with an intervening PEG spacer.

10. The bi-functional therapeutic according to claim 1, wherein the enzyme is an enzyme involved in post-translational modification and is selected from the group consisting of a transferase and glycosyltransferase.

11. The bi-functional therapeutic according to claim 10, wherein the enzyme involved in post-translational modification is a transferase.

12. The bi-functional therapeutic according to claim 11, wherein the transferase is a glycosyltransferase.

13. The bi-functional therapeutic according to claim 12, wherein the glycosyltransferase is selected from the group consisting of glycosyltransferase A (Alpha 1-3-N-Acetylgalactosaminyltransferase), glycosyltransferase B (alpha 1-3-galactosyltransferase), alpha-gal-transferase, glycosyltransferase A (Gly268Ala), and fucosyltransferase.

14. The bi-functional therapeutic according to claim 1, wherein the enzyme comprises an appended second amino acid sequence at its C-terminus.

15. The bi-functional therapeutic according to claim 14, wherein the second amino acid sequence includes a cleavable amino acid sequence between the enzyme and the appended second sequence.

16. The bi-functional therapeutic according to claim 15, wherein the cleavable amino acid sequence is cleavable by PSA, matrix metalloproteinases, or cathepsin B.

17. The bi-functional therapeutic according to claim 1, wherein the tumor having the tumor-associated antigen expresses an H-antigen.

18. The bi-functional therapeutic according to claim 1, wherein the tumor having the tumor-associated antigen is from a cancer selected from the group consisting of lung cancer, gastric cancer, colorectal cancer, breast cancer, prostate cancer, blood cancer, cervical cancer, endometrial cancer, ovarian cancer, bladder cancer, renal cancer, brain cancer, hepatic cancer, esophageal cancer, adrenal cancer, head and neck cancer, melanoma, and pancreatic cancer.

19. The bi-functional therapeutic according to claim 18, wherein the cancer is prostate cancer.

20. The bi-functional therapeutic according to claim 19, wherein the targeting component targets the prostate-specific membrane antigen (PSMA)/Folate hydrolase 1 (FOLH1) receptor.

21. The bi-functional therapeutic according to claim 19, wherein the targeting component is a PSMA receptor antibody or derivative of a PSMA receptor antibody.

22. The bi-functional therapeutic according to claim 19, wherein the targeting component is an antibody selected from the group consisting of J591, J415, J533, and E99.

23. The bi-functional therapeutic according to claim 17, wherein the cancer is breast cancer.

24. The bi-functional therapeutic according to claim 23, wherein the targeting component targets an HER receptor family member.

25. The bi-functional therapeutic according to claim 24, wherein the targeting component is monoclonal antibody 4D5.

26. The bi-functional therapeutic according to claim 17, wherein the cancer is a blood cancer of B-cell lineage.

27. The bi-functional therapeutic according to claim 26, wherein the targeting component targets CD19.

28. The bi-functional therapeutic according to claim 27, wherein the targeting component is the monoclonal antibody obexelimab or denintuzumab.

29. A method of treating cancer, said method comprising:
selecting a subject having cancer;
providing a bi-functional therapeutic according to any of claims 1-28; and
administering, to the selected subject, the bi-functional therapeutic under conditions effective to treat the cancer.

30. The method according to claim 29, wherein the subject is a human.

31. The method according to claim 29, wherein the tumor associated antigen is selected from the group consisting of FOLH1/PSMA, VEGFR, CD19, CD20, CD25, CD30, CD33, CD38, CD52, B Cell Maturation Antigen (BCMA), CD79, Somatostatin receptor, 5T4, gp100, CEA, melan A/MART-1, MAGE, NY-ESO-1, PSA, tyrosinase, HER-2, HER-3, EGFR, hTERT, MUC1, mesothelin, Nectin-4, TROP-2, Tissue Factor, and CA-125.

32. The method according to claim 29, wherein the targeting component is selected from the group consisting of an antibody or binding fragment thereof, a protein, a peptide, and a small molecule.

33. The method according to claim 32, wherein the targeting component is a peptide linked to the enzyme via a peptide bond.

34. The method according to claim 32, wherein the targeting component is an antibody or antigen-binding derivative or fragment thereof.

35. The method according to claim 29, wherein the targeting component and the enzyme are genetically engineered to produce a fusion protein.

36. The method according to claim 29, wherein the targeting component and the enzyme are chemically linked.

37. The method according to claim 29, wherein the targeting component is a small molecule/ligand chemically linked to the enzyme with an intervening polyethylene glycol (PEG) spacer.

38. The method according to claim 37, wherein the targeting component is ACUPA [2-(3-((S)-5-Amino-1-carbopentyl)ureido)pentanedioic Acid) chemically linked to the enzyme with an intervening PEG spacer.

39. The method according to claim 29, wherein the enzyme is an enzyme involved in post-translational modification and is selected from the group consisting of glycosylation, a transferase, and glycosyltransferase.

40. The method according to claim 39, wherein the enzyme involved in post-translational modification is a transferase.

41. The method according to claim 40, wherein the transferase is a glycosyltransferase.

42. The method according to claim 41, wherein the glycosyltransferase is selected from the group consisting of glycosyltransferase A, glycosyltransferase B, alpha-gal-transferase, glycosyltransferase A (Gly268Ala), and fucosyltransferase.

43. The method according to claim 29, wherein the enzyme comprises an appended second amino acid sequence at its C-terminus.

44. The method according to claim 43, wherein the second amino acid sequence comprises a cleavable amino acid sequence between the enzyme and the appended second sequence.

45. The method according to claim 44, wherein the cleavable amino acid sequence is cleavable by PSA, matrix metalloproteinases, or cathepsin B.

46. The method according to claim 29, wherein the cancer expresses an H-antigen.

47. The method according to claim 29, wherein the cancer is selected from the group consisting of lung cancer, gastric cancer, colorectal cancer, breast cancer, prostate cancer, blood cancer, cervical cancer, endometrial cancer, ovarian cancer, bladder cancer, renal cancer, brain cancer, hepatic cancer, esophageal cancer, adrenal cancer, head and neck cancer, melanoma, and pancreatic cancer.

48. The method according to claim 47, wherein the cancer is prostate cancer.

49. The method according to claim 48, wherein the targeting component targets the prostate-specific membrane antigen (PSMA)/Folate hydrolase 1 (FOLH1) receptor.

50. The method according to claim 49, wherein the targeting component is a PSMA receptor antibody or derivative of the PSMA receptor antibody.

51. The method according to claim 50, wherein the targeting component is an antibody selected from the group consisting of J591, J415, J533, and E99.

52. The method according to claim 47, wherein the cancer is breast cancer.

53. The method according to claim 52, wherein the targeting component targets an HER receptor family member.

54. The method according to claim 53, wherein the targeting component is monoclonal antibody 4D5.

55. The method according to claim 47, wherein the cancer is a blood cancer of B-cell lineage.

56. The method according to claim 55, wherein the targeting component targets CD19.

57. The method according to claim 56, wherein the targeting component is monoclonal antibody obexelimab or denintuzumab.

58. The method according to claim 29, wherein said administering further comprises:
administering uridine diphosphate-galactose (UDP-gal), uridine diphosphate-N-acetylgalactosamine (UDP-NAcGal), and/or guanosine diphosphate-fucose (GDP-fucose).

59. The method according to claim 49, wherein the targeting component targets PSMA receptor on tumor vascular endothelium.

60. A pharmaceutical composition comprising:
the bi-functional therapeutic according to any of claims 1-28 and
a pharmaceutically acceptable carrier.

61. A nucleic acid molecule encoding the bi-functional therapeutic according to any of claims 1-28.

62. A nucleic acid construct comprising the nucleic acid molecule according to claim 61.

63. A recombinant expression vector comprising the nucleic acid molecule according to claim 61.

64. A recombinant host cell transformed with the nucleic acid molecule according to claim 61.

65. A bi-functional therapeutic for treating cancer comprising:
a targeting component which targets the prostate-specific membrane antigen (PSMA)/Folate hydrolase 1 (FOLH1) receptor and
a glycosyltransferase which, when delivered to a tumor by said targeting component, enzymatically converts the tumor phenotype to that of an incompatible allograft or xenograft, said glycosyltransferase being coupled to said targeting component.

66. The bi-functional therapeutic of claim 65, wherein said targeting component comprises a heavy chain variable region, wherein said heavy chain variable region comprises:
a complementarity-determining region 1 (CDR-H1) comprising an amino acid sequence of SEQ ID NO: 10, or a modified amino acid sequence of SEQ ID NO: 10, said modified sequence having at least 80% sequence identity to SEQ ID NO: 10;
a complementarity-determining region 2 (CDR-H2) comprising an amino acid sequence of SEQ ID NO: 13, or a modified amino acid sequence of SEQ ID NO: 13, said modified sequence having at least 80% sequence identity to SEQ ID NO: 13; and
a complementarity-determining region 3 (CDR-H3) comprising an amino acid sequence of SEQ ID NO: 16, or a modified amino acid sequence of SEQ ID NO: 16, said modified sequence having at least 80% sequence identity to SEQ ID NO: 16.

67. The bi-functional therapeutic of claim 65, wherein said targeting component comprises a heavy chain variable region comprising an amino acid sequence that is at least 80% identical to SEQ ID NO: 28.

68. The bi-functional therapeutic of claim 66 or 67, wherein said targeting component further comprises a light chain variable region, wherein said light chain variable region comprises:
a complementarity-determining region 1 (CDR-L1) having an amino acid sequence of SEQ ID NO: 19, or a modified amino acid sequence of SEQ ID NO: 19, said modified sequence having at least 80% sequence identity to SEQ ID NO: 19;
a complementarity-determining region 2 (CDR-L2) having an amino acid sequence of SEQ ID NO: 22, or a modified amino acid sequence of SEQ ID NO: 22, said modified sequence having at least 80% sequence identity to SEQ ID NO: 22; and
a complementarity-determining region 3 (CDR-L3) having an amino acid sequence of SEQ ID NO: 25, or a modified amino acid sequence of SEQ ID NO: 25, said modified sequence having at least 80% sequence identity to SEQ ID NO: 25.

69. The bi-functional therapeutic of claim 65, wherein said targeting component comprises a light chain variable region comprising an amino acid sequence that is at least 80% identical to SEQ ID NO: 29.

70. The bi-functional therapeutic of claim 65, wherein said targeting component comprises a heavy chain variable region comprising the CDR-H1 of SEQ ID NO: 10, the CDR-H2 of SEQ ID NO: 13, and the CDR-H3 of SEQ ID NO: 16, and a light chain variable region comprising the CDR-L1 of SEQ ID NO: 19, the CDR-L2 of SEQ ID NO: 22, and the CDR-L3 of SEQ ID NO: 25.

71. The bi-functional therapeutic of claim 65, wherein said targeting component comprises a heavy chain variable region comprising an amino acid sequence that is at least 80% identical to SEQ ID NO: 28 and a light chain variable region comprising an amino acid sequence that is at least 80% identical to SEQ ID NO: 29.

72. The bi-functional therapeutic of claim 65, wherein said targeting component further comprises a signaling peptide, optionally wherein the signaling peptide has the sequence of amino acids 1-19 of SEQ ID NO: 34.

73. The bi-functional therapeutic of claim 65, wherein the glycosyltransferase is selected from the group consisting of glycosyltransferase A (Alpha 1-3-N-Acetylgalactosaminyltransferase) and glycosyltransferase B (alpha 1-3-galactosyltransferase).

74. The bi-functional therapeutic of claim 65, wherein the bi-functional therapeutic comprises:
(i) a first protein comprising the amino acid sequence of SEQ ID NO: 34 or SEQ ID NO: 35 and a second protein comprising the amino acid sequence of SEQ ID NO: 36;
(ii) a first protein comprising the amino acid sequence of SEQ ID NO: 37 or SEQ ID NO: 38 and a second protein comprising the amino acid sequence of SEQ ID NO: 39;
(iii) a first protein comprising the amino acid sequence of SEQ ID NO: 40 or SEQ ID NO: 41 and a second protein comprising the amino acid sequence of SEQ ID NO: 42;
(iv) a first protein comprising the amino acid sequence of SEQ ID NO: 43 or SEQ ID NO: 44 and a second protein comprising the amino acid sequence of SEQ ID NO: 45;
(v) the amino acid sequence of SEQ ID NO: 46;
(vi) the amino acid sequence of SEQ ID NO: 47;
(vii) the amino acid sequence of SEQ ID NO: 48; or
(viii) the amino acid sequence of SEQ ID NO: 49.

75. A bi-functional therapeutic for treating cancer comprising:
a targeting component which targets a human epidermal growth factor receptor (HER) family member and
a glycosyltransferase which, when delivered to a tumor by said targeting component, enzymatically converts the tumor phenotype to that of an incompatible allograft or xenograft, said glycosyltransferase being coupled to said targeting component.

76. The bi-functional therapeutic of claim 75, wherein said targeting component comprises a heavy chain variable region, wherein said heavy chain variable region comprises:
a complementarity-determining region 1 (CDR-H1) comprising an amino acid sequence of SEQ ID NO: 11, or a modified amino acid sequence of SEQ ID NO: 11, said modified sequence having at least 80% sequence identity to SEQ ID NO: 11;
a complementarity-determining region 2 (CDR-H2) comprising an amino acid sequence of SEQ ID NO: 14, or a modified amino acid sequence of SEQ ID NO: 14, said modified sequence having at least 80% sequence identity to SEQ ID NO: 14; and
a complementarity-determining region 3 (CDR-H3) comprising an amino acid sequence of SEQ ID NO: 17, or a modified amino acid sequence of SEQ ID NO: 17, said modified sequence having at least 80% sequence identity to SEQ ID NO: 17.

77. The bi-functional therapeutic of claim 75, wherein said targeting component comprises a heavy chain variable region comprising an amino acid sequence that is at least 80% identical to SEQ ID NO: 30.

78. The bi-functional therapeutic of claim 76 or claim 77, wherein said targeting component further comprises a light chain variable region, wherein said light chain variable region comprises:
a complementarity-determining region 1 (CDR-L1) having an amino acid sequence of SEQ ID NO: 20, or a modified amino acid sequence of SEQ ID NO: 20, said modified sequence having at least 80% sequence identity to SEQ ID NO: 20;
a complementarity-determining region 2 (CDR-L2) having an amino acid sequence of SEQ ID NO: 23, or a modified amino acid sequence of SEQ ID NO: 23, said modified sequence having at least 80% sequence identity to SEQ ID NO: 23; and
a complementarity-determining region 3 (CDR-L3) having an amino acid sequence of SEQ ID NO: 26, or a modified amino acid sequence of SEQ ID NO: 26, said modified sequence having at least 80% sequence identity to SEQ ID NO: 26.

79. The bi-functional therapeutic of claim 75, wherein said targeting component comprises a light chain variable region comprising an amino acid sequence that is at least 80% identical to SEQ ID NO: 31.

80. The bi-functional therapeutic of claim 75, wherein said targeting component comprises a heavy chain variable region comprising the CDR-H1 of SEQ ID NO: 11, the CDR-H2 of SEQ ID NO: 14, and the CDR-H3 of SEQ ID NO: 17, and a light chain variable region comprising the CDR-L1 of SEQ ID NO: 20, the CDR-L2 of SEQ ID NO: 23, and the CDR-L3 of SEQ ID NO: 26.

81. The bi-functional therapeutic of claim 75, wherein said targeting component comprises a heavy chain variable region comprising an amino acid sequence that is at least 80% identical to SEQ ID NO: 30 and a light chain variable region comprising an amino acid sequence that is at least 80% identical to SEQ ID NO: 31.

82. The bi-functional therapeutic of claim 75, wherein said targeting component further comprises a signaling peptide, optionally wherein the signaling peptide has the sequence of amino acids 1-19 of SEQ ID NO: 50.

83. The bi-functional therapeutic of claim 75, wherein the glycosyltransferase is selected from the group consisting of glycosyltransferase A (Alpha 1-3-N-Acetylgalactosaminyltransferase) and glycosyltransferase B (alpha 1-3-galactosyltransferase).

84. The bi-functional therapeutic of claim 75, wherein the bi-functional therapeutic comprises:
(i) a first protein comprising the amino acid sequence of SEQ ID NO: 50 or SEQ ID NO: 51 and a second protein comprising the amino acid sequence of SEQ ID NO: 52;
(ii) a first protein comprising the amino acid sequence of SEQ ID NO: 53 or SEQ ID NO: 54 and a second protein comprising the amino acid sequence of SEQ ID NO: 55;
(iii) a first protein comprising the amino acid sequence of SEQ ID NO: 56 or SEQ ID NO: 57 and a second protein comprising the amino acid sequence of SEQ ID NO: 58;
(iv) the amino acid sequence of SEQ ID NO: 59;
(v) the amino acid sequence of SEQ ID NO: 60;
(vi) the amino acid sequence of SEQ ID NO: 61; or
(vii) the amino acid sequence of SEQ ID NO: 62.

85. A bi-functional therapeutic for treating cancer comprising:
a targeting component which targets CD19 and
a glycosyltransferase which, when delivered to a tumor by said targeting component, enzymatically converts the tumor phenotype to that of an incompatible allograft or xenograft, said glycosyltransferase being coupled to said targeting component.

86. The bi-functional therapeutic of claim 85, wherein said targeting component comprises a heavy chain variable region, wherein said heavy chain variable region comprises:
a complementarity-determining region 1 (CDR-H1) comprising an amino acid sequence of SEQ ID NO: 12, or a modified amino acid sequence of SEQ ID NO: 12, said modified sequence having at least 80% sequence identity to SEQ ID NO: 12;
a complementarity-determining region 2 (CDR-H2) comprising an amino acid sequence of SEQ ID NO: 15, or a modified amino acid sequence of SEQ ID NO: 15, said modified sequence having at least 80% sequence identity to SEQ ID NO: 15; and
a complementarity-determining region 3 (CDR-H3) comprising an amino acid sequence of SEQ ID NO: 18, or a modified amino acid sequence of SEQ ID NO: 18, said modified sequence having at least 80% sequence identity to SEQ ID NO: 18.

87. The bi-functional therapeutic of claim 85, wherein said targeting component comprises a heavy chain variable region comprising an amino acid sequence that is at least 80% identical to SEQ ID NO: 32.

88. The bi-functional therapeutic of claim 86 or claim 87, wherein said targeting component further comprises a light chain variable region, wherein said light chain variable region comprises:
a complementarity-determining region 1 (CDR-L1) having an amino acid sequence of SEQ ID NO: 21, or a modified amino acid sequence of SEQ ID NO: 21, said modified sequence having at least 80% sequence identity to SEQ ID NO: 21;
a complementarity-determining region 2 (CDR-L2) having an amino acid sequence of SEQ ID NO: 24, or a modified amino acid sequence of SEQ ID NO: 24, said modified sequence having at least 80% sequence identity to SEQ ID NO: 24; and
a complementarity-determining region 3 (CDR-L3) having an amino acid sequence of SEQ ID NO: 27, or a modified amino acid sequence of SEQ ID NO: 27, said modified sequence having at least 80% sequence identity to SEQ ID NO: 27.

89. The bi-functional therapeutic of claim 85, wherein said targeting component comprises a light chain variable region comprising an amino acid sequence that is at least 80% identical to SEQ ID NO: 32.

90. The bi-functional therapeutic of claim 85, wherein said targeting component comprises a heavy chain variable region comprising the CDR-H1 of SEQ ID NO: 12, the CDR-H2 of SEQ ID NO: 15, and the CDR-H3 of SEQ ID NO: 18, and a light chain variable region comprising the CDR-L1 of SEQ ID NO: 21, the CDR-L2 of SEQ ID NO: 24, and the CDR-L3 of SEQ ID NO: 27.

91. The bi-functional therapeutic of claim 85, wherein said targeting component comprises a heavy chain variable region comprising an amino acid sequence that is at least 80% identical to SEQ ID NO: 32 and a light chain variable region comprising an amino acid sequence that is at least 80% identical to SEQ ID NO: 33.

92. The bi-functional therapeutic of claim 85, wherein said targeting component further comprises a signaling peptide, optionally wherein the signaling peptide has the sequence of amino acids 1-19 of SEQ ID NO: 63.

93. The bi-functional of claim 85, wherein the glycosyltransferase is selected from the group consisting of glycosyltransferase A (Alpha 1-3-N-Acetylgalactosaminyltransferase), glycosyltransferase B (alpha 1-3-galactosyltransferase), and Marmoset α-1,3 galactosyltransferase.

94. The bi-functional therapeutic of claim 85, wherein the bi-functional therapeutic comprises the amino acid sequence of SEQ ID NO: 63.


SEQUENCE LISTING (81 sequences)

SEQ ID NO: 1 - protein, 5 aa, Artificial Sequence
  linker
GGGGS

SEQ ID NO: 2 - protein, 31 aa, Artificial Sequence
  linker
  MISC_FEATURE (16)..(20): Ser at position 16 and Gly at positions 17-20 are either all present or all absent
  MISC_FEATURE (21)..(25): Ser at position 21 and Gly at positions 22-25 are either all present or all absent
  MISC_FEATURE (26)..(30): Ser at position 26 and Gly at positions 27-30 are either all present or all absent
SGGGGSGGGGSGGGGSGGGGSGGGGSGGGGS

SEQ ID NO: 3 - protein, 115 aa, Homo sapiens
  ref <308>: DATABASE ACCESSION NUMBER: GenBank Accession No. CCA78124.1
  ref <309>: DATABASE ENTRY DATE: 2016-07-25
  ref <313>: RELEVANT RESIDUES IN SEQ ID NO: (1)..(115)
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 4 - protein, 108 aa, Homo sapiens
  ref <308>: DATABASE ACCESSION NUMBER: CCA78125.1
  ref <309>: DATABASE ENTRY DATE: 2016-07-25
  ref <313>: RELEVANT RESIDUES IN SEQ ID NO: (1)..(108)
DIVMTQSHKFMSTSVGDRVSIICKASQDVGTAVDWYQQKPGQSPKLLIYWASTRHTGVPDRFTGSGSGTDFTLAITNVQSEDLADYFCQQYNSYPLTFGAGTKLEIKR

SEQ ID NO: 5 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 354 aa, Homo sapiens
MAEVLRTLAGKPKCHALRPMILFLIMLVLVLFGYGVLSPRSLMPGSLERGFCMAVREPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 8 - protein, 229 aa, Homo sapiens
  ref <308>: DATABASE ACCESSION NUMBER: ABL75287.1
  ref <309>: DATABASE ENTRY DATE: 2016-07-14
  ref <313>: RELEVANT RESIDUES IN SEQ ID NO: (1)..(229)
YVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 9 - protein, 354 aa, Homo sapiens [sequence omitted]

SEQ ID NO: 10 - protein, 10 aa, Artificial Sequence
  J591 HCDR1
GYTFTEYTIH

SEQ ID NO: 11 - protein, 10 aa, Artificial Sequence
  4D5 HCDR1
GFNIKDTYIH

SEQ ID NO: 12 - protein, 5 aa, Artificial Sequence
  obexelimab HCDR1
SYVMH

SEQ ID NO: 13 - protein, 17 aa, Artificial Sequence
  J591 HCDR2
NINPNNGGTTYNQKFED

SEQ ID NO: 14 - protein, 17 aa, Artificial Sequence
  4D5 HCDR2
RIYPTNGYTRYADSVKG

SEQ ID NO: 15 - protein, 17 aa, Artificial Sequence
  obexelimab HCDR2
YINPYNDGTKYNEKFQG

SEQ ID NO: 16 - protein, 6 aa, Artificial Sequence
  J591 HCDR3
GWNFDY

SEQ ID NO: 17 - protein, 12 aa, Artificial Sequence
  4D5 HCDR3
WGGDGFYAMDYW

SEQ ID NO: 18 - protein, 12 aa, Artificial Sequence
  obexelimab HCDR3
GTYYYGTRVFDY

SEQ ID NO: 19 - protein, 11 aa, Artificial Sequence
  J591 LCDR1
KASQDVGTAVD

SEQ ID NO: 20 - protein, 12 aa, Artificial Sequence
  4D5 LCDR1
RASQDVNTAVAW

SEQ ID NO: 21 - protein, 16 aa, Artificial Sequence
  obexelimab LCDR1
RSSKSLQNVNGNTYLY

SEQ ID NO: 22 - protein, 7 aa, Artificial Sequence
  J591 LCDR2
WASTRHT

SEQ ID NO: 23 - protein, 7 aa, Artificial Sequence
  4D5 LCDR2
SASFLYS

SEQ ID NO: 24 - protein, 7 aa, Artificial Sequence
  obexelimab LCDR2
RMSNLNS

SEQ ID NO: 25 - protein, 9 aa, Artificial Sequence
  J591 LCDR3
QQYNSYPLT

SEQ ID NO: 26 - protein, 8 aa, Artificial Sequence
  4D5 LCDR3
QQHYTTPP

SEQ ID NO: 27 - protein, 9 aa, Artificial Sequence
  obexelimab LCDR3
MQHLEYPIT

SEQ ID NO: 28 - protein, 115 aa, Artificial Sequence
  J591 variable heavy chain
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 29 - protein, 107 aa, Artificial Sequence
  J591 variable light chain
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 30 - protein, 120 aa, Artificial Sequence [sequence omitted]

SEQ ID NO: 31 - protein, 109 aa, Artificial Sequence
  4D5 variable light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 32 - protein, 121 aa, Artificial Sequence
  obexelimab variable heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 33 - protein, 112 aa, Artificial Sequence
  obexelimab variable light chain
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 34 - protein, 777 aa, Artificial Sequence
  Signal peptide-huJ591 H chain-GTB
MGWSCIILFLVATATGVHSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 35 - protein, 777 aa, Artificial Sequence
  Signal peptide-huJ591 H chain-GTA
MGWSCIILFLVATATGVHSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 36 - protein, 239 aa, Artificial Sequence
  Signal peptide- huJ591-LC-his tag
MGWSCIILFLVATATGVHSDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 37 - protein, 564 aa, Artificial Sequence
  Signal peptide-huJ591Fab-GTB-Myc-his tag
MGWSCIILFLVATATGVHSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 38 - protein, 564 aa, Artificial Sequence
  Signal peptide-huJ591Fab-GTA-Myc-his tag
MGWSCIILFLVATATGVHSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 39 - protein, 233 aa, Artificial Sequence
  Signal peptide- huJ591-LC
MGWSCIILFLVATATGVHSDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 758 aa, Artificial Sequence
  huJ591-HC67-GTB
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 41 - protein, 758 aa, Artificial Sequence
  huJ591-HC67-GTA
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 42 - protein, 220 aa, Artificial Sequence
  huJ591-LC-his tag
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 43 - protein, 831 aa, Artificial Sequence
  huJ591 HC67 -GTB-54aa
MNFGLRLIFLVLTLKGVQCEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPEFEQKLISEEDLNSADIHHTGARSSAHLELTADYKDHDGDYKDHDIDYKDDDDK

SEQ ID NO: 44 - protein, 831 aa, Artificial Sequence
  huJ591-HC67-GTA-54aa
MNFGLRLIFLVLTLKGVQCEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPEFEQKLISEEDLNSADIHHTGARSSAHLELTADYKDHDGDYKDHDIDYKDDDDK

SEQ ID NO: 45 - protein, 220 aa, Artificial Sequence; identical to SEQ ID NO: 42
  huJ591-LC-his tag

SEQ ID NO: 46 - protein, 773 aa, Artificial Sequence
  huJ591scFv-Fc67-GTB-his tag
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPHHHHHH

SEQ ID NO: 47 - protein, 773 aa, Artificial Sequence
  h591scFv-Fc67-GTA-his tag
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPHHHHHH

SEQ ID NO: 48 - protein, 597 aa, Artificial Sequence
  huJ591scFv-GTB-Myc-His tag
METDTLLLWVLLLWVPGSTGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGGGSGGGSGGGGSSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 49 - protein, 597 aa, Artificial Sequence
  huJ591scFv-GTA-Myc-His tag
METDTLLLWVLLLWVPGSTGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGGGSGGGSGGGGSSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 50 - protein, 781 aa, Artificial Sequence
  Signal peptide-4D5 H chain-GTB
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 51 - protein, 781 aa, Artificial Sequence
  Signal peptide-4D5 H chain -GTA
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 52 - protein, 239 aa, Artificial Sequence
  Signal peptide- 4D5-LC -his tag
MGWSCIILFLVATATGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 53 - protein, 569 aa, Artificial Sequence
  Signal peptide-4D5Fab-GTB-Myc-his tag
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 54 - protein, 569 aa, Artificial Sequence
  Signal peptide-4D5Fab-GTA-Myc-his tag
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 55 - protein, 233 aa, Artificial Sequence
  Signal peptide- 4D5-LC
MGWSCIILFLVATATGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 541 aa, Artificial Sequence
  4D5HC67-GTB
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 57 - protein, 541 aa, Artificial Sequence
  4D5HC67-GTA
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 58 - protein, 220 aa, Artificial Sequence
  4D5-LC-his tag
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 59 - protein, 561 aa, Artificial Sequence
  4D5scFv-Fc67-GTB-his tag
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPHHHHHH

SEQ ID NO: 60 - protein, 561 aa, Artificial Sequence
  4D5scFv-Fc67-GTA-his tag
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPHHHHHH

SEQ ID NO: 61 - protein, 599 aa, Artificial Sequence
  4D5scFv-GTB-Myc-His tag
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 62 - protein, 599 aa, Artificial Sequence
  4D5scFv-GTA-Myc-His tag
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNPAAAEQKLISEEDLNGAVEHHHHHH

SEQ ID NO: 63 - protein, 584 aa, Artificial Sequence
  human IL2 signal peptide-Obexelimab-scFv- Marmoset alpha-1,3 galactosyltransferase (aa90-376)-his tag
MGWSCIILFLVATATGVHSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGGGSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSELRLWDWFNPKKRPEVMTVTQWKAPVVWEGTYNKAILENYYAKQKITVGLTVFAIGRYIEHYLEEFVTSANRYFMVGHKVIFYVMVDDVSKAPFIELGPLRSFKVFEVKPEKRWQDISMMRMKTIGEHILAHIQHEVDFLFCMDVDQVFQDHFGVETLGQSVAQLQAWWYKADPDDFTYERRKESAAYIPFGQGDFYYHAAIFGGTPIQVLNITQECFKGILLDKKNDIEAEWHDESHLNKYFLLNKPSKILSPEYCWDYHIGLPSDIKTVKLSWQTKEYNLVRKNVGGGSHHHHHH

SEQ ID NO: 64 - protein, 298 aa, Homo sapiens
EPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 65 - protein, 297 aa, Homo sapiens
EPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRN

SEQ ID NO: 66 - protein, 291 aa, Unknown
  Protein
ELRLWDWFNPKKRPEVMTVTQWKAPVVWEGTYNKAILENYYAKQKITVGLTVFAIGRYIEHYLEEFVTSANRYFMVGHKVIFYVMVDDVSKAPFIELGPLRSFKVFEVKPEKRWQDISMMRMKTIGEHILAHIQHEVDFLFCMDVDQVFQDHFGVETLGQSVAQLQAWWYKADPDDFTYERRKESAAYIPFGQGDFYYHAAIFGGTPIQVLNITQECFKGILLDKKNDIEAEWHDESHLNKYFLLNKPSKILSPEYCWDYHIGLPSDIKTVKLSWQTKEYNLVRKNVGGGS

SEQ ID NO: 67 - protein, 15 aa, Artificial Sequence
  linker
GGGGSGGGGSGGGGS

SEQ ID NO: 68 - protein, 445 aa, Artificial Sequence
  huJ591 heavy chain
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 298 aa, Homo sapiens
EPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVGAYKRWQDVSMRRMEMISDFCERRFLSEVDYLVCVDVDMEFRDHVGVEILTPLFGTLHPSFYGSSREAFTYERRPQSQAYIPKDEGDFYYMGAFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 70 - protein, 6 aa, Artificial Sequence
  6 his tag
HHHHHH

SEQ ID NO: 71 - protein, 214 aa, Artificial Sequence
  huJ591-LC
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 223 aa, Artificial Sequence
  huJ591 heavy chain
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 73 - protein, 24 aa, Artificial Sequence
  Myc-his tag
AAAEQKLISEEDLNGAVEHHHHHH

SEQ ID NO: 74 - protein, 445 aa, Artificial Sequence
  huJ591 heavy chain
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 75 - protein, 54 aa, Artificial Sequence
  54aa tail
EFEQKLISEEDLNSADIHHTGARSSAHLELTADYKDHDGDYKDHDIDYKDDDDK

SEQ ID NO: 76 - protein, 454 aa, Artificial Sequence
  huJ591scFv/Fc
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 77 - protein, 240 aa, Artificial Sequence
  huJ591scFv
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGGGSGGGSGGGGSSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 78 - protein, 298 aa, Homo sapiens; identical to SEQ ID NO: 64

SEQ ID NO: 79 - protein, 449 aa, Artificial Sequence
  Trastuzumab (4D5) Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 214 aa, Artificial Sequence; identical to SEQ ID NO: 6
  Trastuzumab (4D5) Light Chain

SEQ ID NO: 81 - protein, 242 aa, Artificial Sequence
  Trastuzumab (4D5) scFv
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK